(19) United States
(12) Patent Application Publication
(10) Pub. No.: US 2024/0374749 A1
(43) Pub. Date: Nov. 14, 2024

(54) ANTIBODY-EXATECAN CONJUGATES

(71) Applicant: Glykos Finland Oy, Helsinki (FI)

(72) Inventors: Tero SATOMAA, Helsinki (FI); Juhani SAARINEN, Helsinki (FI)

(73) Assignee: Glykos Finland Oy, Helsinki (FI)

(21) Appl. No.: 18/602,451

(22) Filed: Mar. 12, 2024

(30) Foreign Application Priority Data
Mar. 13, 2023 (FI) 20235292

(51) Int. Cl. A61K 47/68; A61P 35/00

(52) U.S. Cl. CPC A61K 47/6889; A61K 47/68037; A61K 47/6849; A61K 47/6855; A61K 47/6865; A61K 47/6867; A61P 35/00

(57) ABSTRACT

Linker-payload conjugates and targeting unit-linker-payload conjugates are disclosed.

### RELATED APPLICATIONS

This application claims priority to FI 20235292 filed on Mar. 13, 2023 which is incorporated herein into this application in its entirety.

### TECHNICAL FIELD

The present disclosure relates to a linker-payload conjugate, a targeting unit-linker-payload conjugate, methods for preparing the same, a pharmaceutical composition and a method of treating and/or modulating the growth of and/or prophylaxis of tumor cells.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A linker-payload conjugate is disclosed. The linker-payload conjugate may be represented by Formula I or Formula IB

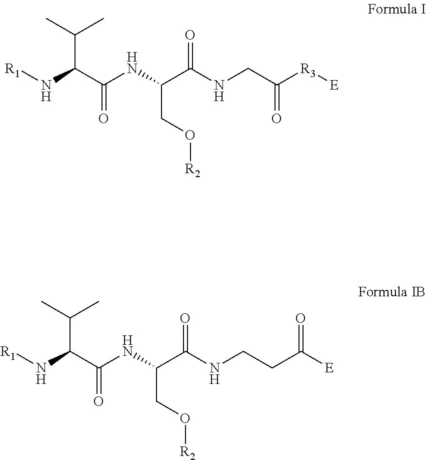

wherein R₁ is a spacer group selected from the group consisting of a maleimidoacetyl, maleimidoacetyl-β-alanyl, and a C₂-C₈ acyl group comprising a bioorthogonal conjugation group; R₂ is selected from the group consisting of a saccharide, a phosphate ester, a sulfate ester, a phosphodiester and a phosphonate; R₃ is a spacer group or absent, wherein the spacer group is selected from the group consisting of a prodrug group and —NH—CH₂—O—CH₂—C(O)—; and E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group.

A targeting unit-linker-payload conjugate is also disclosed. The targeting unit-linker-payload conjugate may be represented by Formula III or Formula IIIB

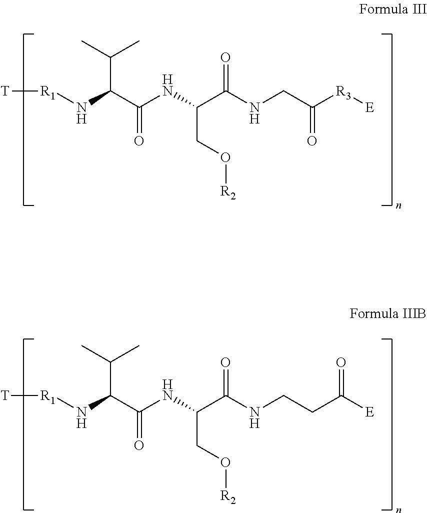

wherein T is a targeting unit; R₁ is a spacer group selected from the group consisting of

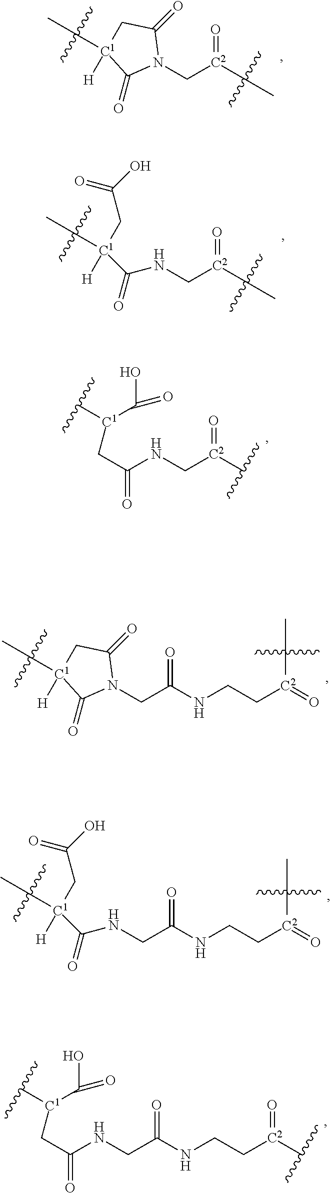

and a C₂-C₈ acyl group comprising a radical of a bioorthogonal conjugation group, wherein C¹ is covalently attached to the targeting unit, optionally covalently attached to to a sulphur of the targeting unit, and C² is covalently attached to the nitrogen of NH; R₂ is selected from the group consisting of a saccharide, a phosphate ester, a sulfate ester, a phosphodiester and a phosphonate; R₃ is a spacer group or absent, wherein the spacer group is selected from the group consisting of a prodrug group and —NH—CH₂—O—CH₂—C(O)—; E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group; and n≥1.

### FIGURE LEGENDS

FIG. 1A shows MALDI-TOF mass spectra of reduced flanvotumab antibody. y-axis shows relative signal intensity and x-axis shows m/z. L0=light chain without linker-payload, H0=heavy chain without linker-payload, 2+ shows that the heavy chain fragments have a charge of 2.

FIG. 1B shows MALDI-TOF mass spectra of flanvotumab-exatecan ADC FLExM01 with drug-to-antibody ratio DAR≈8 and non-stabilized maleimides. y-axis shows relative signal intensity and x-axis shows m/z. L0=light chain without linker-payload, L1=light chain with one linker-payload, H3=heavy chain with three linker-payload, and 2+ shows that the heavy chain fragments have a charge of 2.

FIG. 1C shows MALDI-TOF mass spectra of lintuzumab-exatecan ADC LNExM02 with DAR≈8 and non-stabilized maleimides. y-axis shows relative signal intensity and x-axis shows m/z. L0=light chain without linker-payload, H3=heavy chain with three linker-payload, and 2+ shows that the heavy chain fragments have a charge of 2.

FIG. 1D shows MALDI-TOF mass spectra of lintuzumab-exatecan ADC LNExM02 with DAR≈8 and stabilized maleimides. y-axis shows relative signal intensity and x-axis shows m/z. L0=light chain without linker-payload, L1=light chain with one linker-payload, H3=heavy chain with three linker-payload, and 2+ shows that the heavy chain fragments have a charge of 2.

FIG. 2A shows size-exclusion chromatography (SEC-HPLC) of flanvotumab-exatecan ADC FLExM01, showing very low aggregation with high-molecular weight aggregate level of 1.6%.

FIG. 2B shows reversed-phase chromatography (RP-HPLC) of reduced flanvotumab (lower panel) and flanvotumab-exatecan ADC FLExM01 (upper panel), showing homogeneous conjugation to L1 and H3 fragments and DAR=8.

FIG. 2C shows hydrophobic interaction chromatography (HIC-HPLC) of flanvotumab, flanvotumab-exatecan ADC FLExM01 and flanvotumab-deruxtecan ADC FLDxM01, showing that FLExM01 was more hydrophilic than FLDxM01, eluting closer to the naked antibody at 6.5 ml compared to 7.8 ml, respectively.

FIG. 3A shows in vitro cytotoxicity assays of exatecan ADCs, HER2+SK-BR-3 breast cancer cells were incubated with trastuzumab-exatecan ADC TRExM01, trastuzumab-deruxtecan ADC TRDxM01. IC50 values are shown in the figure and they indicate that TRExM01 (IC50-0.37 nM) was more cytotoxic to the HER2+cells than TRDxM01 (IC50=0.40 nM).

FIG. 3B shows in vitro cytotoxicity assays of exatecan ADCs, CD33+ MOLM-13 leukemia cells were incubated with lintuzumab-exatecan ADC LNExM01 and lintuzumab-deruxtecan ADC TRDxM01. IC50 values are shown in the figure and they indicate that LNExM01 was more cytotoxic to the CD33+ leukemia cells than LNDxM01.

FIG. 4A shows average tumor sizes in xenograft models. SK-MEL-30 melanoma cells were inoculated subcutaneously (s.c.) to athymic mice. Tumor mice were divided into groups with similar average tumor sizes when they reached about 200 mm³. 5 mice were treated with a single intravenous (i.v.) 10 mg/kg dose of flanvotumab antibody, 5 mice were treated with a single i.v. 10 mg/kg dose of flanvotumab-exatecan ADC FLExM, and 10 mice were left untreated. Tumor growth was inhibited the most in ADC-treated mice.

FIG. 4B shows average tumor sizes in xenograft models. MOLM-3 leukemia cells were inoculated s.c. to athymic mice. Tumor mice were divided into groups with similar average tumor sizes when they reached about 120 mm³. 6 mice were treated with a single i.v. 10 mg/kg dose of lintuzumab antibody, 6 mice were treated with a single i.v. 10 mg/kg dose of lintuzumab-exatecan ADC LNExM, 6 mice were treated with a single i.v. 10 mg/kg dose of lintuzumab-exatecan ADC LNExM, and 6 mice were treated with vehicle only. Tumor growth was inhibited the most in the LNExM-treated mice, where 5 mice out of 6 were tumor-free at the predetermined end of the study at day 54 after inoculation, while only 2 mice out of 6 were tumor-free at the end of the study with LNDxM treatment. Naked antibody lintuzumab inhibited tumor growth much less than either of the ADCs. Plotting of the average tumor size is stopped at the time of the first death/group due to tumor growth. Error bars show the standard error of the mean (SEM).

FIG. 5A shows in vitro cytotoxicity assays of exatecan ADCs and free exatecan. HER2+ SK-BR-3 breast cancer cells were incubated with trastuzumab-DXd ADC TRExM, trastuzumab-exatecan ADC TREgM, trastuzumab-exatecan ADC TREbM, trastuzumab-exatecan ADC TREnM, and free exatecan payload (provided as mesylate salt). In the figure it can be seen that TREgM (IC50=0.14 nM) and TREbM (IC50=0.15 nM) had higher cytotoxic activity than TRExM (IC50=0.31 nM), while TREnM (IC50=0.55 nM) had lower activity than TRExM.

FIG. 5B shows in vitro cytotoxicity assays of exatecan ADC, free exatecan, free DXd and trastuzumab. HER2+ SK-BR-3 breast cancer cells were incubated with trastuzumab-deruxtecan ADC TRDxM, free DXd payload and trastuzumab (exatecan is shown for reference). The figure shows that free exatecan (IC50=0.87 nM) had higher cytotoxic activity than free DXd (IC50=4.3 nM), while TRDxM (IC50=0.37 nM) had higher activity than the free payloads, and naked antibody trastuzumab had very low cytotoxic activity against SK-BR-3 cells. Error bars, SEM. Results are from two biological assays.

FIG. 6A shows in vitro cytotoxicity assays of exatecan ADCs and free exatecan. HER2+ HCC1954 breast cancer cells were incubated with trastuzumab-DXd ADC TRExM, trastuzumab-exatecan ADC TREgM, trastuzumab-exatecan ADC TREbM, trastuzumab-exatecan ADC TREnM, and free exatecan payload (provided as mesylate salt). In the figure it can be seen that TREgM, TREbM and TRExM had comparable cytotoxic activity with IC50 between 0.6-0.7 nM and maximum efficacy at about 50% viability, while TREnM had lower activity with maximum efficacy at 76% viability.

FIG. 6B shows in vitro cytotoxicity assays of exatecan ADC, free exatecan, free DXd and trastuzumab. HER2+ HCC1954 breast cancer cells were incubated with trastuzumab-deruxtecan ADC TRDxM, free DXd payload and trastuzumab (exatecan is shown for reference). The figure shows that free exatecan and DXd had comparable activity with IC50 between 5-8 nM. TRDxM had comparable activity to TREgM, TREbM and TRExM, and naked antibody trastuzumab had no cytotoxic activity against HCC1954 cells. Error bars, SEM. Results are from two biological assays.

FIG. 7A shows in vitro cytotoxicity assays of exatecan ADCs and free exatecan. HER2+ JIMT-1 breast cancer cells were incubated with trastuzumab-DXd ADC TRExM, trastuzumab-exatecan ADC TREgM, trastuzumab-exatecan ADC TREbM, trastuzumab-exatecan ADC TREnM, and free exatecan payload (provided as mesylate salt). In the figure it can be seen that TREbM (IC50=31 nM and maximum efficacy at 25% viability) had higher cytotoxic activity than either TREgM or TRExM, which were comparable (IC50 values could not be determined at the concentrations tested), while TREnM had the lowest activity with maximum efficacy at over 90% viability.

FIG. 7B shows in vitro cytotoxicity assays of exatecan ADC, free exatecan, free DXd and trastuzumab. HER2+ JIMT-1 breast cancer cells were incubated with trastuzumab-deruxtecan ADC TRDxM, free DXd payload and trastuzumab (exatecan is shown for reference). The figure shows that that free exatecan (IC50=3.1 nM) had higher cytotoxic activity than free DXd (IC50=18 nM), TRDxM had comparable activity to TREXM, and naked antibody trastuzumab had very low cytotoxic activity against JIMT-1 cells. Error bars, SD.

FIG. 8A shows in vitro cytotoxicity assays of exatecan ADCs and free exatecan. HER2+ SKOV-3 ovarian cancer cells were incubated with trastuzumab-DXd ADC TRExM, trastuzumab-exatecan ADC TREgM, trastuzumab-exatecan ADC TREbM, trastuzumab-exatecan ADC TREnM, and free exatecan payload (provided as mesylate salt). In the figure it can be seen that TREgM (IC50=0.94 nM) and TREbM (IC50=0.56 nM) had comparable cytotoxic activity with maximum efficacy between 60-70% viability, while TREnM had lower activity with maximum efficacy at over 80% viability. TRExM had lower activity: its IC50 value could not be determined at the concentrations tested, but its maximum efficacy was at below 50% viability.

FIG. 8B shows in vitro cytotoxicity assays of exatecan ADC, free exatecan, free DXd and trastuzumab. HER2+ SKOV-3 ovarian cancer cells were incubated with trastuzumab-deruxtecan ADC TRDxM, free dDXd payload and trastuzumab (exatecan is shown for reference). The figure shows that that free exatecan (IC50=4.7 nM) had higher cytotoxic activity than free DXd (IC50=9.5 nM), TRDxM had comparable activity to TREXM, and naked antibody trastuzumab had very low cytotoxic activity against SKOV-3 cells. Error bars, SD.

FIG. 9A shows MALDI-TOF mass spectrometric analysis of the reduced light chain (LC, as [LC+H]⁺ ion) and heavy chain fragments (HC, as [HC+2H]²⁺ ion, showing both G0F and G1F glycoforms) of trastuzumab. y-axis shows relative signal intensity and x-axis shows m/z.

FIG. 9B shows MALDI-TOF mass spectrometric analysis of the reduced light chain (LC, as [LC+H]⁺ ion) and heavy chain fragments (HC, as [HC+2H]²⁺ ion, showing both G0F and G1F glycoforms) of TREnM. y-axis shows relative signal intensity and x-axis shows m/z. The observed changes in m/z (δ) correspond to the conjugated linker-payload structures after stabilization of the maleimides by hydrolysis (+18 mass). ADC was DAR8 according to the analysis.

FIG. 9C shows MALDI-TOF mass spectrometric analysis of the reduced light chain (LC, as [LC+H]⁺ ion) and heavy chain fragments (HC, as [HC+2H]²⁺ ion, showing both G0F and G1F glycoforms) of TREgM. y-axis shows relative signal intensity and x-axis shows m/z. The observed changes in m/z (δ) correspond to the conjugated linker-payload structures after stabilization of the maleimides by hydrolysis (+18 mass). ADC was DAR8 according to the analysis.

FIG. 9D shows MALDI-TOF mass spectrometric analysis of the reduced light chain (LC, as [LC+H]⁺ ion) and heavy chain fragments (HC, as [HC+2H]²⁺ ion, showing both G0F and G1F glycoforms) of TREbM. y-axis shows relative signal intensity and x-axis shows m/z. The observed changes in m/z (δ) correspond to the conjugated linker-payload structures after stabilization of the maleimides by hydrolysis (+18 mass). ADC was DAR8 according to the analysis.

### DETAILED DESCRIPTION

A linker-payload conjugate of Formula I or Formula IB is disclosed:

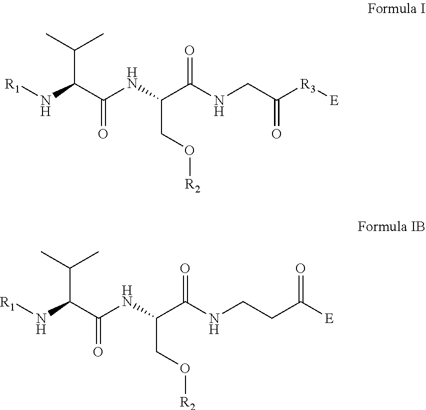

wherein R₁ is a spacer group selected from the group consisting of a maleimidoacetyl, maleimidoacetyl-β-alanyl, and a C₂-C₈ acyl group comprising a bioorthogonal conjugation group; R₂ is selected from the group consisting of a saccharide, a phosphate ester, a sulfate ester, a phosphodiester and a phosphonate; R₃ is a spacer group or absent, wherein the spacer group is selected from the group consisting of a prodrug group and —NH—CH₂—O—CH₂—C(O)—; and E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group.

In this context, the term “linker” should be understood as referring to the moiety or portion of a molecule represented by Formulas I, IB and III that does not comprise E and T and R₃ is absent, or as referring to the moiety or portion of a molecule represented by Formulas I and III that does not comprise E and T and R₃ is present.

In this context, the term “payload” or “payload molecule” may be understood as referring to the exatecan and/or the camptothecin analogue comprising a primary or a secondary amino group.

The bioorthogonal conjugation group may be any group capable of chemically reacting inside a living system without interfering with native biochemical processes of the living system. The bioorthogonal conjugation group may be any group capable of chemically reacting with a suitable group of e.g. a targeting unit, such as an antibody. Various biorthogonal conjugation groups are currently available.

In an embodiment, the bioorthogonal conjugation group is selected from the group consisting of an azidyl, alkynyl, cycloalkynyl, triazolyl, maleimidyl, thiol, succinimidyl, alkenyl, ether, thioether, —CHO, ketone, hydroxylaminyl, hemiacetal, acetal, phosphinyl, tetrazinyl, cyclooctenyl, nitronyl, isoxazolinyl, nitrile oxide, tetrazolyl, pyrazolinyl and quadricyclanyl.

As skilled person will understand, when R₃ is present, the amino group of E together with a carbonyl group of R₃ forms an amide group. When R₃ is absent, the amino group of E together with the carbonyl group to which E is bonded forms an amide group.

As skilled person will also understand, when E is exatecan, the (primary) amino group of exatecan together with a carbonyl group of R₃ forms an amide group. When E is a camptothecin analogue comprising a primary or a secondary amino group, the primary or secondary amino group of the camptothecin analogue together with a carbonyl group of R₃ or, when R₃ is absent, the primary or secondary amino group of the camptothecin analogue together with the carbonyl group to which the primary or secondary amino group of the camptothecin analogue is bonded forms an amide group.

In an embodiment, the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

In an embodiment, the linked-payload conjugate is represented by any one of

Formulas la to If; or the the linked-payload conjugate is represented by any one of Formulas Ia to IBf

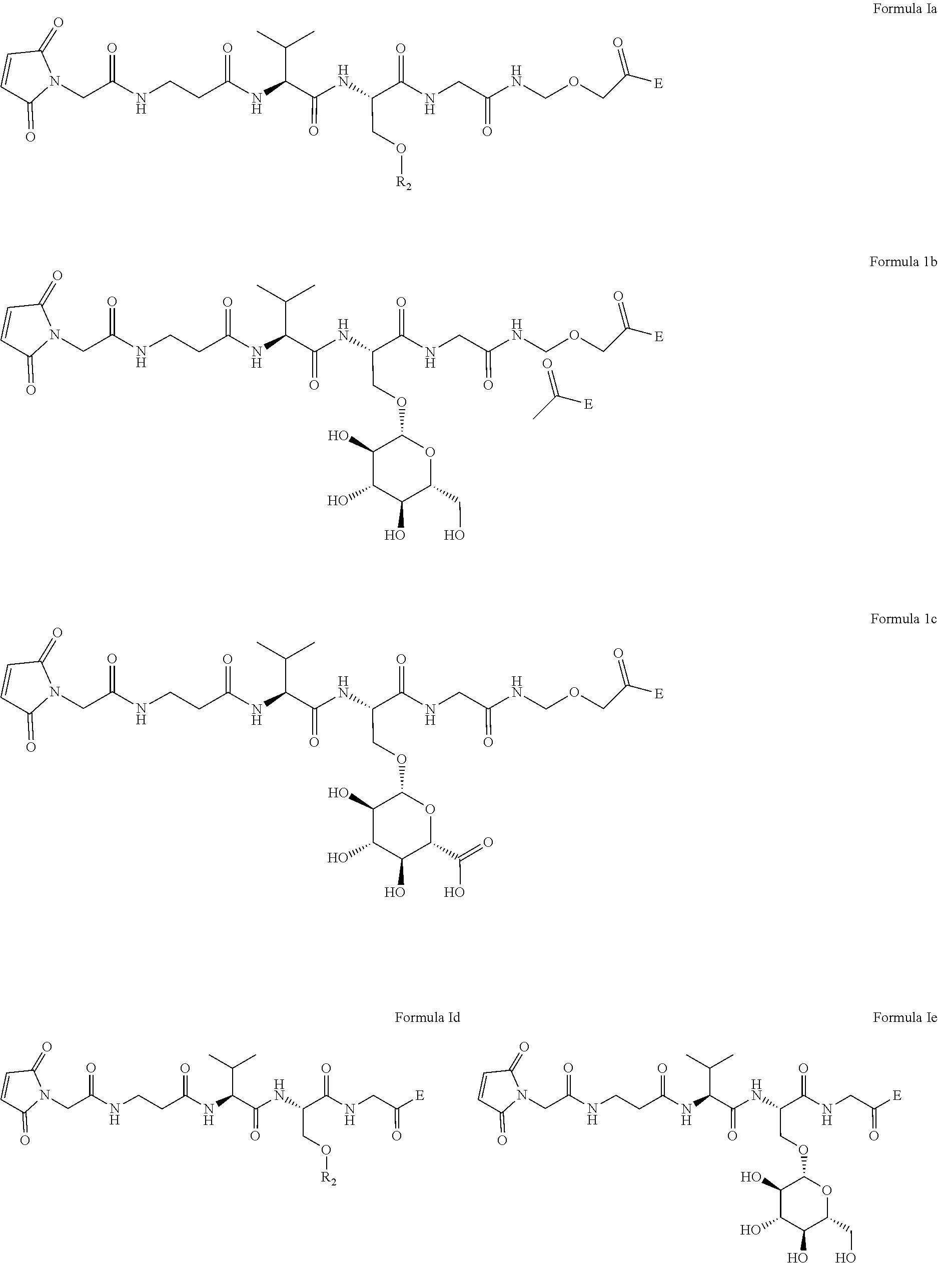

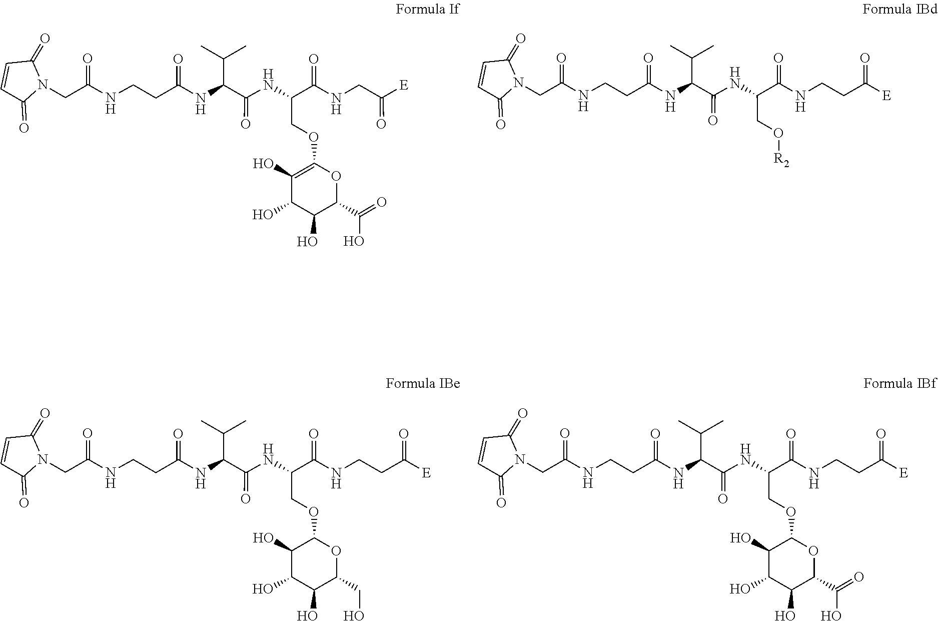

wherein R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification; and E is selected from the group consisting of exatecan and a camptothecin analogue comprising a primary or a secondary amino group, wherein the amino group of E together with the carbonyl group to which E is bonded forms an amide group.

In an embodiment, the linker-payload conjugate is represented by any one of Formulas IIa to IIf, or the linker-payload conjugate is represented by any one of Formulas IIa to IIBf

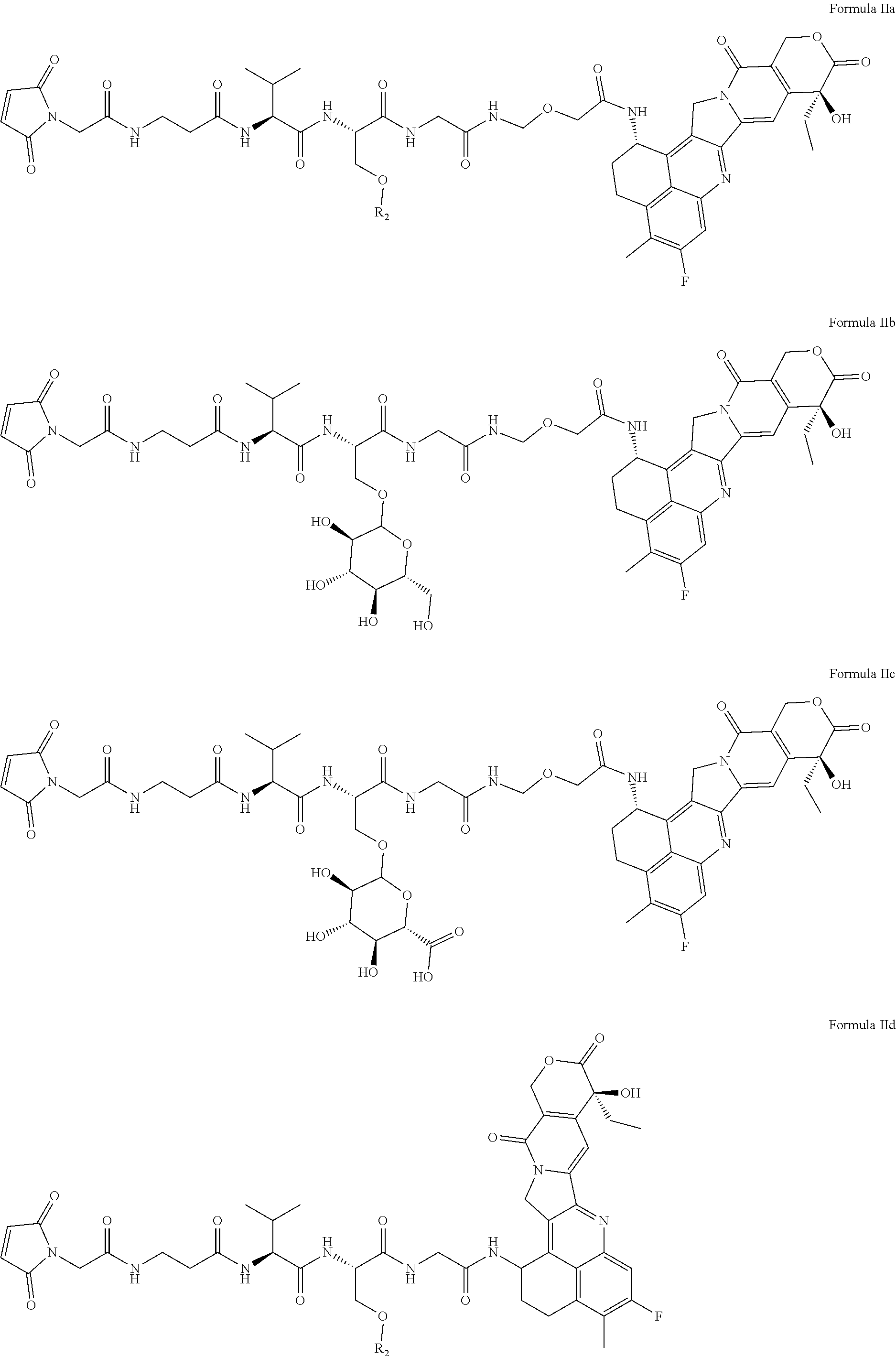

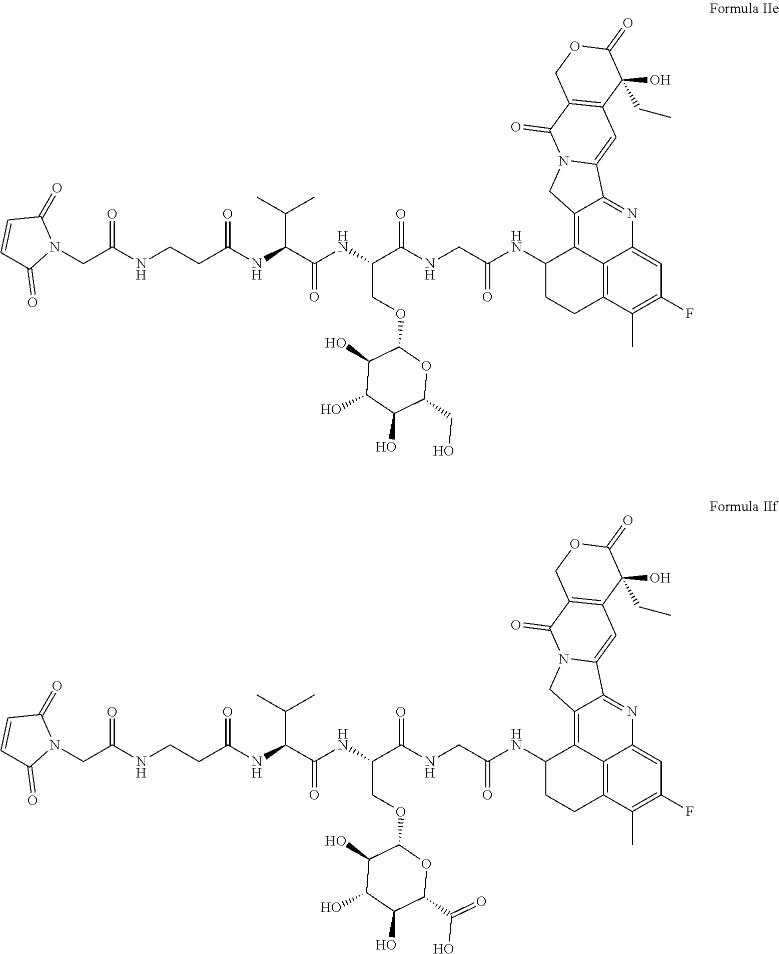

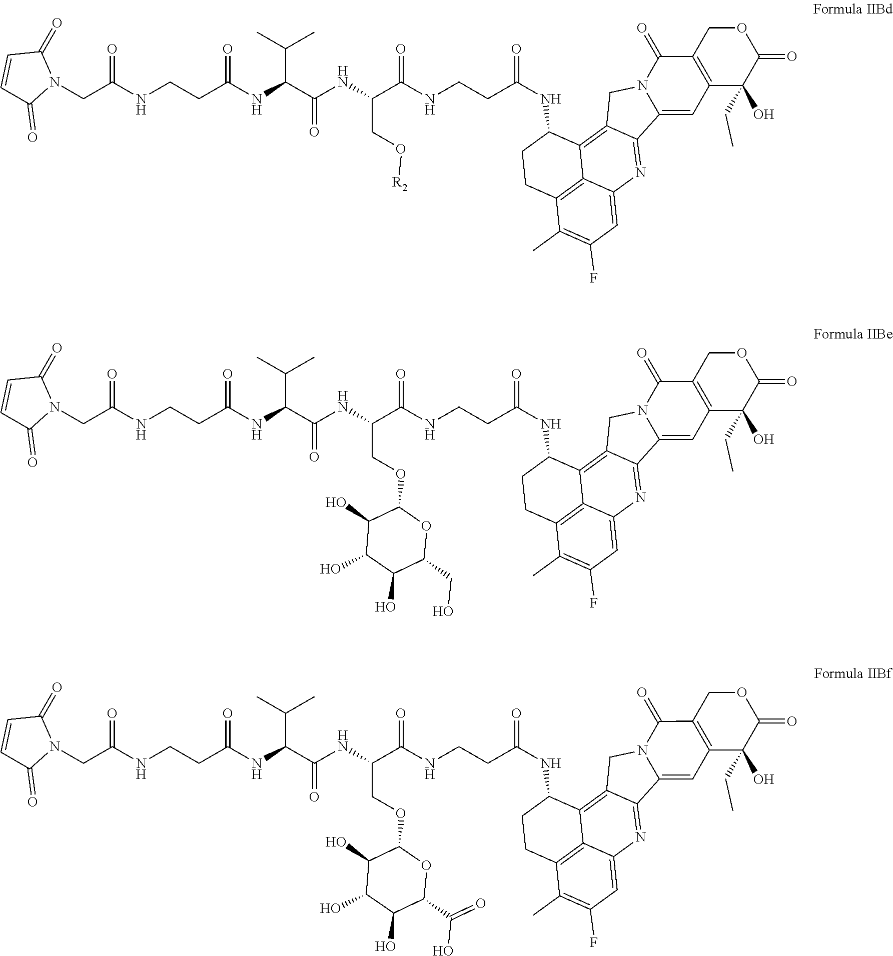

wherein R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

A targeting unit-linker-payload conjugate is also disclosed. The targeting unit-linker-payload conjugate may be represented by Formula III or Formula IIIB

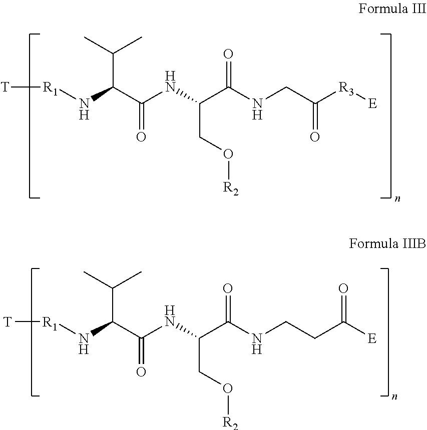

wherein T is a targeting unit; R₁ is a spacer group selected from the group consisting of

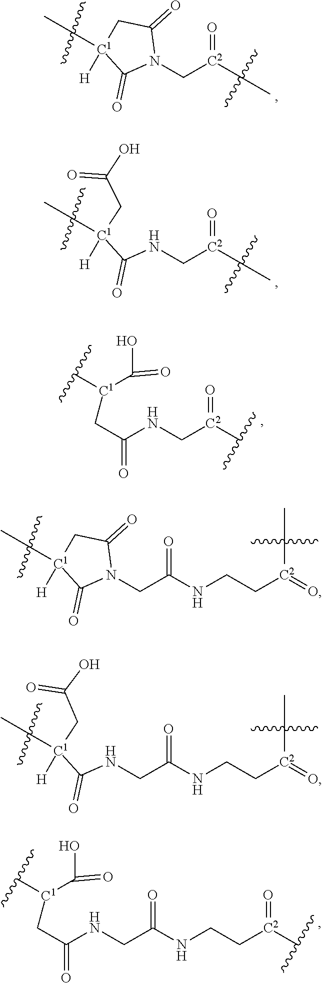

and a C₂-C₈ acyl group comprising a radical of a bioorthogonal conjugation group; R₂ is selected from the group consisting of a saccharide, a phosphate ester, a sulfate ester, a phosphodiester and a phosphonate; R₃ is a spacer group or absent, wherein the spacer group is selected from the group consisting of a prodrug group and —NH—CH₂—O—CH₂—C(O)—; E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group; and n≥1.

In an embodiment of the targeting unit-linker-payload conjugate, the radical of the bioorthogonal conjugation group is derived from a group selected from the group consisting of an azidyl, alkynyl, triazolyl, maleimidyl, thiol, succinimidyl, alkenyl, ether, thioether, —CHO, ketone, hydroxylaminyl, hemiacetal, acetal, phosphinyl, tetrazinyl, cyclooctenyl, nitronyl, isoxazolinyl, nitrile oxide, tetrazolyl, pyrazolinyl and quadricyclanyl.

In an embodiment of the targeting unit-linker-payload conjugate, the radical of the bioorthogonal conjugation group is selected from the group consisting of

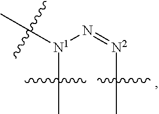

wherein N¹ and N², together with two carbons of the targeting unit to which they are attached to, form a 1,2,3-triazole, and wherein N¹ is also covalently attached to a carbon of the C₂-C₈ acyl group;

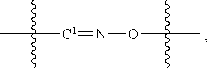

wherein one of C¹ and O is covalently attached to a carbon of the C₂-C₈ acyl group and the other one of C¹ and O is covalently attached to the targeting unit;

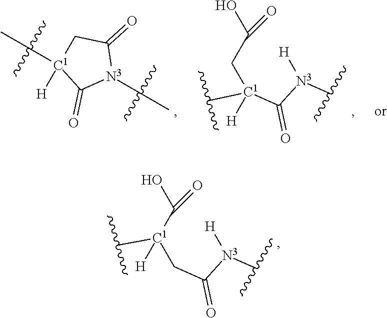

wherein N³ is covalently attached to a carbon of the C₂-C₈ acyl group and C¹ is covalently attached to the targeting unit;

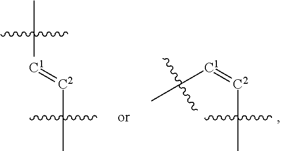

wherein one of C¹ and C² is covalently attached to a carbon of the C₂-C₈ acyl group and the other one of C¹ and C² is covalently attached to the targeting unit;

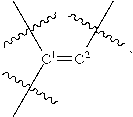

wherein C¹ and C², together with two nitrogens of the targeting unit they are attached to, form a 1,2,3-triazole, and C¹ is also covalently attached to a carbon of the C₂-C₈ acyl group;

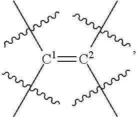

wherein C¹ and C², together with two nitrogens of the targeting unit they are attached to, form a 1,2,3-triazole, and C¹ and C² being part of a cycloalkenyl such as cyclooctenyl or (10Z)-tricyclo [10.4.0.04,9] hexadeca-1(16),4,6,8,10,12, 14-heptaen-2-yl, which is covalently attached to a carbon of the C²-C⁸ acyl group or to a —O—, —S—, —O₂C—, —O₂CNH—, or —NHCO₂— linker, which is covalently attached to the C₂-C₈ acyl group, or
wherein C¹ and C², together with an oxygen and a carbon of the targeting unit they are attached to, form a N-alkyl isoxazoline, and C¹ and C² being part of a cycloalkenyl such as cyclooctenyl or (10Z)-tricyclo [10.4.0.04,9] hexadeca-1(16),4,6,8,10,12,14-heptaen-2-yl, which is covalently attached to a carbon of the C₂-C₈ acyl group or to a —O—, —S—, —O₂C—, —O₂CNH—, or —NHCO₂— linker, which is covalently attached to the C₂-C₈ acyl group;

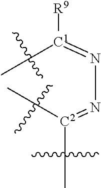

or its tautomer form, wherein C¹ and C² are covalently attached to two carbons of the targeting unit, and wherein C² is also covalently attached to a carbon of the C₂-C₈ acyl group or to a —O— or —S— linker, which is covalently attached to the C₂-C₈ acyl group, wherein R⁹ is H, C₁₋₅-alkyl, or 2-pyridyl;

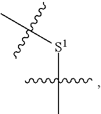

wherein S¹ is covalently attached to the targeting unit and a carbon of the C₂-C₈ acyl group;

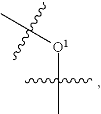

wherein O¹ is covalently attached to the targeting unit and a carbon of the C₂-C₈ acyl group;

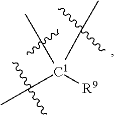

wherein C¹ is covalently attached to a carbon of the C₂-C₈ acyl group and to two oxygens of the targeting unit, and R⁹ is H, or a C₁₋₅-alkyl;

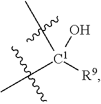

wherein C¹ is covalently attached to a carbon of the C₂-C₈ acyl group and to an oxygen of the targeting unit, and R⁹ is H, or a C₁₋₅-alkyl;

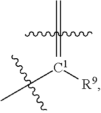

wherein C¹ is covalently attached to a carbon of the C₂-C₈ acyl group and C¹ is also covalently attached, with the double bond, to a nitrogen of the targeting unit, and R⁹ is H, or a C₁₋₅-alkyl;

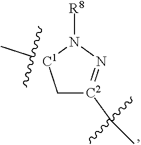

wherein C¹ is covalently attached to a carbon of the C₂-C₈ acyl group or to to a —O—, —CH₂O— or —CH₂OPhCH₂— linker, which is covalently attached to the C₂-C₈ acyl group, and C² is covalently attached to the targeting unit; or wherein C¹ is covalently attached to the targeting unit and C² is covalently attached to a carbon of the C₂-C₈ acyl group or to to a —PhCO₂— linker, which is covalently attached to the C₂-C₈ acyl group, wherein R⁸ is C₁₋₅-alkyl or an optionally substituted phenyl;

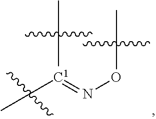

wherein C¹ and O, together with two carbons of the targeting unit they are attached to, form an isoxazoline such as a 2-isoxazoline, 3-isoxazoline, or 4-isoxazoline, and wherein C¹ is also covalently attached to a carbon of the C₂-C₈ acyl group, or wherein C¹ and O, together with two carbons of a cycloalkenyl, such as 2,5-norbornadien-2-yl, form an isoxazoline such as a 2-isoxazoline, 3-isoxazoline, or 4-isoxazoline, wherein the isooxazoline being covalently attached to a carbon of the C₂-C₈ acyl group or to a —O— or —OCH₂— linker, which is covalently attached to the C₂-C₈ acyl group; and

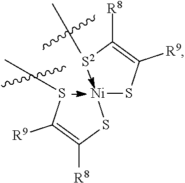

wherein S¹ and S² are covalently attached to the targeting unit, R⁸ is phenyl, and R⁹ is a linker group, such as —PhCONH—, which is covalently attached to the C₂-C₈ acyl group, or one of R⁹ is phenyl and the other one of R⁹ is a linker group, such as —PhCONH—, which is covalently attached to the C₂-C₈ acyl group.

In an embodiment, the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

In an embodiment, the targeting unit-linker-payload conjugate is represented by Formula IV or Formula IVB

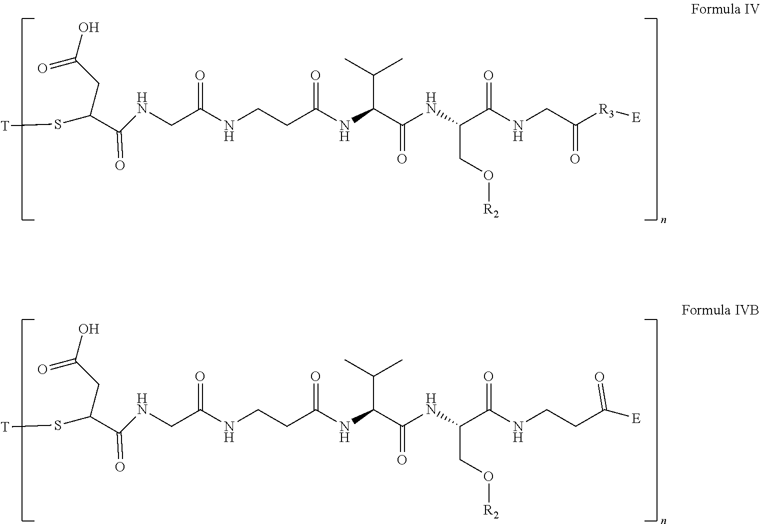

wherein T is an antibody; n is in the range of 1 to about 20; R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification; R₃ is a spacer group or absent, wherein the spacer group is selected from the group consisting of a prodrug group and —NH—CH₂—O—CH₂—C (O)—; E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group.

In an embodiment, the targeting unit-linker-payload conjugate is represented by any one of Formulas IVa to IVf, or the targeting unit-linker-payload conjugate is represented by any one of Formulas IVa to IVBf

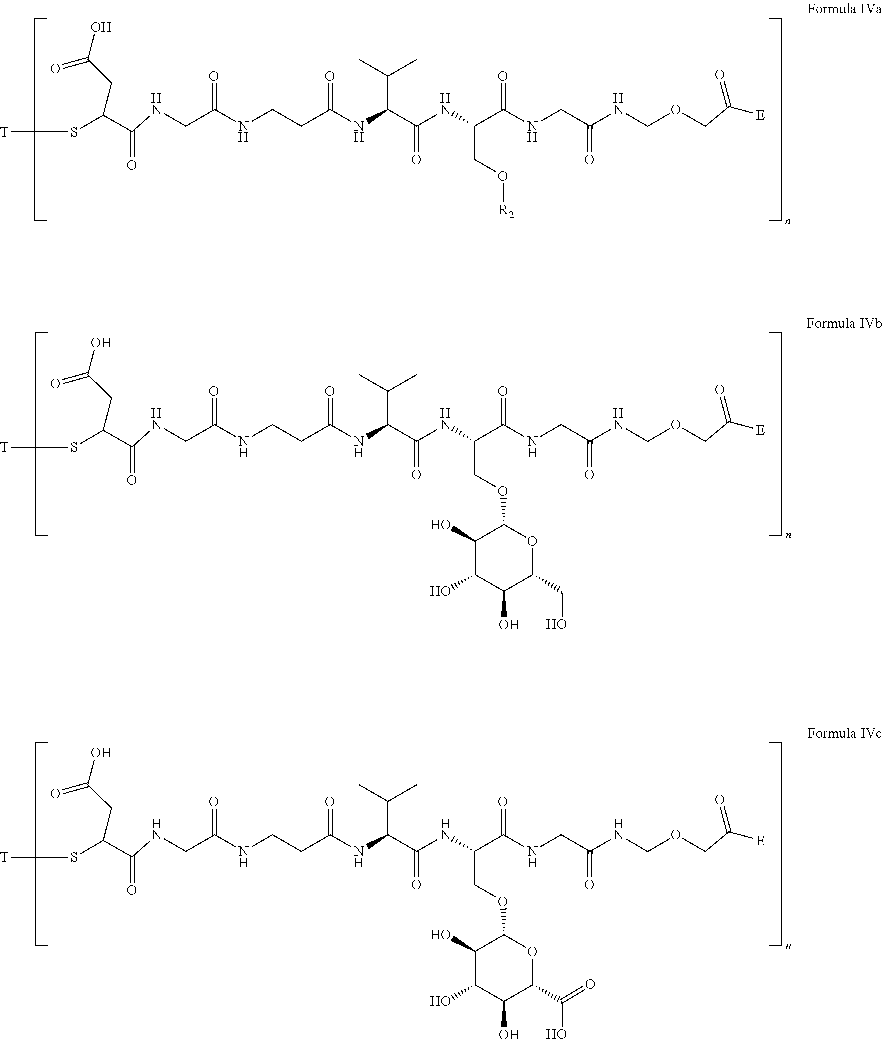

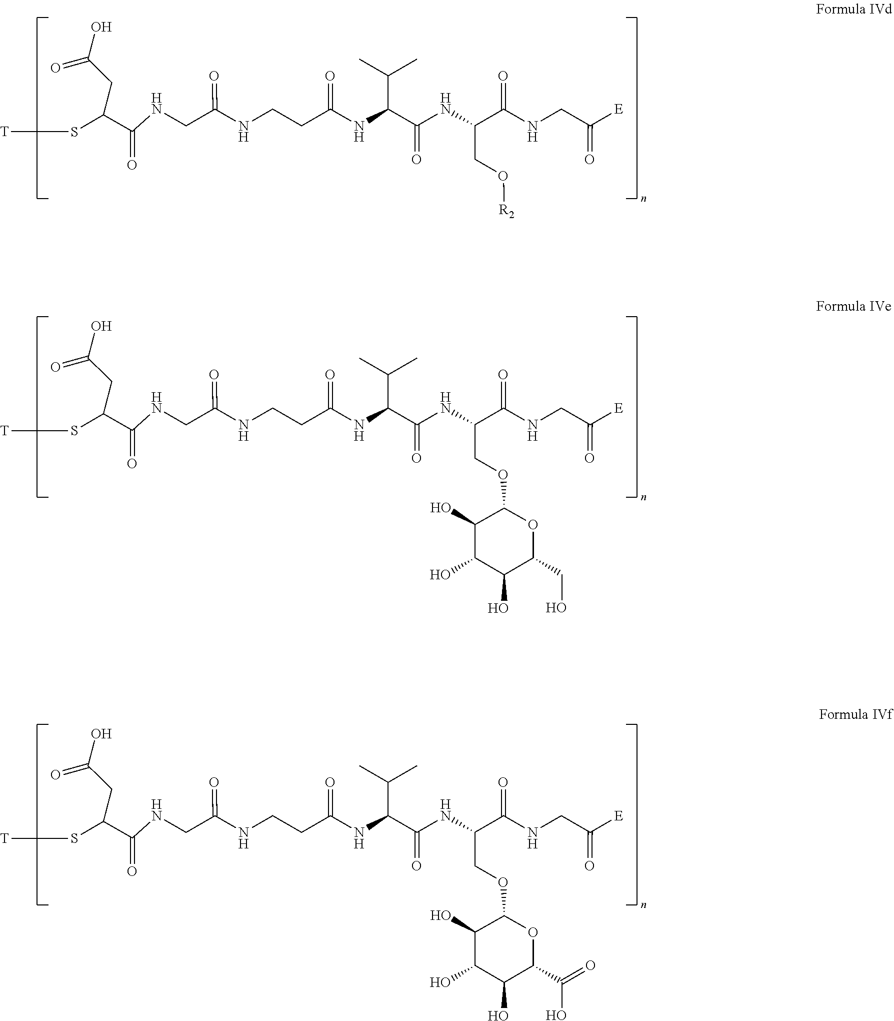

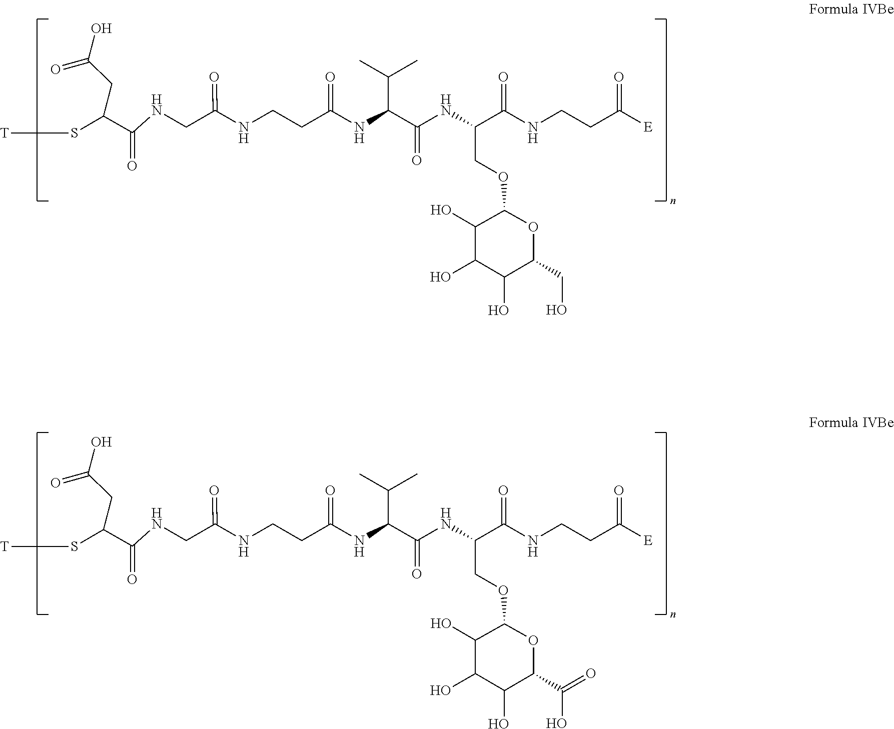

wherein T is an antibody; n is in the range of 1 and about 20; R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification; E is selected from the group consisting of exatecan and a camptothecin analogue comprising a primary or a secondary amino group, wherein the amino group of E together with the carbonyl group to which E is bonded forms an amide group.

In an embodiment, the targeting unit-linker-payload conjugate is represented by any one of Formulas Va to Vf, or the targeting unit-linker-payload conjugate is represented by any one of Formulas Va to VBf

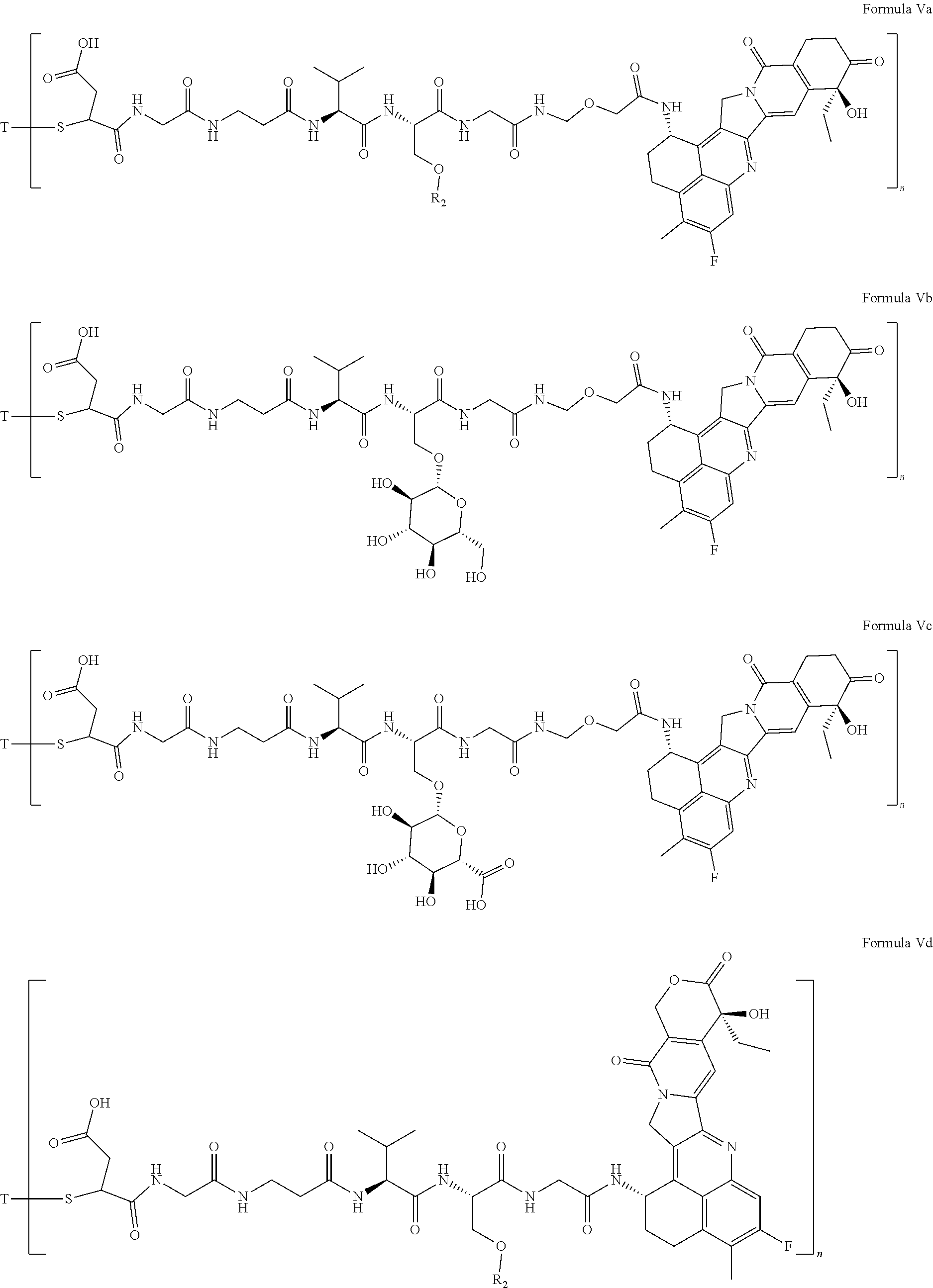

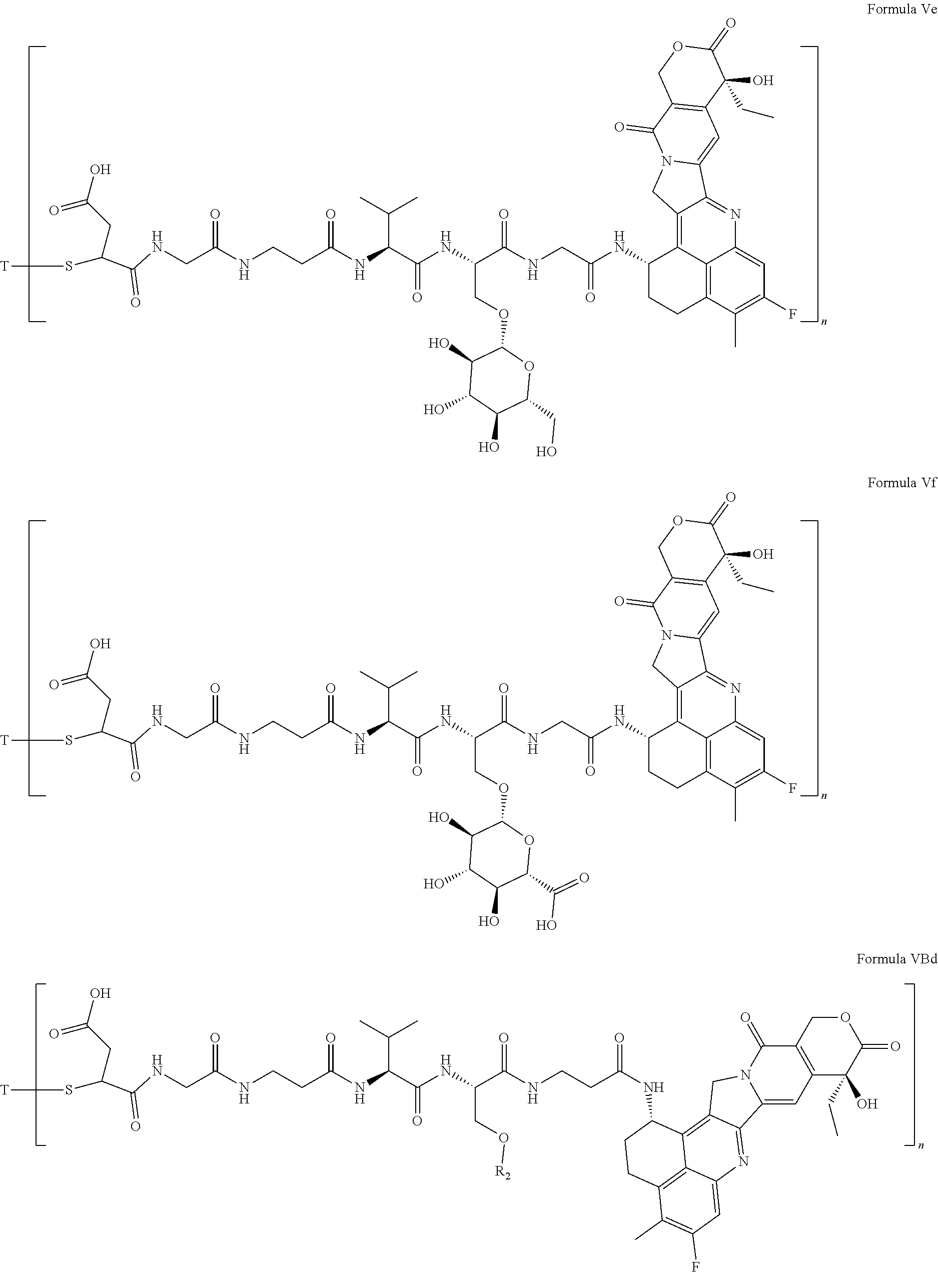

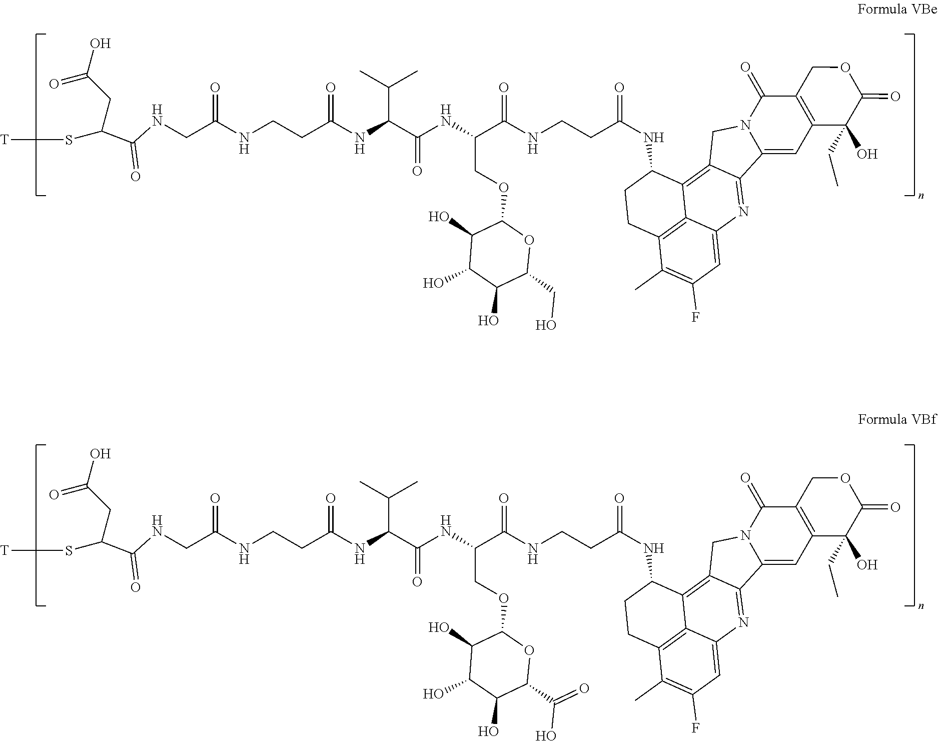

wherein T is an antibody; n is in the range of 1 and about 20; R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

In an embodiment, n is in the range of 1 to about 20, or in the range of 1 to about 15, or in the range of 1 to about 10, or in the range of 2 to 10, or in the range of 2 to 6, or in the range of 2 to 5, or in the range of 2 to 4; or n is 1, 2, 3, 4, 5, 6, 7, 8, 9, 10, 11, 12, 13, 14, 15, 16, 17, 18, 19, or 20.

In an embodiment, n is in the range of 3 to about 20, or in the range of 3 to about 15, or in the range of 3 to about 10, or in the range of 3 to about 9, or in the range of 3 to about 8, or in the range of 3 to about 7, or in the range of 3 to about 6, or in the range of 3 to 5, or in the range of 3 to 4.

In an embodiment, n is in the range of 4 to about 20, or in the range of 4 to about 15, or in the range of 4 to about 10, or in the range of 4 to about 9, or in the range of 4 to about 8, or in the range of 4 to about 7, or in the range of 4 to about 6, or in the range of 4 to 5.

In an embodiment, n is 5.

In an embodiment, n is 6.

In an embodiment, n is 7.

In an embodiment, n is 8.

In an embodiment, n is 9.

In an embodiment, the targeting unit-linker-payload conjugate is represented by formula VBe, wherein T is an antibody, and n is in the range of 1 to about 20; or T is an antibody, and n is about 8; or Tis trastuzumab, and n is about 8.

A skilled person will recognize that the linker-payload conjugate moiety linked to targeting unit or spacer as represented in Formula III is derived from and thereby essentially the same as represented by Formula I. In the targeting unit-linker-payload conjugate, the targeting unit, T, and the payload, E, have thus reacted at the two ends of the linker (without R₃), or reacted with the R₁ and R₃ of the linker, respectively. Using the linkers according to the present disclosure, one or more payload molecules can be introduced to a targeting unit. Likewise, a skilled person will recognize that the linker-payload conjugate moiety linked to targeting unit or spacer as represented in Formula IIIB is derived from and thereby essentially the same as represented by Formula IB.

In an embodiment, the linker-payload conjugate may be represented as of Formula T-R₁-L-R₃-E, wherein T, R₁, R₃, and E are as represented in Formulas I and III or in Formulas IB and IIIB and L is the linker of the present disclosure.

In an embodiment, R₂ is selected from the group consisting of a saccharide, phosphate ester, sulfate ester, a phosphodiester and a phosphonate.

In an embodiment, R₂ is a saccharide.

The term “saccharide” should be understood as referring to a single simple sugar moiety or monosaccharide or a derivative thereof, as well as a combination of two or more single sugar moieties or monosaccharides covalently linked to form a disaccharide, oligosaccharide, or polysaccharide.

In this context, the term “saccharide” may be understood as referring to a radical of a saccharide. Therefore any references to (individual) saccharides in this specification may also be understood as referring to radicals of the saccharides.

The term “monosaccharide” should be understood to include trioses, tetroses, pentoses, hexoses, heptoses, octoses or nonoses. One or several of the hydroxyl groups in the chemical structure can be replaced with other groups such as hydrogen, amino, amine, acylamido, acetylamido, halogen, mercapto, acyl, acetyl, phosphate or sulphate ester, and the like; and the saccharides can also comprise other functional groups such as carboxyl, carbonyl, hemiacetal, acetal and thio groups. A monosaccharide can selected from the group including, but not limited to, simple aldoses such as glyceraldehyde, erythrose, threose, ribose, arabinose, xylose, lyxose, allose, altrose, glucose, mannose, gulose, idose, galactose, talose and mannoheptulose; simple ketoses such as dihydroxyacetone, erythrulose, ribulose, xylulose, psicose, fructose, sorbose, tagatose and sedoheptulose; deoxysugars such as fucose, 2-deoxyglucose, 2-deoxyribose and rhamnose; sialic acids such as ketodeoxynonulosonic acid, N-acetylneuraminic acid and 9-O-acetyl-N-acetylneuraminic acid; uronic acids such as glucuronic acid, galacturonic acid and iduronic acid; amino sugars such as 2-amino-2-deoxygalactose and 2-amino-2-deoxyglucose; acylamino sugars such as 2-acetamido-2-deoxygalactose, 2-acetamido-2-deoxyglucose and N-glycolylneuraminic acid; phosphorylated and sulphated sugars such as 6-phosphomannose, 6-sulpho-N-acetylglucosamine and 3-sulphogalactose; and derivatives and modifications thereof. The monosaccharide can also be a non-reducing carbohydrate such as inositol or alditol or their derivative.

Saccharides and monosaccharides according to the present disclosure may be in D- or L-configuration; in open-chain, pyranose or furanose form; α or β anomer; and any combination thereof.

The term “oligosaccharide” may be understood as referring to saccharides composed of two or several monosaccharides linked together by glycosidic bonds having a degree of polymerization in the range of from 2 to about 20. The term “oligosaccharide” should be understood as referring to hetero-and homopolymers that can be either branched, linear or cyclical. In an embodiment, the oligosaccharide has a reducing end and a non-reducing end, whether or not the saccharide at the reducing end is in fact a reducing sugar.

The term “disaccharide” may be understood as referring to an oligosaccharide composed of two monosaccharides linked together by a glycosidic bond. Examples of disaccharides include, but are not limited to, lactose, N-acetyllactosamine, galactobiose, maltose, isomaltose and cellobiose.

The term “trisaccharide” may be understood as referring to a saccharide composed of three monosaccharides linked together by glycosidic bonds. Examples of trisaccharides include, but are not limited to, maltotriose, sialyllactose, globotriose, lacto-N-triose and gangliotriose.

In an embodiment, the saccharide is a monosaccharide, a disaccharide, a trisaccharide or an oligosaccharide.

In an embodiment, the saccharide comprises β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, neuraminic acid or any analogue or modification thereof.

In an embodiment, the saccharide consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, neuraminic acid or any analogue or modification thereof.

In an embodiment, the saccharide consists of β-D-glucose, N-acetyl-β-D-glucosamine, β-D-glucuronic acid or α-L-fucose.

In an embodiment, the saccharide comprises β-D-glucose.

In an embodiment, the saccharide consists of β-D-glucose.

In an embodiment, the modification is sulfate, phosphate, carboxyl, amino, or O-acetyl modification of the monosaccharide.

In the context of the saccharide, the term “analogue” or “being analogous to” should be understood so that the analogue or the analogous monosaccharide is cleavable by the same enzyme than the monosaccharide to which it is analogous to.

The term “modification” or “modification of a monosaccharide” should be understood so that the modification is a covalent modification of a monosaccharide resulting from substitution of a functional group or an atom of the monosaccharide.

In an embodiment, the modification is selected from the group of sulfate, phosphate, carboxyl, amino, and O-acetyl modification.

In an embodiment, R₂ is cleavable by an enzyme.

In an embodiment, R₂ is cleavable by an enzyme, for example, an intracellular enzyme, a lysosomal enzyme or a cytoplasmic enzyme.

In an embodiment, the cleavable hydrophilic group R₂ is a saccharide and cleavable by an enzyme.

In an embodiment, the saccharide is β-D-glucose, N-acetyl-β-D-glucosamine, β-D-glucuronic acid or α-L-fucose.

In an embodiment, the saccharide is β-D-glucose.

In an embodiment, the enzyme is an intracellular enzyme, a lysosomal enzyme or a cytoplasmic enzyme.

In an embodiment, the intracellular enzyme is a glucosidase, a hexosaminidase, an N-acetylglucosaminidase, a glucuronidase or a fucosidase.

In an embodiment, the lysosomal enzyme is a glucosidase, a hexosaminidase, an N-acetylglucosaminidase, a glucuronidase or a fucosidase.

In an embodiment, the lysosomal enzyme is β-glucosidase.

In an embodiment, the cytoplasmic enzyme is a glucosidase, a hexosaminidase, an N-acetylglucosaminidase, a glucuronidase or a fucosidase.

In an embodiment, the saccharide such as β-D-glucose is cleavable by a lysosomal or an intracellular enzyme. This embodiment has the utility that lysosomal or intracellular enzymes may remove the saccharide inside a cell. A skilled person is capable of selecting a saccharide that is cleavable by a lysosomal or an intracellular enzyme based on biochemical literature; various such enzymes having different specificities are known.

In an embodiment, the lysosomal or intracellular enzyme is capable of removing the entire saccharide inside a cell.

In an embodiment, one or more of the glycosidic bonds of the saccharide are essentially stable in neutral pH and/or in serum.

In an embodiment, all glycosidic bonds of the saccharide are essentially stable in neutral pH and/or in serum.

In an embodiment, one or more of the glycosidic bonds of the saccharide are cleavable in tumor microenvironment outside a cell. This embodiment has the added utility that the saccharide may be removed more efficiently inside a tumor than in normal tissue and the molecule may be more efficiently taken up by cancer cells than by normal cells.

In an embodiment, the saccharide protects the linker from cleavage by a peptidase before the saccharide is cleaved by a glycosidase enzyme.

In an embodiment, the saccharide is β-D-glucose that protects the linker from cleavage by a peptidase before the saccharide is cleaved by β-glucosidase.

In an embodiment, the saccharide protects the linker from cleavage by cathepsin before the saccharide is cleaved by a glycosidase enzyme.

In an embodiment, the saccharide is β-D-glucose that protects the linker from cleavage by cathepsin before the saccharide is cleaved by β-glucosidase.

In an embodiment, the lysosomal or intracellular enzyme is selected from the group consisting of β-galactosidase, β-hexosaminidase, α-N-acetylgalactosaminidase, β-N-acetylglucosaminidase, β-glucuronidase, α-L-iduronidase, α-galactosidase, α-glucosidase, β-glucosidase, α-mannosidase, β-mannosidase, α-fucosidase, β-xylosidase and neuraminidase.

In an embodiment, the human glycohydrolase is selected from the group consisting of β-galactosidase, β-hexosaminidase, α-N-acetylgalactosaminidase, β-N-acetylglucosaminidase, β-glucuronidase, α-L-iduronidase, α-galactosidase, α-glucosidase, β-glucosidase, α-mannosidase, β-mannosidase, α-fucosidase, β-xylosidase and neuraminidase.

In an embodiment, R₂ is phosphate ester. In this context, the term “phosphate ester” may be understood as referring to a radical of phosphate ester.

In an embodiment, R₂ is sulfate ester. In this context, the term “sulfate ester” may be understood as referring to a radical of sulfate ester.

In an embodiment, R₂ is a phosphodiester. In this context, the term “phosphodiester” may be understood as referring to a radical of phosphodiester.

In an embodiment, the phosphodiester is pyrophosphate, O—P(═O)(OH)—O—P(═O)(OH)₂.

In an embodiment, the phosphodiester is a substituted pyrophosphate selected from the group of O—P(═O)(OH)—O—P(═O)(OH)OR and O—P(═O)(OH)—O—P(═O)(OH)R, wherein R is selected from the group of P(═O)(OH)R, CH₃, an alkyl group and an aryl group. In an embodiment, the alkyl group is CH₂CH₂NH₂. In an embodiment, the aryl group is benzyl.

In an embodiment, R₂ is a phosphonate.

In an embodiment, the phosphonate is bisphosphonate, O—P(═O)(OH)—CH₂—P(═O)(OH)₂.

In an embodiment, the phosphonate is substituted bisphosphonate selected from the group of O—P(═O)(OH)—CH₂—P(═O)(OH)OR and O—P(═O)(OH)—CH₂—P(═O)(OH)R, wherein R is selected from the group of P(═O)(OH)R, CH₃, an alkyl group and an aryl group. In an embodiment, the alkyl group is CH₂CH₂NH₂. In an embodiment, the aryl group is benzyl.

Phosphodiester and bisphosphonate groups according to the present disclosure can be prepared as described in Yates and Fiedler, ACS Chem. Biol. 2016, 11, 1066-1073, and incorporated as protected modified amino acids such as protected phosphodiester-modified or protected bisphosphonate-modified serine building blocks in standard peptide synthesis chemistry to produce the linker moieties according to the present disclosure.

In an embodiment, the cleavable hydrophilic group R₂ is capable of inhibit an endopeptidase from liberating the payload E from the conjugate until R₂ is first cleaved away from the conjugate.

The term “alkyl” should be understood as referring to a straight or branched chain saturated or unsaturated hydrocarbon having the indicated number of carbon atoms (e.g., “C₁-C₈ alkyl” refers to an alkyl group having from 1 to 8 carbon atoms). When the number of carbon atoms is not indicated, the alkyl group has from 1 to 8 carbon atoms. Representative “C₁-C₈ alkyl” groups include (but are not limited to) methyl (Me, CH₃), ethyl (Et, CH₂CH₃), 1-propyl (n-Pr, n-propyl, CH₂CH₂CH₃), 2-propyl (i-Pr, isopropyl, CH(CH₃)₂), 1-butyl (n-Bu, n-butyl, CH₂CH₂CH₂CH₃), 2-methyl-1-propyl (i-Bu, isobutyl, CH₂CH(CH₃)₂), 2-butyl (s-Bu, s-butyl, CH(CH₃)CH₂CH₃), 2-methyl-2-propyl (t-Bu, tert-butyl, C(CH₃)₃), 1-pentyl (n-pentyl, CH₂CH₂CH₂CH₂CH₃), 2-pentyl (CH(CH₃)CH₂CH₂CH₃), 3-pentyl (CH(CH₂CH₃)₂), 2-methyl-2-butyl (C(CH₃)₂CH₂CH₃), 3-methyl-2-butyl (CH(CH₃)CH(CH₃)₂), 3-methyl-1-butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3-hexyl (CH(CH₂CH₃)(CH₂CH₂CH₃)), 2-methyl-2-pentyl (C(CH₃)₂CH₂CH₂CH₃), 3-methyl-2-pentyl (CH(CH₃)CH(CH₃)CH₂CH₃), 4-methyl-2-pentyl (CH(CH₃)CH₂CH(CH₃)₂), 3-methyl-3-pentyl (C(CH₃)(CH₂CH₃)₂), 2-methyl-3-pentyl (CH(CH₂CH₃)CH(CH₃)₂), 2,3-dimethyl-2-butyl (C(CH₃)₂CH(CH₃)₂), and 3,3-dimethyl-2-butyl (CH(CH₃)C(CH₃)₃). An alkyl group can be unsubstituted or substituted with one or more groups including, but not limited to, OH, O(C₁-C₈ alkyl), aryl, COR′, OCOR′, CONH₂, CONHR′, CONR′₂, NHCOR′, SH, SO₂R′, SOR′, OSO₂OH, OPO(OH)₂, halogen, N₃, NH₂, NHR′, NR′₂, NHCO(C₁-C₈ alkyl) or CN, wherein each R′ is independently either H, C₁-C₈ alkyl or aryl. The term “alkyl” should also be understood as referring to an alkylene, a saturated, branched or straight chain or cyclic hydrocarbon radical of 1-18 carbon atoms, and having two monovalent radical centers derived by the removal of two hydrogen atoms from the same or two different carbon atoms of a parent alkane. Typical such alkylenes include (but are not limited to) methylene (CH₂) 1,2-ethyl (CH₂CH₂), 1,3-propyl (CH₂CH₂CH₂), 1,4-butyl (CH₂CH₂CH₂CH₂), and the like. The term “alkyl” should also be understood as referring to arylalkyl and heteroarylalkyl radicals as described below.

The term “arylalkyl” should be understood as referring to an acyclic alkyl radical in which one of the hydrogen atoms bonded to a carbon atom, typically a terminal or sp³ carbon atom, is replaced with an aryl radical. Typical arylalkyl groups include (but are not limited to) benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

The term “heteroarylalkyl” should be understood as referring to an acyclic alkyl radical in which one of the hydrogen atoms bonded to a carbon atom, typically a terminal or sp³ carbon atom, is replaced with a heteroaryl radical. Typical heteroarylalkyl groups include (but are not limited to) 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 ring atoms, typically 1 to 3 heteroatoms selected from N, O, P, and S, with the remainder being carbon atoms. The heteroaryl moiety of the heteroarylalkyl group may be a monocycle having 3 to 7 ring members (2 to 6 carbon atoms) or a bicycle having 7 to 10 ring members (4 to 9 carbon atoms) and 1 to 3 heteroatoms selected from N, O, P, and S, for example: a bicyclo [4,5], [5,5], [5,6], or [6,6] system.

The term “alkynyl” should be understood as referring to a C₂-C₁₈ hydrocarbon containing normal, secondary, tertiary or cyclic carbon atoms with at least one site of unsaturation, i.e., a carbon-carbon, sp triple bond. Examples include, but are not limited to acetylenic (C═CH) and propargyl (CH₂C═CH). The term “alkynyl” should also be understood as referring to an alkynylene, an unsaturated, branched or straight chain or cyclic hydrocarbon radical of 2-18 carbon atoms, and having two monovalent radical centers derived by the removal of two hydrogen atoms from carbon atoms of a parent alkyne. Typical alkynylene radicals include (but are not limited to) acetylene (C═C), propargyl (CH₂C═C), and 4-pentynyl (CH₂CH₂CH₂C═C).

The term “cycloalkynyl” should be understood as referring to a C₂-C₁₈ hydrocarbon containing cyclic carbon atoms with at least one site of unsaturation, i.e., a carbon-carbon, sp triple bond. Cycloalkynyls include substituted cyclooctynyl structures and fused cyclooctynyl structures, i.e. cyclooctynyl groups wherein one or more ring structures are fused to the central cyclooctynyl moiety. Typical cycloalkynyl radicals include (but are not limited to) DBCO, DIBO, BARAC, DIBAC, DIFO, DIFO2, DIFO3 and BCN.

The term “alkenyl” should be understood as referring to a C₂-C₁₈ hydrocarbon containing normal, secondary, tertiary or cyclic carbon atoms with at least one site of unsaturation, i.e., a carbon-carbon, sp² double bond. Examples include, but are not limited to ethylene or vinyl (CH═CH₂), allyl cyclopentenyl (CH₂CH═CH₂), (C₅H₇), and 5-hexenyl (CH₂CH₂CH₂CH₂CH═CH₂). The term “alkenyl” should also be understood as referring to an alkenylene, an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to 1,2-ethylene (CH═CH).

In the context of this specification, the term “substituted”, when used as adjective to “alkyl”, “heteroalkyl”, “cycloalkyl”, “heterocycloalkyl”, “aryl”, “heteroaryl”, “alkylaryl” and the like, indicates that said “alkyl”, “heteroalkyl”, “cycloalkyl”, “heterocycloalkyl”, “aryl”, “alkylaryl” or “heteroaryl” group contains one or more substituents, which may include, but are not limited to, OH, ═O, ═S, ═NRh, ═N—ORh, SH, NH₂, NO₂, NO, N₃, CF₃, CN, OCN, SCN, NCO, NCS, C(O)NH₂, C(O)H, C(O)OH, halogen, Rh, SRh, S(O)Rh, S(O)ORh, S(O)₂Rh, S(O)₂ORh, OS(O)Rh, OS(O)ORh, OS(O)₂Rh, OS(O)₂ORh, OP(O)(ORh)(ORi), P(O)(ORh)(ORi), ORh, NHRi, N(Rh)Ri, +N(Rh)(Ri)Rj, Si(Rh)(Ri)(Rj), C(O)Rh, C(O)ORh, C(O)N(Ri)Rh, OC(O)Rh, OC(O)ORh, OC(O)N(Rh)Ri, N(Ri)C(O)Rh, N(Ri)C(O)ORh, N(Ri)C(O)N(Rj)Rh, and the thio derivatives of these substituents, or a protonated or deprotonated form of any of these substituents, wherein Rh, Ri, and Rj are independently selected from H and optionally substituted C₁₋₁₅ alkyl, C₁₋₁₅ heteroalkyl, C₃₋₁₅ cycloalkyl, C₃₋₁₅ heterocycloalkyl, C₄₋₁₅ aryl, or C₄₋₁₅ heteroaryl or a combination thereof, two or more of Rh, Ri, and Rj optionally being joined to form one or more carbocycles or heterocycles.

The term “alkyl” as used herein may refer to a straight chain or branched, saturated or unsaturated hydrocarbon substituent. Examples of alkyl groups include, but are not limited to, methyl, ethyl, propyl, butyl, pentyl, hexyl, octyl, decyl, isopropyl, sec-butyl, isobutyl, tert-butyl, isopentyl, 2-methylbutyl, vinyl, allyl, 1-butenyl, 2-butenyl, isobutylenyl, 1-pentenyl, and 2-pentenyl.

The term “heteroalkyl” as used herein may refer to a straight chain or branched, saturated or unsaturated hydrocarbon substituent in which at least one carbon is replaced by a heteroatom. Examples include, but are not limited to, methyloxymethyl, ethyloxymethyl, methyloxyethyl, ethyloxyethyl, methylaminomethyl, dimethylaminomethyl, methylaminoethyl, dimethylaminoethyl, methylthiomethyl, ethylthiomethyl, ethylthioethyl, and methylthioethyl.

The term “cycloalkyl” as used herein may refer to a saturated or unsaturated non-aromatic carbocycle substituent, which may consist of one ring or two or more rings fused together. Examples include, but are not limited to, cyclopropyl, cyclobutyl, cyclopentyl, cyclopentenyl, cyclopentadienyl, cyclohexyl, cyclohexenyl, 1,3-cyclohexadienyl, decalinyl, and 1,4-cyclohexadienyl.

The term “heterocycloalkyl” as used herein may refer to a saturated or unsaturated non-aromatic cyclic hydrocarbon substituent, which may consist of one ring or two or more rings fused together, wherein at least one carbon in one of the rings is replaced by a heteroatom. Examples include, but are not limited to, tetrahydrofuranyl, pyrrolidinyl, piperidinyl, 1,4-dioxanyl, decahydroquinolinyl, piperazinyl, oxazolidinyl, and morpholinyl.

The term “heterocycloalkyl” as used herein may refer to a saturated or unsaturated non-aromatic cyclic hydrocarbon substituent, which may consist of one ring or two or more rings fused together, wherein at least one carbon in one of the rings is replaced by a heteroatom. Examples include, but are not limited to, tetrahydrofuranyl, pyrrolidinyl, piperidinyl, 1,4-dioxanyl, decahydroquinolinyl, piperazinyl, oxazolidinyl, and morpholinyl.

The term “alkylaryl” as used herein may refer to an aryl attached to an alkyl, wherein the terms alkyl and aryl are as defined above. Examples include, but are not limited to, benzyl and ethylbenzene radical.

The extension “-ylene” as opposed to “-yl” in for example “alkylene” as opposed to “alkyl” indicates that said for example “alkylene” is a divalent moiety connected to one or two other moieties via two covalent single bonds or one double bond as opposed to being a monovalent group connected to one moiety via one covalent single bond in said for example “alkyl”. The term “alkylene” therefore may refer to a straight chain or branched, saturated or unsaturated hydrocarbon moiety; the term “heteroalkylene” as used herein may refer to a straight chain or branched, saturated or unsaturated hydrocarbon moiety in which at least one carbon is replaced by a heteroatom; the term “arylene” as used herein may refer to a carbocyclic aromatic moiety, which may consist of one ring or two or more rings fused together; the term “heteroarylene” as used herein may refer to a carbocyclic aromatic moiety, which may consist of one ring or two or more rings fused together, wherein at least one carbon in one of the rings is replaced by a heteroatom; the term “cycloalkylene” as used herein may refer to a saturated or unsaturated non-aromatic carbocycle moiety, which may consist of one ring or two or more rings fused together; the term “heterocycloalkylene” as used herein may refer to a saturated or unsaturated non-aromatic cyclic hydrocarbon moiety, which may consist of one ring or two or more rings fused together, wherein at least one carbon in one of the rings is replaced by a heteroatom. Exemplary divalent moieties include those examples given for the monovalent groups hereinabove in which one hydrogen atom is removed.

The prefix “poly” in “polyalkylene”, “polyheteroalkylene”, “polyarylene”, “polyheteroarylene”, polycycloalkylene”, “polyheterocycloalkylene”, and the like, indicates that two or more of such “-ylene” moieties, e.g., alkylene moieties, are joined together to form a branched or unbranched multivalent moiety containing one or more attachment sites for adjacent moieties.

In an embodiment, the alkyl group is unsubstituted or substituted C₁-C₈ alkyl, heteroalkyl, cycloalkyl, heterocycloalkyl, arylalkyl, heteroarylalkyl, alkynyl or alkenyl.

The term “aryl” as used herein may refer to a carbocyclic aromatic substituent, which may consist of one ring or two or more rings fused together. Examples of aryl groups include, but are not limited to, phenyl, naphthyl, and anthracenyl.

The term “heteroaryl” as used herein may refer to a carbocyclic aromatic substituent, which may consist of one ring or two or more rings fused together, wherein at least one carbon in one of the rings is replaced by a heteroatom. Examples of heteroaryl groups include, but are not limited to, pyridinyl, furanyl, pyrrolyl, triazolyl, pyrazolyl, imidazolyl, thiophenyl, indolyl, benzofuranyl, benzimidazolyl, indazolyl, benzotriazolyl, benzisoxazolyl, and quinolinyl.

Certain linkers of the disclosure possess chiral centers or double bonds; the enantiomeric, diastereomeric, and geometric mixtures of two or more isomers, in any composition, as well as the individual isomers are encompassed within the scope of the present disclosure.

In an embodiment, the aryl group is aryl or heteroaryl.

The cytotoxic payload of the present conjugates, E, belongs to the class of camptothecin molecules, which are cytotoxic to the target cells by mechanisms including for example DNA topoisomerase I. Delivery of the cytotoxic payload E to the target cell may thus cause the death of the target cell, such as a cancer cell.

In an embodiment, E is selected from the group consisting of exatecan and a camptothecin analogue comprising a primary or a secondary amino group.

Exatecan comprises an amino group, specifically a primary amino group, as shown e.g. in Formula E1 below.

In an embodiment, E is Exatecan of Formula E1.

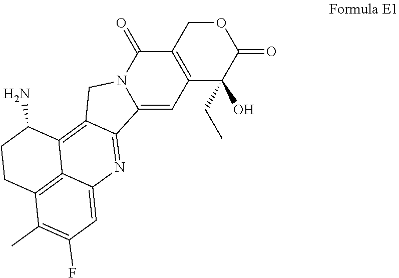 [SMILES: CC[C@@]1(O)C(=O)OCc2c1cc1n(c2=O)Cc2c-1nc1cc(F)c(C)c3c1c2[C@@H](N)CC3]

Camptothecin analogues that do not otherwise comprise an amino group may be substituted by a primary and/or secondary amino group. In other words, the camptothecin analogue may be a camptothecin or a camptothecin analogue substituted by a primary and/or secondary amino group.

In an embodiment, E is 2-hydroxyacetoyl exatecan (DXd) of Formula E2.

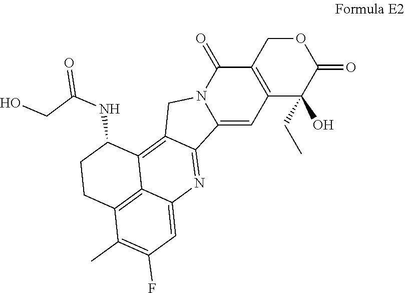 [SMILES: CC[C@@]1(O)C(=O)OCc2c1cc1n(c2=O)Cc2c-1nc1cc(F)c(C)c3c1c2[C@@H](NC(=O)CO)CC3]

In an embodiment, E is NH₂—CH₂—O—CH₂—C(O)-exatecan (NH₂—CH₂-DXd) of Formula E3.

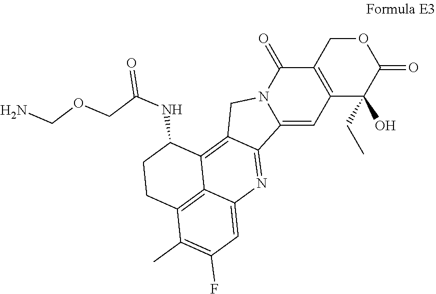 [SMILES: CC[C@@]1(O)C(=O)OCc2c1cc1n(c2=O)Cc2c-1nc1cc(F)c(C)c3c1c2[C@@H](NC(=O)COCN)CC3]

In an embodiment, E is a camptothecin analogue.

In an embodiment, E is selected from the group consisting of camptothecin, CPT-11, lurtotecan, atiratecan, SN-38, topotecan, irinotecan and belotecan.

In an embodiment, the camptothecin analogue comprises (i.e. is substituted by) a primary or secondary amino group, through which the camptothecin analogue is bonded so as to form an amide group together with the carbonyl group to which it is bonded.

In an embodiment, the camptothecin analogue is 9-aminocamptothecin.

In an embodiment, E is 9-aminocamptothecin of Formula E4.

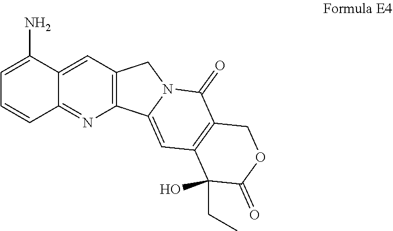 [SMILES: CC[C@@]1(O)C(=O)OCc2c1cc1n(c2=O)Cc2cc3c(N)cccc3nc2-1]

In an embodiment, R₁ is a spacer group.

The spacer group connects the linker of the present disclosure to the targeting unit. In an embodiment, the targeting unit is an antibody and the spacer group connects the linker to an amino acid side chain of the antibody with a covalent bond. In an embodiment, the covalent bond is an amide bond. In an embodiment, the covalent bond is a thioether bond. In an embodiment, the amino acid side chain of the antibody is the side chain of cysteine. In an embodiment, the amino acid side chain of the antibody is the side chain of lysine.

As used herein, “amino acid side chain” refers the monovalent hydrogen or non-hydrogen substituent bonded to the α-carbon of an α-amino acid, including α-amino acid and non-α-amino acids. Exemplary amino acid side chains include, but are not limited to, the α-carbon substituent of glycine, alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, and citrulline.

An amino acid according to the present disclosure may be in L- or D-configuration; in free amino acid or amino acid residue form; and any combination thereof.

In the context of the linker-payload conjugate, R₁ may be a spacer group selected from the group consisting of a maleimidoacetyl, maleimidoacetyl-β-alanyl, and a C₂-C₈ acyl group comprising a bioorthogonal conjugation group.

In the context of the targeting unit-linker-payload conjugate, R₁ may be a spacer group selected from the group consisting of

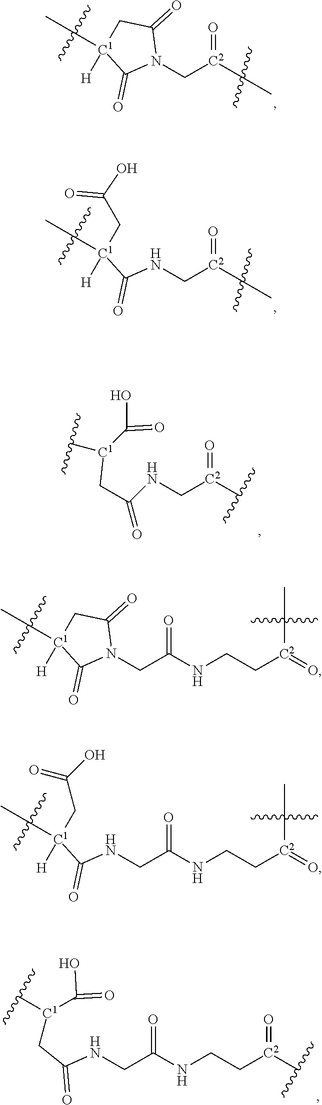

and a C₂-C₈ acyl group comprising a radical of a bioorthogonal conjugation group, wherein C¹ is covalently attached to the targeting unit, optionally covalently attached to to a sulphur of the targeting unit, and C₂ is covalently attached to the nitrogen of NH.

In an embodiment, the C¹ atom of the spacer group R₁ is in R configuration. In an embodiment, the C¹ atom of the spacer group R₁ is in S configuration. In an embodiment, the C¹ atom of the spacer group R₁ is a mixture of R and S configurations.

In an embodiment, the spacer group comprises an acyl group conjugated to the rest of the linker with an amide bond.

In an embodiment, the spacer group comprises an alkyl group conjugated to the rest of the linker with an amine bond.

In an embodiment, the spacer group comprises a bioorthogonal linking group.

In an embodiment, the spacer group comprises a bioorthogonal linking group selected from the group consisting of azide, alkyne, triazole, maleimide, thiol, amine, carboxylic acid, amide, alkene, ether, thioether, —CHO, ketone, hydroxylamine, hemiacetal, acetal, phosphine, tetrazine, cyclooctene, nitrone, isoxazoline, nitrile oxide, norbornene, oxanorbornadiene, tetrazole, pyrazoline and quadricyclane.

In an embodiment, the bioorthogonal linking group is a 1,3-triazole.

In an embodiment, the bioorthogonal linking group is an alkyne selected from the group of aliphatic alkyne such as a propargyl group or a cycloalkyne such as DBCO, DIBO, cyclononyne, cyclooctyne, and the like.

In an embodiment, the bioorthogonal linking group is an azidyl.

In the present specification and its embodiments, “maleimidyl” may refer to both an intact maleimidyl and a hydrolyzed maleimidyl connected to the targeting unit with a thioether bond. In an embodiment, the spacer group comprises a maleimidyl group or a group derived from a maleimidyl group.

In an embodiment, the spacer group comprises a hydrolyzed maleimidyl group.

In an embodiment, the spacer group comprises a maleimidyl group connected to the targeting unit with a thioether bond.

In an embodiment, the maleimidyl group or the group derived from the maleimidyl group connected to the targeting unit with a thioether bond is hydrolyzed after the conjugation. In an embodiment, the structure of the spacer group comprising the maleimidyl group has the ability to self-hydrolyze, in other words self-stabilize, in neutral pH or near-neutral pH. The hydrolysis and the stabilization of the maleimide with a thioether bond promotes both efficacy of the conjugate (when more cytotoxic payload reaches the target cell) and tolerability and safety of the conjugate (when less cytotoxic payload is released to non-target tissues and non-target cells).

For example, the group shown in the context of the targeting unit-linker-payload conjugate as

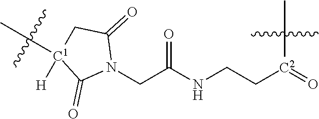

may hydrolyse into either one of the following structures:

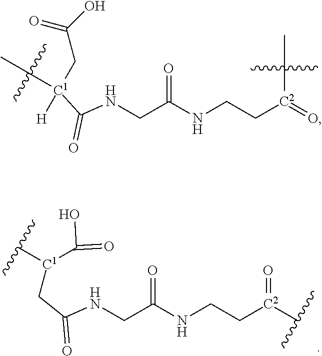

In an embodiment, the spacer group s maleimidopropionate or maleimiodohexanoate group.

In an embodiment of the linker-payload conjugate, R₁ is selected from the group consisting of maleimidoacetyl-β-alanyl and maleimidoacetyl. The maleimidoacetyl structure was shown in the present examples to efficiently facilitate the self-hydrolysis and self-stabilization of the maleimide group after conjugation.

In an embodiment, R₁ is maleimidoacetyl-β-alanyl.

In an embodiment, R₁ is maleimidoacetyl.

In an embodiment of the targeting unit-linker-payload conjugate, R₁ is a spacer group selected from the group consisting of

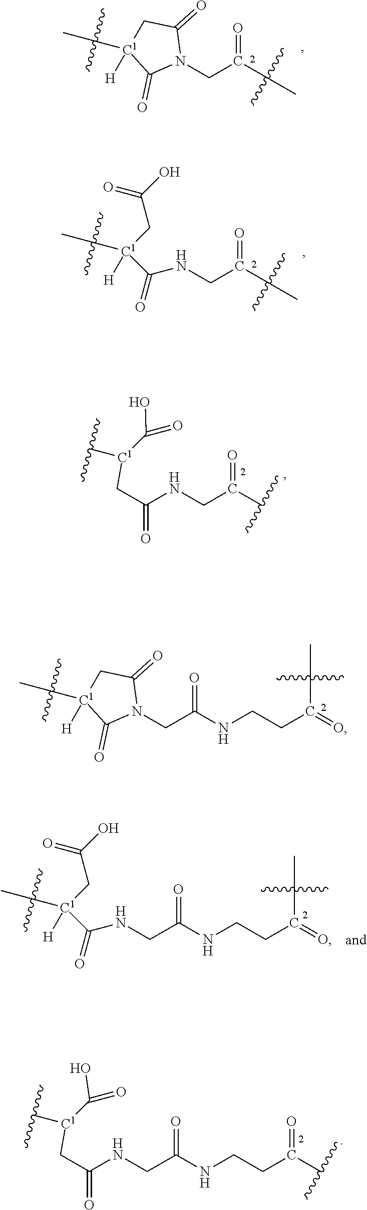

The group derived from the maleimidoacetyl structure was shown in the present examples to efficiently facilitate the self-hydrolysis and self-stabilization of the maleimide group after conjugation.

In an embodiment, R₁ is selected from the group consisting of

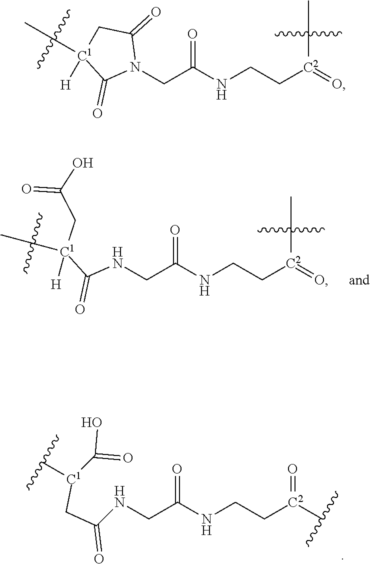

In an embodiment, R₁ is selected from the group consisting of

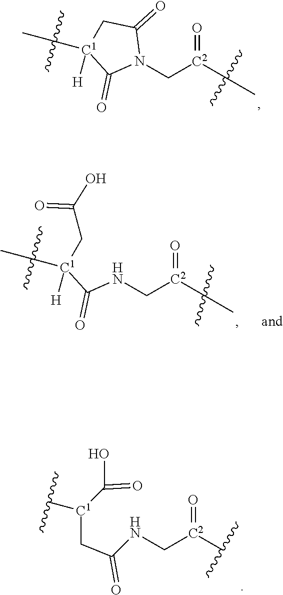

In the above embodiments, C¹ may be covalently attached to a sulphur of the targeting unit (i.e. via a thioether bond).

In an embodiment, R₃ is a spacer group or absent, wherein the spacer group is selected from the group consisting of a prodrug group and —NH—CH₂—O—CH₂—C(O)—.

The group —NH—CH₂—O—CH₂—C(O)— should be understood as referring to —NH—CH₂—O—CH₂—C(═O)—.

In an embodiment, the prodrug group comprises a primary or a secondary amino group, to which the linker is conjugated with an amide bond.

In an embodiment, the prodrug group is selected from the group consisting of para-aminobenzyloxycarbonyl (PABC), orto-aminobenzyloxycarbonyl, —OCH₂NH—, an amino acid residue, and a peptide. In an embodiment, the prodrug group is selected from the group consisting of a radical of para-aminobenzyloxycarbonyl (PABC), a radical of orto-aminobenzyloxycarbonyl, —OCH₂NH—, an amino acid residue, and a radical of a peptide.

In an embodiment, R₁ is maleimidoacetyl-β-alanyl; R₂ is β-D-glucose; R₃ is —NH—CH₂—O—CH₂—C(O)—; and E is exatecan.

In an embodiment, R₁ is maleimidoacetyl-β-alanyl; R₂ is β-D-glucuronic acid; R₃ is —NH—CH₂—O—CH₂—C(O)—; and E is exatecan.

In an embodiment, R₁ is maleimidoacetyl-β-alanyl; R₂ is β-D-glucose; R₃ is absent; and E is exatecan.

In an embodiment, R₁ is maleimidoacetyl-β-alanyl; R₂ is β-D-glucuronic acid; R₃ is absent; and E is exatecan.

In an embodiment, R₁ is maleimidoacetyl-β-alanyl; R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is

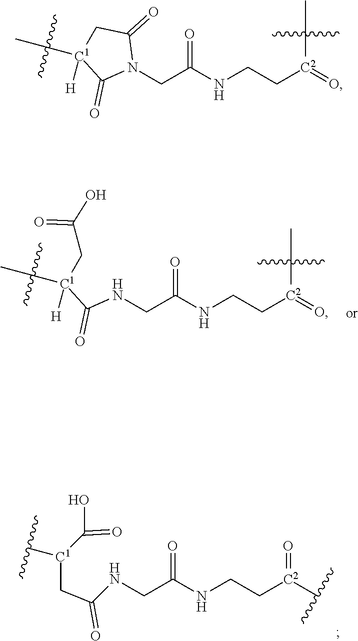

R₂ is β-D-glucuronic acid; R₃ is —NH—CH₂—O—CH₂—C(O)—; and E is exatecan.

In an embodiment, R₁

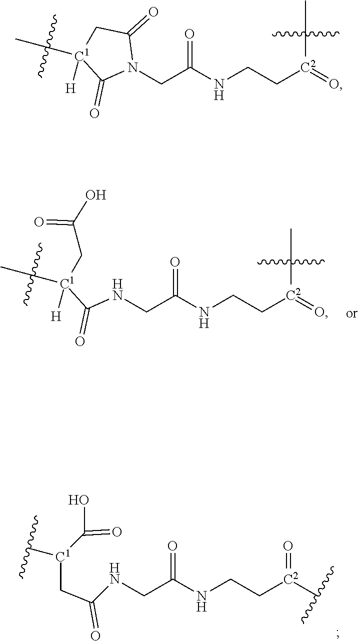

R₂ is β-D-glucose; R₃ is absent; and E is exatecan.

In an embodiment, R₁ is

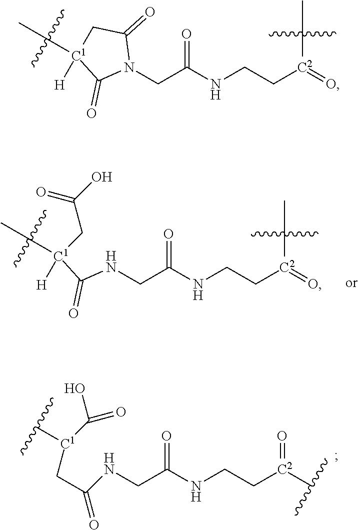

R₂ is β-D-glucuronic acid; R₃ is absent; and E is exatecan.

In an embodiment, R₁ is

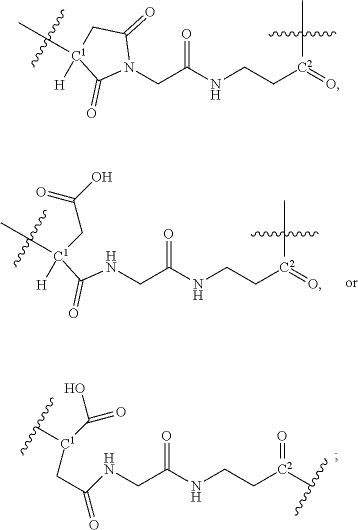

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is maleimidoacetyl-β-alanyl; R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is maleimidoacetyl-β-alanyl; R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is maleimidoacetyl-β-alanyl; R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is

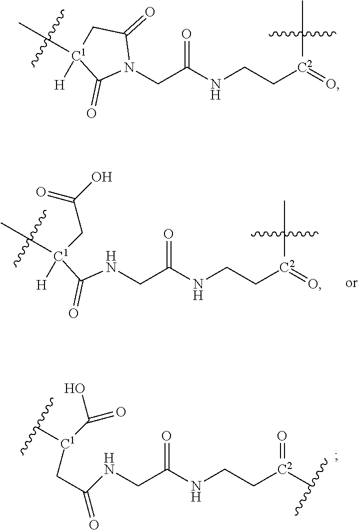

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is

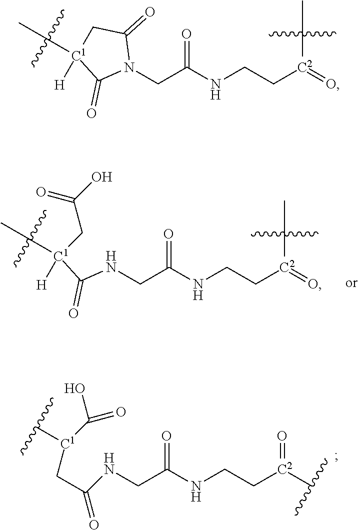

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, R₁ is

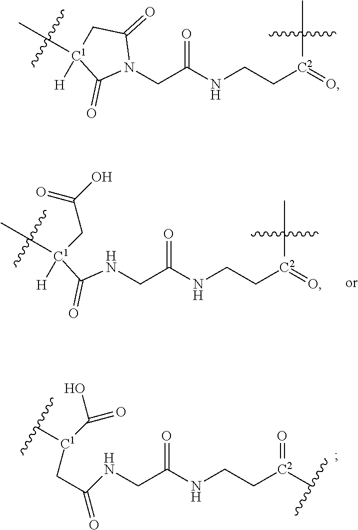

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, the targeting unit T is a molecule that is capable of specifically binding to a target molecule. In the context of the present disclosure, the specific binding has the meaning that the targeting molecule has a reasonably higher binding affinity to its target than to unrelated molecules. An example of the specific binding is the binding of an antibody to its target epitope.

In an embodiment, the targeting unit T is a molecule that is capable of specifically binding to a target molecule on a surface of a target cell.

In an embodiment, the targeting unit T is small-molecule weight ligand, a lectin, a peptide, an aptamer, or an antibody.

In an embodiment, the targeting unit T is an antibody.

The antibody may, in principle, be any antibody or its binding fragment, for instance an IgG, an scFv, a single domain antibody, an Fv, a VHH antibody, a diabody, a tandem diabody, a Fab, a Fab′, a F(ab′)2, a Db, a dAb-Fc, a taFv, a scDb, a dAb2, a DVD-Ig, a Bs(scFv)4-IgG, a taFv-Fc, a scFv-Fc-scFv, a Db-Fc, a scDb-Fc, a scDb-CH3, or a dAb-Fc-dAb.

In an embodiment, the antibody is a human antibody or a humanized antibody. In this context, the term “human antibody”, as it is commonly used in the art, is to be understood as meaning antibodies having variable regions in which both the framework and complementary determining regions (CDRs) are derived from sequences of human origin. In this context, the term “humanized antibody”, as it is commonly used in the art, is to be understood as meaning antibodies wherein residues from a CDR of an antibody of human origin are replaced by residues from a CDR of a nonhuman species (such as mouse, rat or rabbit) having the desired specificity, affinity and capacity.

In an embodiment, the antibody is capable of binding a cell surface antigen.

In an embodiment, the cell surface antigen is a tumor antigen and/or a cancer antigen.

In an embodiment, the antibody is selected from the group consisting of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan.

In an embodiment, the antibody is capable of specifically binding a target molecule selected from the group consisting of CD2, CD3, CD4, CD5, CD6, CD11, CD8, CD11a, CD19, CD20, CD22, CD25, CD26, CD30, CD33, CD34, CD37, CD38, CD40, CD44, CD46, CD52, CD56, CD79, CD105, CD138, epidermal growth factor receptor 1 (EGFR), epidermal growth factor receptor 2 (HER2/neu), HER3 or HER4 receptor, LFA-1, Mac1, p150.95, VLA-4, ICAM-1, VCAM, EpCAM, alpha4/beta7 integrin, alpha v/beta3 integrin including either alpha or beta subunits thereof (e.g. anti-CD11a, anti-CD18 or anti-CD11b antibodies), tissue factor (TF), tumor necrosis factor alpha (TNF-α), human vascular endothelial growth factor (VEGF), glycoprotein IIb/IIIa, TGF-beta, alpha interferon (alpha-IFN), IL-8, IL-2 receptor, IgE, respiratory syncytial virus (RSV), HIV-1 envelope glycoprotein gp120, cancer-associated high-mannose type N-glycans, blood group antigen Apo2, death receptor, flk2/flt3 receptor, obesity (OB) receptor, mpl receptor, CTLA-4, transferrin receptor, Lewis y, GD3 and protein C.

In an embodiment, the antibody is selected from the group consisting of abagovomab, actoxumab, adecatumumab, afutuzumab, altumomab, amatuximab, anifrolumab, apolizumab, atinumab, atlizumab, atorolimumab, bapineuzumab, basiliximab, bavituximab, belimumab, benralizumab, bertilimumab, besilesomab, bezlotoxumab, bimagrumab, bivatuzumab, blinatumomab, blosozumab, brentuximab, briakinumab, brodalumab, canakinumab, cantuzumab, caplacizumab, capromab, carlumab, catumaxomab, CC49, cedelizumab, cixutumumab, clazakizumab, clenoliximab, clivatuzumab, conatumumab, concizumab, crenezumab, CR6261, dacetuzumab, dalotuzumab, daratumumab, demcizumab, denosumab, detumomab, drozitumab, duligotumab, dupilumab, dusigitumab, ecromeximab, eculizumab, edobacomab, edrecolomab, eldelumab, elotuzumab, elsilimomab, enavatuzumab, enlimomab, enokizumab, enoticumab, ensituximab, epitumomab, epratuzumab, ertumaxomab, etaracizumab, etrolizumab, evolocumab, exbivirumab, fanolesomab, faralimomab, farletuzumab, fasinumab, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gevokizumab, girentuximab, glembatumumab, golimumab, gomiliximab, guselkumab, ibalizumab, icrucumab, imciromab, imgatuzumab, inclacumab, indatuximab, intetumumab, inolimomab, inotuzumab, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, ligelizumab, lintuzumab, lirilumab, lodelcizumab, lorvotuzumab, lucatumumab, lumiliximab, mapatumumab, margetuximab, maslimomab, mavrilimumab, matuzumab, mepolizumab, metelimumab, milatuzumab, minretumomab, mitumomab, mogamulizumab, morolimumab, motavizumab, moxetumomab, muromonab, namilumab, narnatumab, natalizumab, nebacumab, necitumumab, nerelimomab, nesvacumab, nimotuzumab, nivolumab, obinutuzumab, ocaratuzumab, ocrelizumab, odulimomab, ofatumumab, olaratumab, olokizumab, onartuzumab, oregovomab, orticumab, otelixizumab, oxelumab, ozanezumab, ozoralizumab, pagibaximab, panobacumab, parsatuzumab, pascolizumab, pateclizumab, patritumab, pemtumomab, perakizumab, pertuzumab, pidilizumab, pinatuzumab, pintumomab, placulumab, polatuzumab, ponezumab, priliximab, pritoxaximab, pritumumab, quilizumab, racotumomab, radretumab, rafivirumab, ramucirumab, raxibacumab, regavirumab, reslizumab, rilotumumab, robatumumab, roledumab, romosozumab, rontalizumab, rovelizumab, ruplizumab, samalizumab, sarilumab, satumomab, secukinumab, seribantumab, setoxaximab, sevirumab, sibrotuzumab, sifalimumab, siltuximab, simtuzumab, siplizumab, sirukumab, solanezumab, solitomab, sonepcizumab, sontuzumab, stamulumab, suvizumab, tabalumab, tacatuzumab, talizumab, tanezumab, taplitumomab, tefibazumab, tenatumomab, teneliximab, teplizumab, teprotumumab, TGN1412, ticilimumab, tildrakizumab, tiga-tuzumab, tocilizumab, toralizumab, tovetumab, tralokinumab, TRBS07, tregalizumab, tremelimumab, tucotuzumab, tuvirumab, ublituximab, urelumab, urtoxazumab, ustekinumab, vantictumab, vapaliximab, vatelizumab, vedolizumab, veltuzumab, vepalimomab, vesencumab, visilizumab, volociximab, vorsetuzumab, votumumab, zalutumumab, zanolimumab, zatuximab, ziralimumab, 2G12 (anti-HIV-1 envelope glycoprotein gp120), and zolimomab.

In an embodiment, the antibody is selected from the group consisting of an anti-EGFR1 antibody, an epidermal growth factor receptor 2 (HER2/neu) antibody, an anti-CD22 antibody, an anti-CD30 antibody, an anti-CD33 antibody, and an anti-CD20 antibody.

In an embodiment, the antibody is an anti-EGFR antibody.

In an embodiment, an anti-EGFRI antibody is cetuximab, imgatuzumab, matuzumab, nimotuzumab, necitumumab, panitumumab, or zalutumumab.

In an embodiment, the antibody is an epidermal growth factor receptor 2 (HER2/neu) antibody.

In an embodiment, an anti-HER2 antibody is margetuximab, pertuzumab, trastuzumab, ertumaxomab, or 520C9XH22.

In an embodiment, the antibody is an anti-CD22 antibody.

In an embodiment, an anti-CD22 antibody is bectumomab, moxetumomab, epratuzumab, inotuzumab, or pinatuzumab.

In an embodiment, the antibody is an anti-CD30 antibody.

In an embodiment, an anti-CD30 antibody is brentuximab vedotin (or the antibody portion of the brentuximab vedotin) or iratumumab.

In an embodiment, the antibody is an anti-CD33 antibody.

In an embodiment, an anti-CD33 antibody is gemtuzumab, SGN-CD33A or lintuzumab.

Targeting unit-linker-payload conjugates can be prepared using cross-linking reagents. For example, a cysteine, thiol or an amine, e.g. N-terminus or an amino acid side chain, such as lysine of the antibody, can form a bond with a functional group of a cross-linking reagent.

The linkers according to the present disclosure can be prepared by standard methods known to a person skilled in the art. For example, the central amino acid and peptide groups of the linker can be prepared by standard peptide chemistry and automated peptide chemistry, and ordered from a commercial manufacturer of synthetic peptides; and the saccharide, sulfate, phosphate, phosphodiester and phosphonate group R₂ can be added to the amino acid and peptide groups during or after their synthesis from commercially available protected building blocks. Further, the acyl group R₁ and/or the spacer group R₃ can be added to the amino acid and peptide groups by standard chemistry forming amide bonds to the amino acid and peptide groups.

General methods to prepare the targeting unit—linker-payload conjugates, i.e. addition of the payload E and the targeting unit T, are known for the skilled artisan, and for example, described in WO/2016/001485, WO/2014/096551 and WO/2014/177771.

The targeting unit-linker-payload conjugates and linker-payload conjugates can be characterized and selected for their physical/chemical properties and/or biological activities by various assays known in the art.

For example, a conjugate can be tested for its antigen binding activity by known methods such as ELISA, FACS, Biacore or Western blot.

Transgenic animals and cell lines are particularly useful in screening conjugates that have potential as prophylactic or therapeutic treatments of cancer of tumor-associated antigens and cell surface receptors. Screening for a useful conjugate may involve administering a candidate conjugate over a range of doses to the transgenic animal, and assaying at various time points for the effect(s) of the conjugate on the disease or disorder being evaluated. Alternatively, or additionally, the drug can be administered prior to or simultaneously with exposure to an inducer of the disease, if applicable. The candidate conjugate may be screened serially and individually, or in parallel under medium or high-throughput screening format.

A method for preparing the targeting unit-linker-payload conjugate according to one or more embodiments is disclosed, comprising conjugating the linker-payload according to one or more embodiments to a targeting unit, optionally via a spacer group.

Many ways of conjugating payload molecules to targeting units, for example, antibodies are known, and in principle any way that is suitable for conjugating a payload to a targeting unit may be used. The linker-payload according to one or more embodiments may be conjugated to the targeting unit such as an antibody directly or indirectly, for instance via a spacer group. In an embodiment, the linker-payload according to one or more embodiments and comprising a maleimide is conjugated to the antibody by reducing hinge region cysteines with a reducing agent and contacting the reduced antibody with the linker-payload to form thioether bond.

In this context, the antibody may in principle be any antibody, and in particular any antibody described in this specification.

In an embodiment, the targeting unit-linker-payload conjugate comprises or is a targeting unit-linker-payload conjugate according to Formula III, wherein

T is an antibody; n is in the range of 1 and about 20; R₁ is

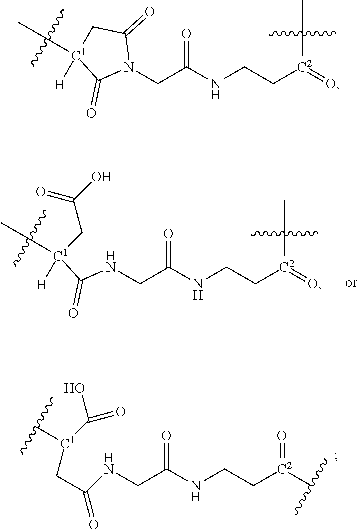

R₂ is β-D-glucose; R₃ is —NH—CH₂—O—CH₂—C(O)—; and E is exatecan;

T is an antibody; n is in the range of 1 and about 20; R₁ is

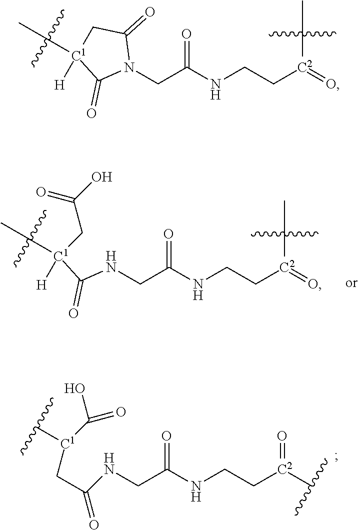

R₂ is β-D-glucuronic acid; R₃ is —NH—CH₂—O—CH₂—C(O)—; and E is exatecan;

T is an antibody; n is in the range of 1 and about 20; R₁ is

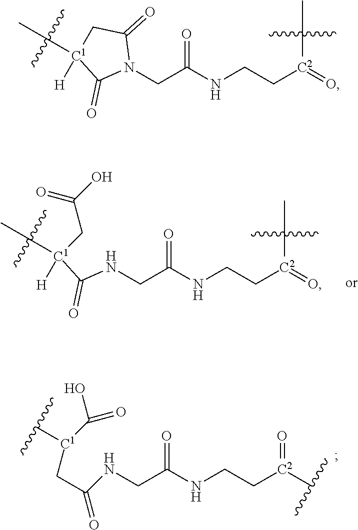

R₂ is β-D-glucose; R₃ is absent; and E is exatecan;

T is an antibody; n is in the range of 1 and about 20; R₁ is

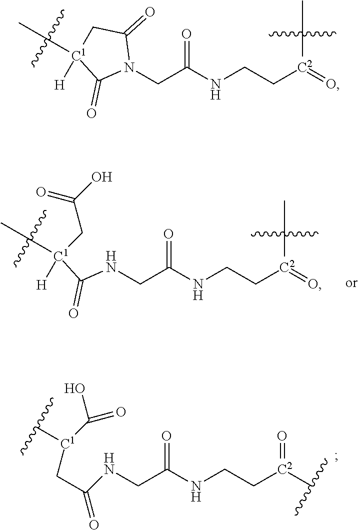

R₂ is β-D-glucuronic acid; R₃ is absent; and E is exatecan;

T is an antibody; n is about 8; R₁ is

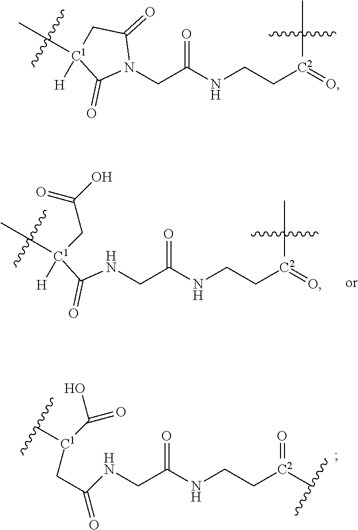

R₂ is β-D-glucose; R₃ is —NH—CH₂—O—CH₂—C(O)—; and E is exatecan;

T is an antibody; n is about 8; R₁ is

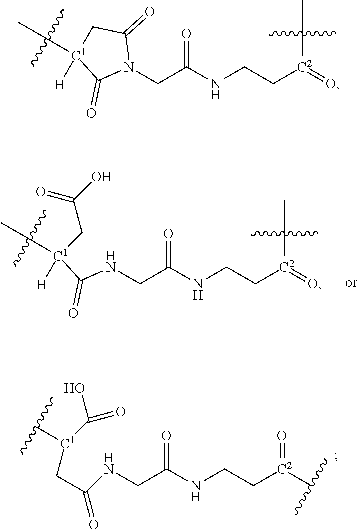

R₂ is β-D-glucuronic acid; R₃ is —NH—CH₂—O—CH₂—C(O)—; and E is exatecan;

T is an antibody; n is about 8; R₁ is

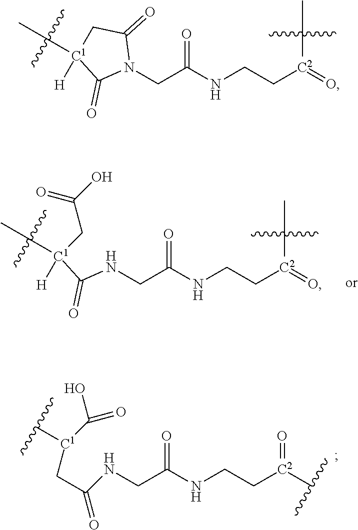

R₂ is β-D-glucose; R₃ is absent; and E is exatecan;

T is an antibody; n is about 8; R₁ is

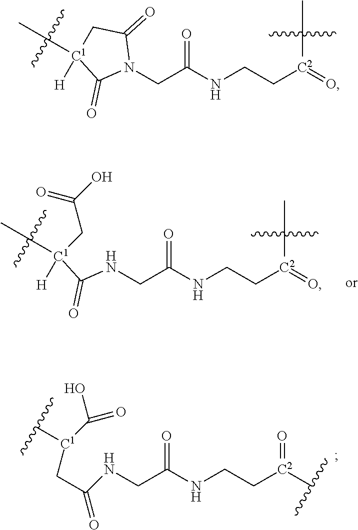

R₂ is β-D-glucuronic acid; R₃ is absent; and E is exatecan;

T is an antibody; n is in the range of 1 and about 20; R₁ is

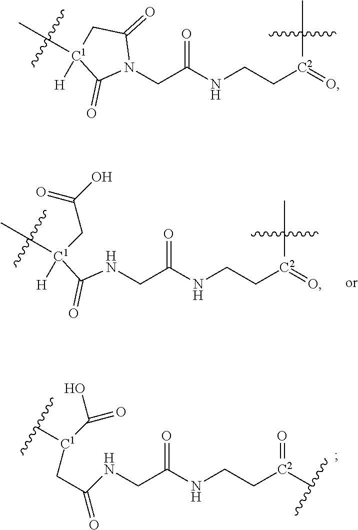

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is in the range of 1 and about 20; R₁ is

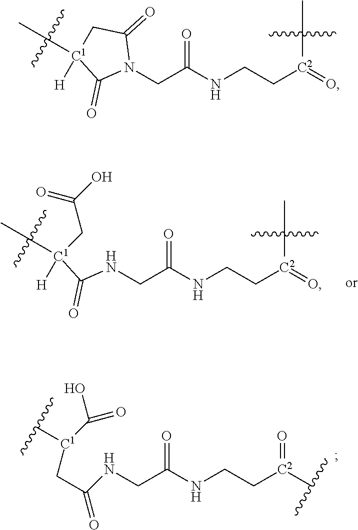

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is in the range of 1 and about 20; R₁ is

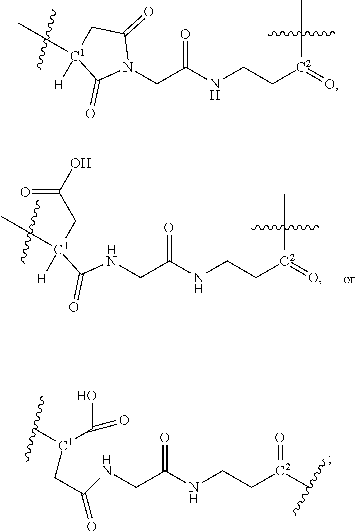

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is in the range of 1 and about 20; R₁ is

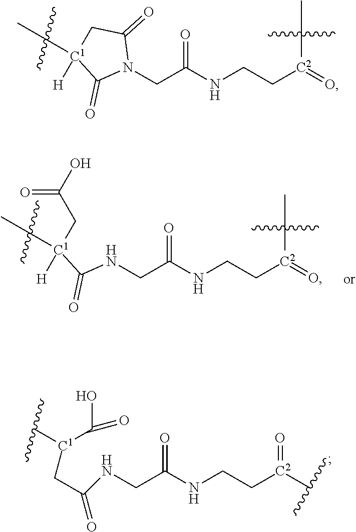

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

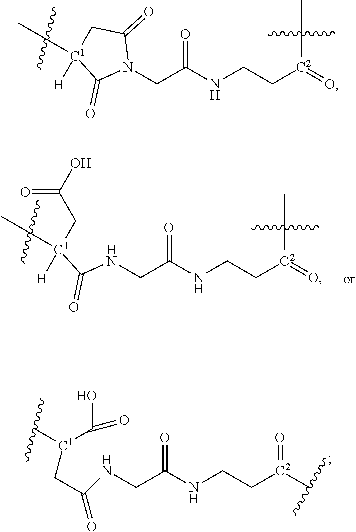

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

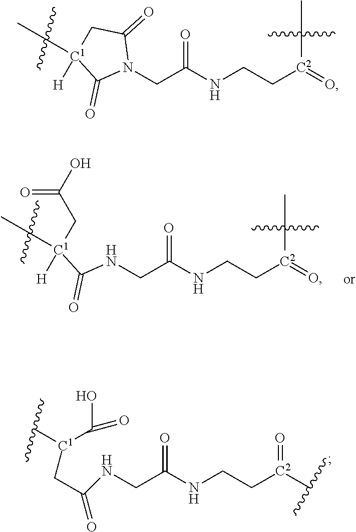

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

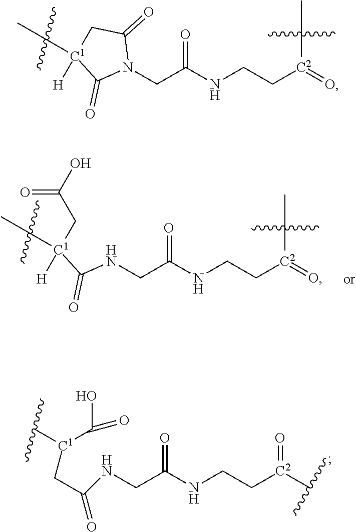

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

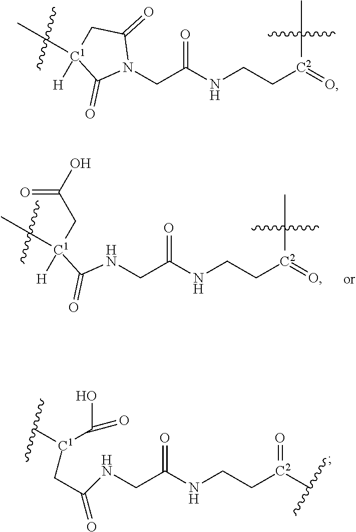

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, the targeting unit-linker-payload conjugate comprises or is a targeting unit-linker-payload conjugate according to Formula IIIB, wherein

T is an antibody; n is in the range of 1 and about 20; R₁ is

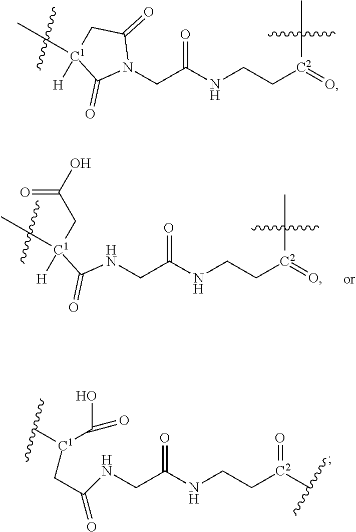

R₂ is β-D-glucose; R₃ is —NH—CH₂—O—CH₂—C(O)—; and E is exatecan;

T is an antibody; n is in the range of 1 and about 20; R₁ is

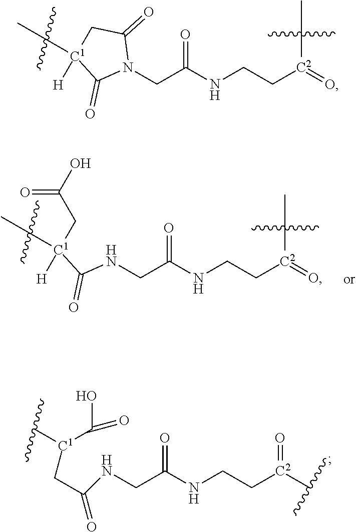

R₂ is β-D-glucuronic acid; R₃ is —NH—CH₂—O—CH₂—C(O)—; and E is exatecan;

T is an antibody; n is in the range of 1 and about 20; R₁ is

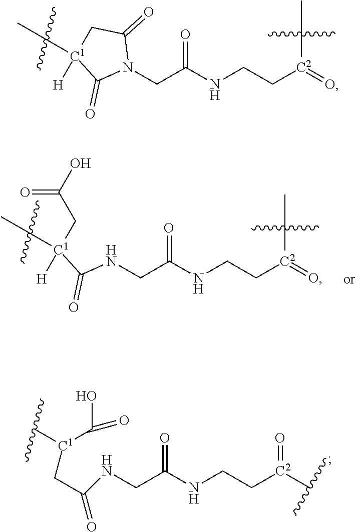

R₂ is β-D-glucose; R₃ is absent; and E is exatecan;

T is an antibody; n is in the range of 1 and about 20; R₁ is

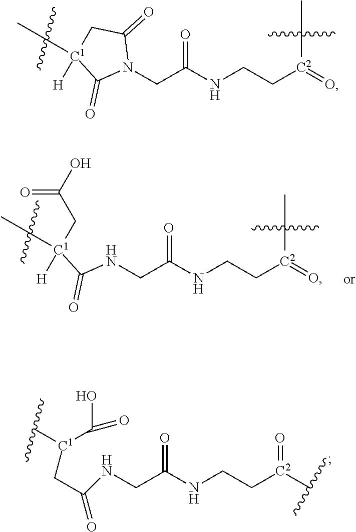

R₂ is β-D-glucuronic acid; R₃ is absent; and E is exatecan;

T is an antibody; n is about 8; R₁ is

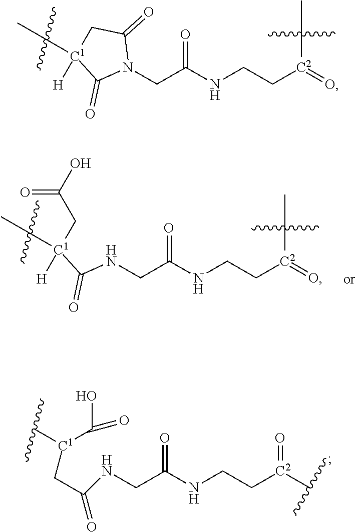

R₂ is β-D-glucose; R₃ is —NH—CH₂—O—CH₂—C(O)—; and E is exatecan;

T is an antibody; n is about 8; R₁ is

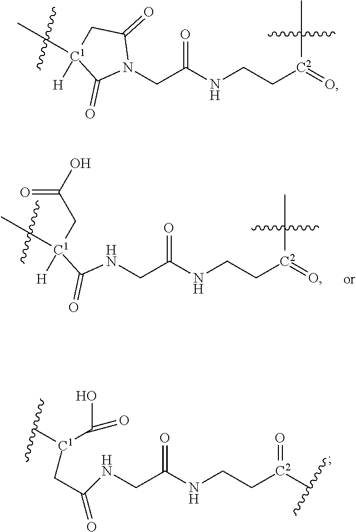

R₂ is β-D-glucuronic acid; R₃ is —NH—CH₂—O—CH₂—C(O)—; and E is exatecan;

T is an antibody; n is about 8; R₁ is

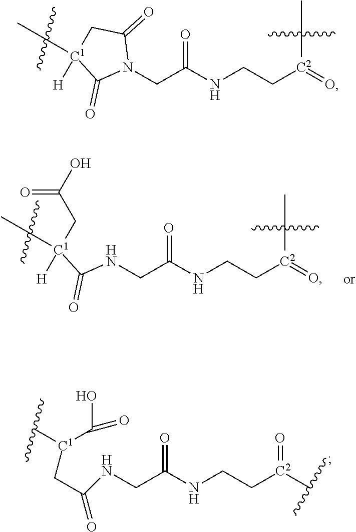

R₂ is β-D-glucose; R₃ is absent; and E is exatecan;

T is an antibody; n is about 8; R₁ is

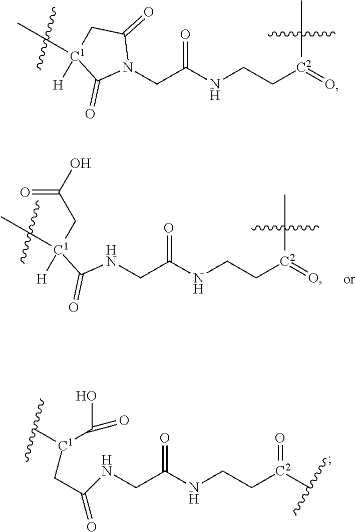

R₂ is β-D-glucuronic acid; R₃ is absent; and E is exatecan;

T is an antibody; n is in the range of 1 and about 20; R₁ is

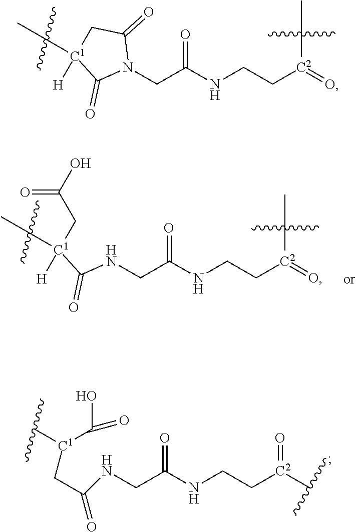

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is in the range of 1 and about 20; R₁ is

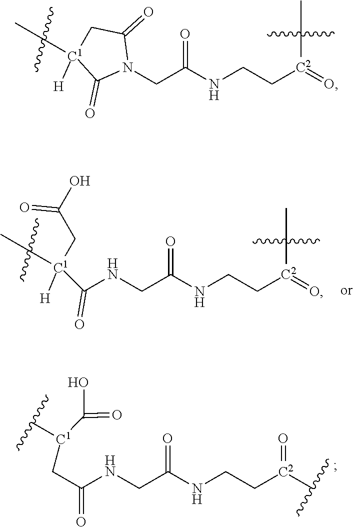

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody: n is in the range of 1 and about 20: R₁ is

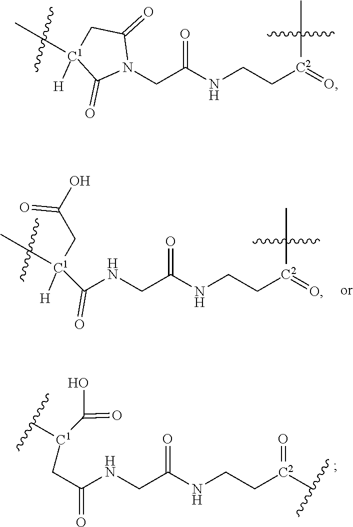

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is in the range of 1 and about 20; R₁ is

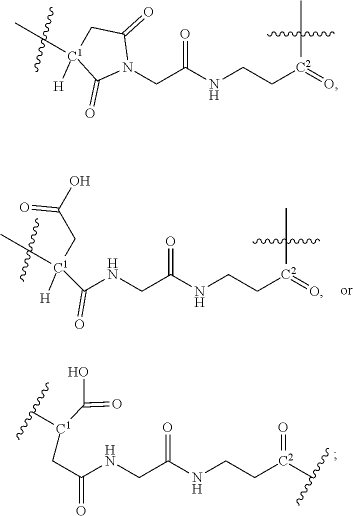

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

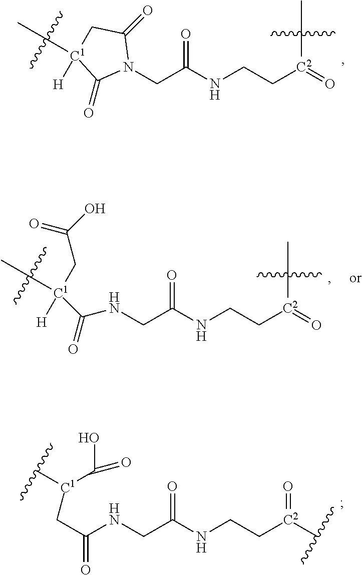

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

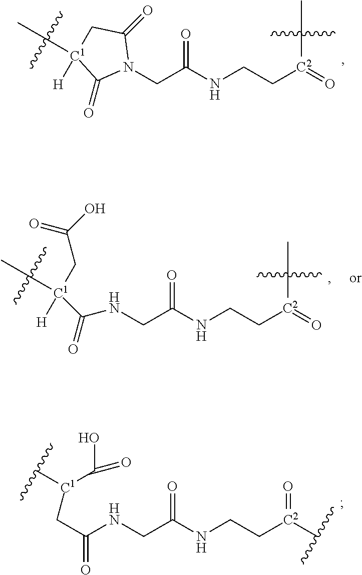

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody; n is about 8; R₁ is

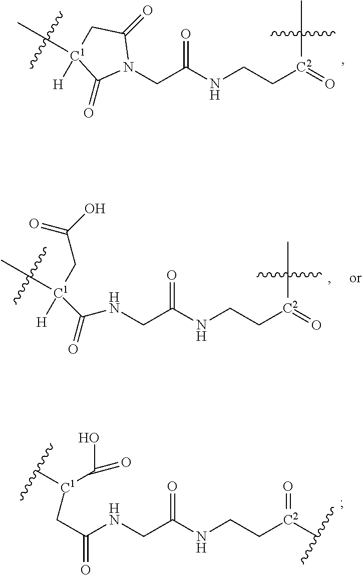

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is an antibody: n is about 8: R₁ is

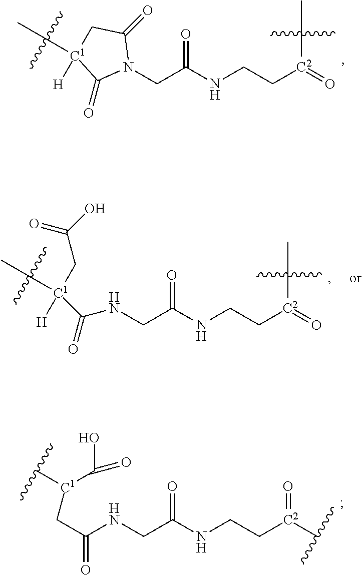

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

In an embodiment, n is in the range of 1 to about 20, or in the range of 1 to about 15, or in the range of 1 to about 10, or in the range of 2 to 10, or in the range of 2 to 6, or in the range of 2 to 5, or in the range of 2 to 4; or n is 1, 2, 3, 4, 5, 6, 7, 8, 9, 10, 11, 12, 13, 14, 15, 16, 17, 18, 19, or 20.

In an embodiment, n is in the range of 3 to about 20, or in the range of 3 to about 15, or in the range of 3 to about 10, or in the range of 3 to about 9, or in the range of 3 to about 8, or in the range of 3 to about 7, or in the range of 3 to about 6, or in the range of 3 to 5, or in the range of 3 to 4.

In an embodiment, n is in the range of 4 to about 20, or in the range of 4 to about 15, or in the range of 4 to about 10, or in the range of 4 to about 9, or in the range of 4 to about 8, or in the range of 4 to about 7, or in the range of 4 to about 6, or in the range of 4 to 5.

In an embodiment, n is 5.

In an embodiment, n is 6.

In an embodiment, n is 7.

In an embodiment, n is 8.

In an embodiment, n is 9.

In an embodiment, n, or drug-to-antibody (DAR) ratio, of a targeting unit-linker-payload conjugate may be determined using a MALDI-TOF MS.

In an embodiment, n, or drug-to-antibody ratio, of a targeting unit-linker-payload conjugate may be determined using an ESI-MS.

Exemplary methods to determine n, or drug-to-antibody ratio, is described in Chen J, Yin S, Wu Y, Ouyang J. Development of a native nanoelectrospray mass spectrometry method for determination of the drug-to-antibody ratio of antibody-drug conjugates. Anal Chem. 2013 Feb. 5;85(3):1699-1704. doi:10.1021/ac302959p.

In an embodiment, the antibody is selected from the group of an anti-EGFR1 antibody, cetuximab, imgatuzumab, matuzumab, nimotuzumab, necitumumab, panitumumab, zalutumumab, an epidermal growth factor receptor 2 (HER2/neu) antibody, margetuximab, pertuzumab, trastuzumab, ertumaxomab, 520C9XH22, an anti-CD22 antibody, bectumomab, moxetumomab, epratuzumab, inotuzumab, pinatuzumab, an anti-CD30 antibody, brentuximab, iratumumab, an anti-CD33 antibody, gemtuzumab, SGN-CD33A, lintuzumab, tositumomab, alemtuzumab, an anti-CD20 antibody, rituximab, epitumomab, ublituximab, obinutuzumab, ocaratuzumab, ocrelizumab, veltuzumab, ofatumumab, nofetumomab and ibritumomab or from the group consisting of an anti-EGFR1 antibody, an epidermal growth factor receptor 2 (HER2/neu) antibody, an anti-CD22 antibody, an anti-CD30 antibody, an anti-CD33 antibody, and an anti-CD20 antibody.

In an embodiment, the targeting unit-linker-payload conjugate comprises or is a targeting unit-linker-payload conjugate according to Formula III or Formula IIIB, wherein

T is trastuzumab; n is about 8; R₁ is

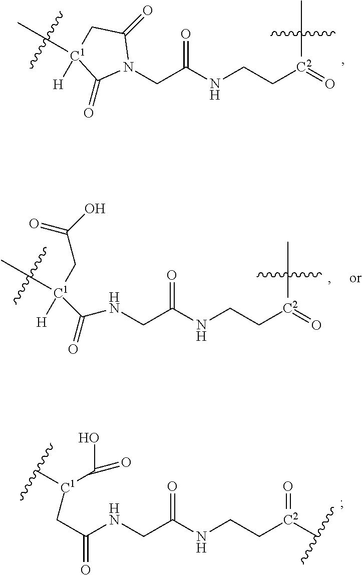

R₂ is β-D-glucose; R₃ is —NH—CH₂—O—CH₂—C(O)—; and E is exatecan;

T is trastuzumab; n is about 8; R₁ is

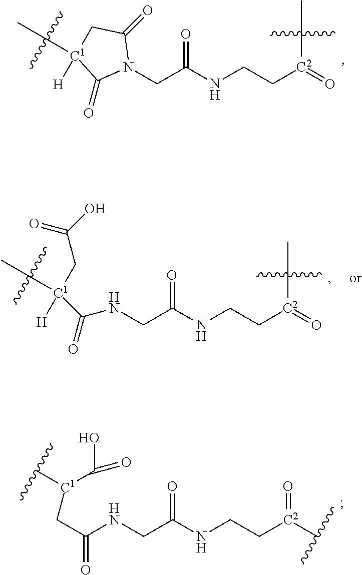

R₂ is β-D-glucuronic acid; R₃ is —NH—CH₂—O—CH₂—C(O)—; and E is exatecan;

T is trastuzumab; n is about 8; R₁ is

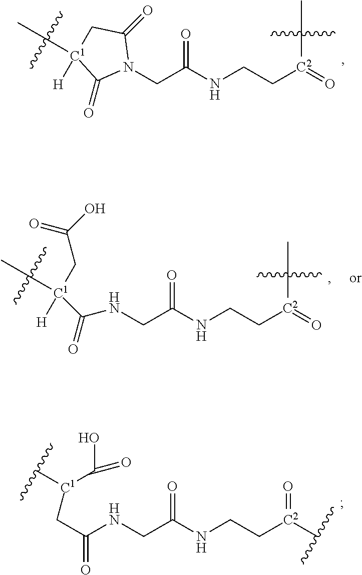

R₂ is β-D-glucose; R₃ is absent; and E is exatecan;

T is trastuzumab; n is about 8; R₁ is

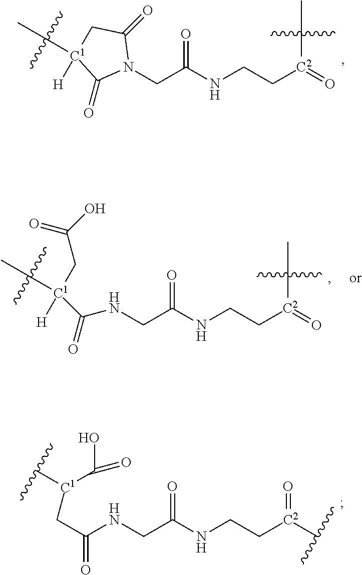

R₂ is β-D-glucuronic acid; R₃ is absent; and E is exatecan;

T is flanvotumab; n is about 8; R₁ is

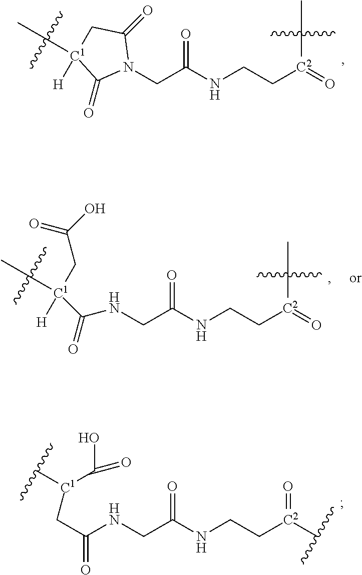

R₂ is β-D-glucose; R₃ is —NH—CH₂—O—CH₂—C(O)—; and E is exatecan;

T is flanvotumab; n is about 8; R₁ is

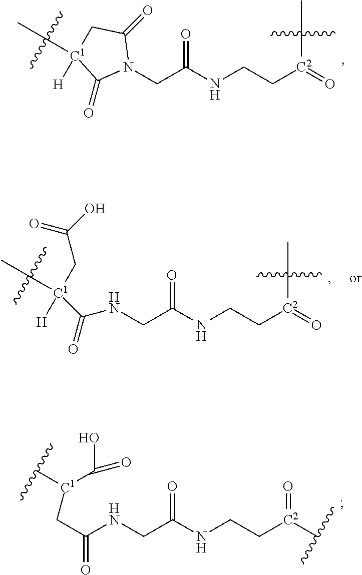

R₂ is β-D-glucuronic acid; R₃ is —NH—CH₂—O—CH₂—C(O)—; and E is exatecan;

T is flanvotumab; n is about 8; R₁ is

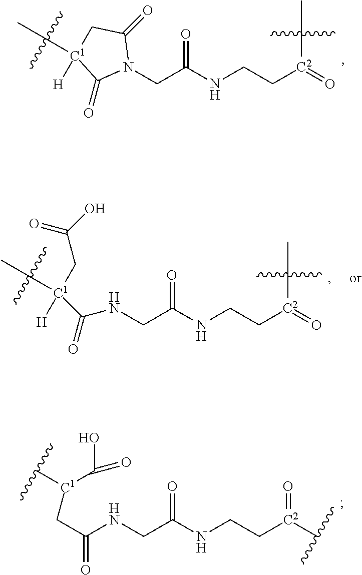

R₂ is β-D-glucose; R₃ is absent; and E is exatecan;

T is flanvotumab; n is about 8; R₁ is

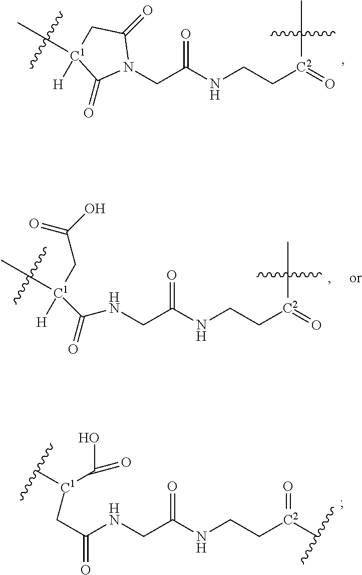

R₂ is β-D-glucuronic acid; R₃ is absent; and E is exatecan;

T is lintuzumab; n is about 8; R₁ is

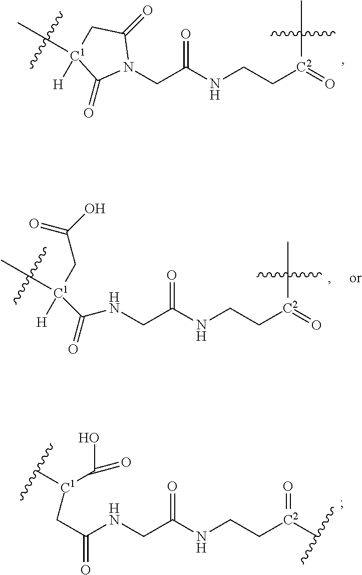

R₂ is β-D-glucose; R₃ is —NH—CH₂—O—CH₂—C(O)—; and E is exatecan;

T is lintuzumab; n is about 8; R₁ is

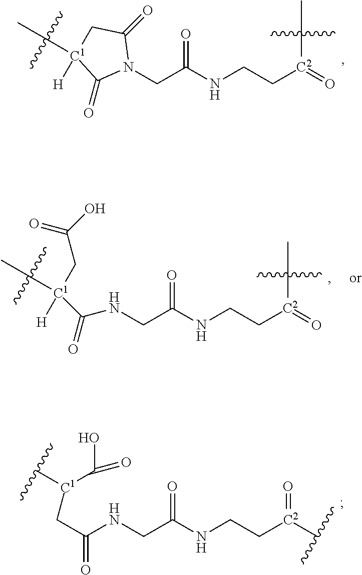

R₂ is β-D-glucuronic acid; R₃ is —NH—CH₂—O—CH₂—C(O)—; and E is exatecan;

T is lintuzumab; n is about 8; R₁ is

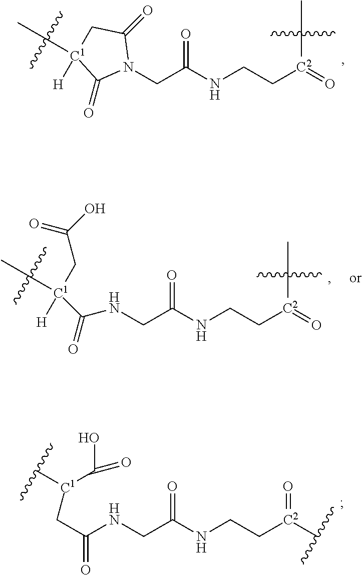

R₂ is β-D-glucose; R₃ is absent; and E is exatecan;

T is lintuzumab; n is about 8; R₁ is

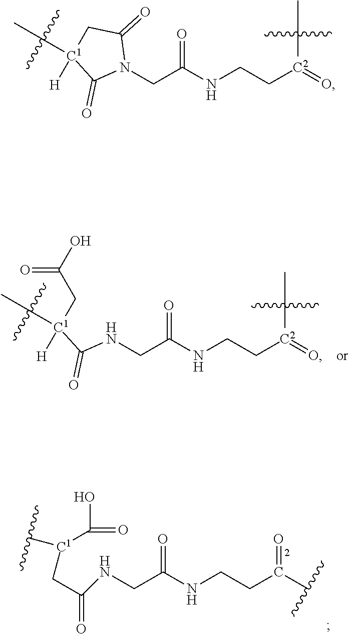

R₂ is β-D-glucuronic acid; R₃ is absent; and E is exatecan;

T is trastuzumab; n is about 8; R₁ is

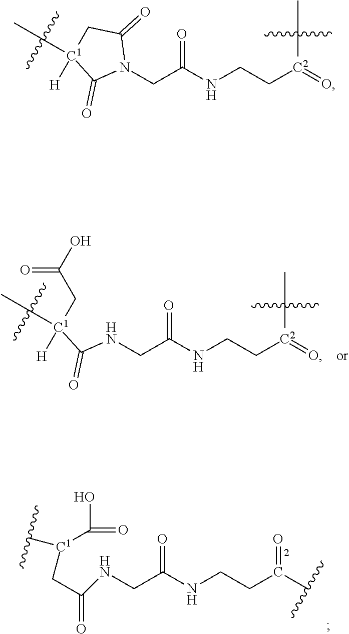

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is trastuzumab; n is about 8; R₁ is

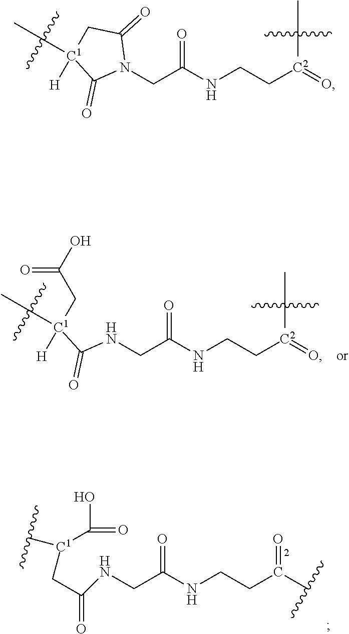

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is trastuzumab: n is about 8; R₁ is

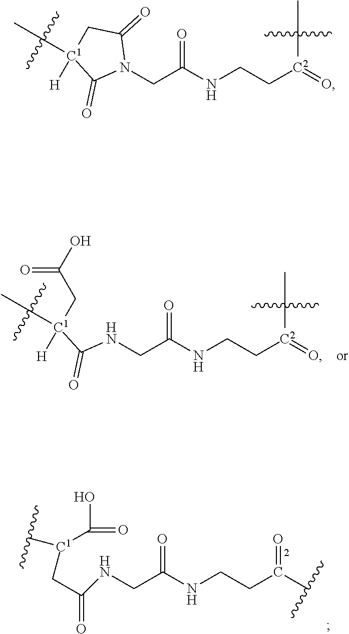

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is trastuzumab; n is about 8; R₁ is

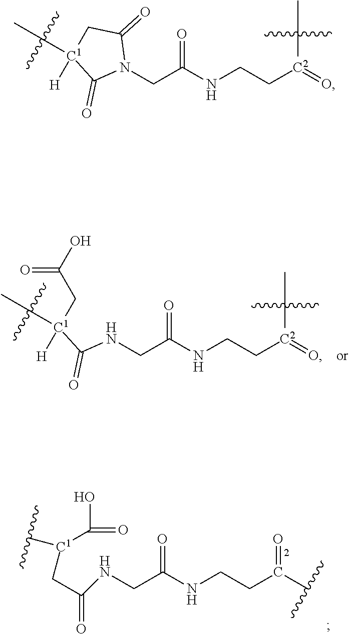

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is flanvotumab; n is about 8; R₁ is

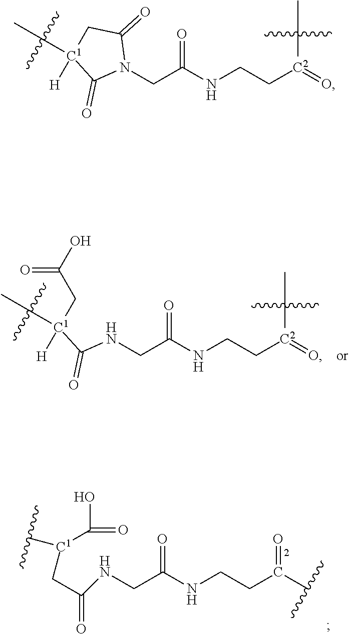

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is flanvotumab; n is about 8; R₁ is

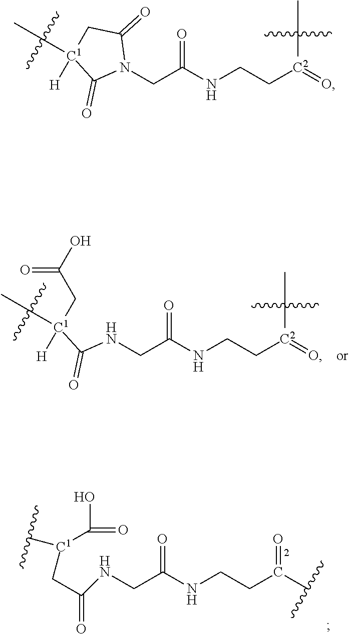

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is flanvotumab; n is about 8; R₁ is

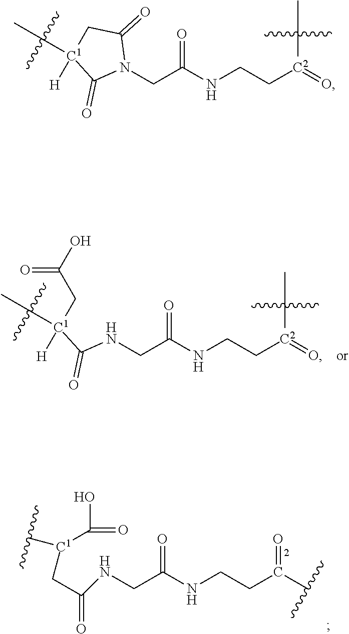

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is flanvotumab; n is about 8; R₁ is

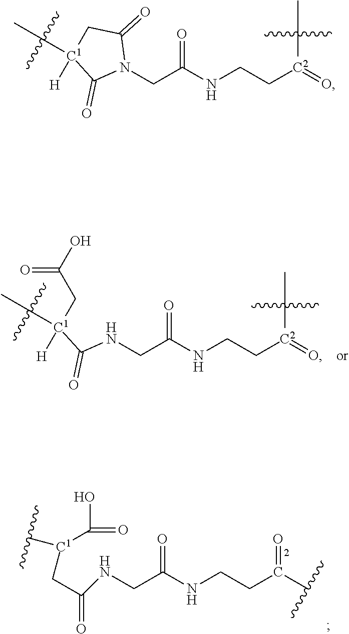

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is lintuzumab; n is about 8; R₁ is

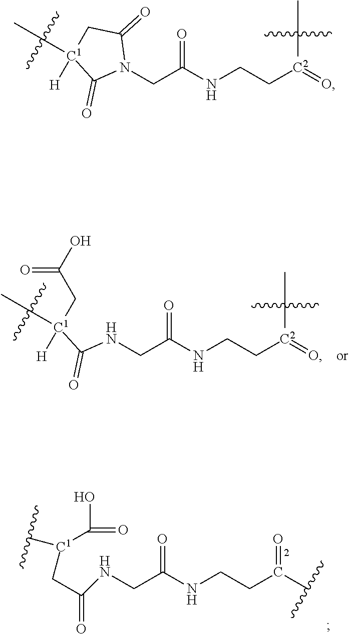

R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is lintuzumab; n is about 8; R₁ is

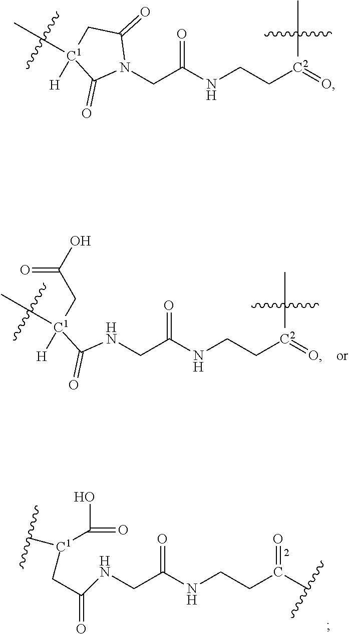

R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is lintuzumab; n is about 8; R₁ is

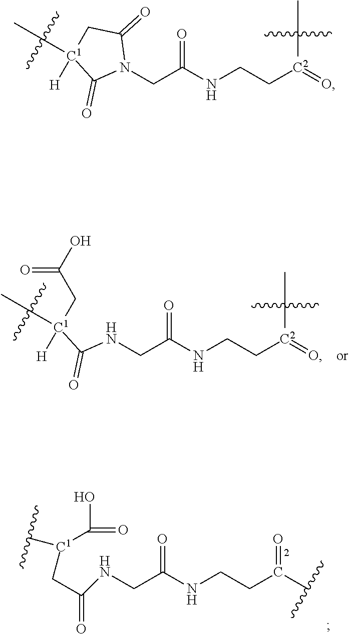

R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;

T is lintuzumab; n is about 8; R₁ is

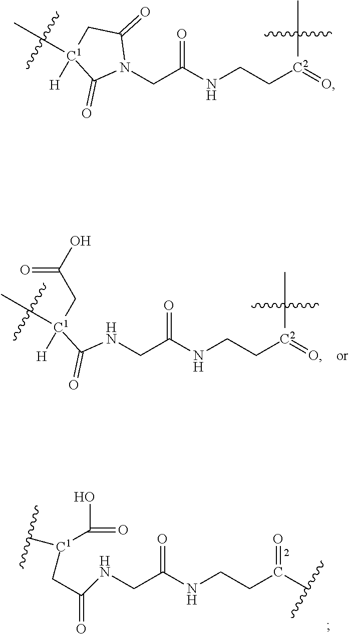

R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

A pharmaceutical composition comprising the linker-payload conjugate according to one or more embodiments or the targeting unit-linker-payload conjugate according to one or more embodiments is disclosed.

The pharmaceutical composition may further comprise a pharmaceutically acceptable carrier. Examples of suitable pharmaceutically acceptable carriers are well known in the art and may include e.g. phosphate buffered saline solutions, water, oil/water emulsions, wetting agents, and liposomes. Compositions comprising such carriers may be formulated by methods well known in the art. The pharmaceutical composition may further comprise other components such as a vehicle, an additive, a preservative, another pharmaceutical composition or pharmaceutically active compound administrated concurrently, and/or the like.

In an embodiment, the pharmaceutical composition comprises an effective amount of the linker-payload conjugate according to one or more embodiments.

In an embodiment, the pharmaceutical composition comprises an effective amount of the targeting unit-linker-payload conjugate according to one or more embodiments.

In an embodiment, the pharmaceutical composition comprises a therapeutically effective amount of the linker-payload conjugate according to one or more embodiments.

In an embodiment, the pharmaceutical composition comprises a therapeutically effective amount of the targeting unit-linker-payload conjugate according to one or more embodiments.

The term “therapeutically effective amount” or “effective amount” of the targeting unit-linker-payload conjugate should be understood as referring to the dosage regimen for modulating the growth of cancer cells and/or treating a patient's disease. The therapeutically effective amount can also be determined by reference to standard medical texts, such as the Physicians Desk Reference 2004. The patient may be male or female, and may be an infant, child or adult.

The term “treatment” or “treat” is used in the conventional sense and means attending to, caring for and nursing a patient with the aim of combating, reducing, attenuating or alleviating an illness or health abnormality and improving the living conditions impaired by this illness, such as, for example, with a cancer disease.

In an embodiment, the pharmaceutical composition comprises a composition for e.g. oral, parenteral, transdermal, intraluminal, intraarterial, intrathecal and/or intranasal administration or for direct injection into tissue. Administration of the pharmaceutical composition may be effected in different ways, e.g. by intravenous, intraperitoneal, subcutaneous, intramuscular, intratumoral, topical or intradermal administration.

As a skilled person will understand, a composition such as a pharmaceutical composition may comprise a mixture of different targeting unit-linker-payload conjugate molecules in which n is different. For example, when DAR for a pharmaceutical composition is 7.8, the pharmaceutical composition may predominantly comprise targeting unit-linker-payload conjugate molecules in which n is 8, as well as minor amounts of targeting unit-linker-payload conjugate molecules in which n is smaller than 8, for example 7 and/or 6, and possibly trace amounts of molecules in which n is smaller than 6. n, or DAR, is therefore not necessarily an integer. If the (theoretical) maximum number of payload molecules to be conjugated to the targeting unit-linker-payload conjugate molecule is 8, then the DAR should in principle not exceed 8 or about 8. The DAR may depend on e.g. the number of possible conjugation sites in the targeting unit (such as antibody), the number of payload molecules that may be conjugated to a single conjugation site, and/or the extent to which the possible conjugation sites in the targeting unit are in fact conjugated to a payload molecule.

The pharmaceutical composition may have a drug-to-antibody ratio of ≥1, or in the range of 1 to about 20, or in the range of 1 to about 15, or in the range of 1 to about 10, or in the range of 2 to 10, or in the range of 2 to 6, or in the range of 2 to 5, or in the range of 2 to 4, or in the range of about 7 to about 8; or about 1, about 2, about 3, about 4, about 5, about 6, about 7, about 8, about 9, about 10, about 11, about 12, about 13, about 14, about 15, about 16, about 17, about 18, about 19, or about 20; or in the range of about 1 to about 8, or in the range of about 6 to about 8.

In particular, in embodiments in which n is about 8 or 8 in the targeting unit-linker-payload conjugate, the pharmaceutical composition comprising the targeting unit-linker-payload conjugate may have a drug-to-antibody ratio in the range of about 7 to about 8, or in the range of about 7.5 to 8, or a drug-to-antibody ratio of about 8.

A targeting unit-linker-payload conjugate according to one or more embodiments or the pharmaceutical composition according to one or more embodiments for use as a medicament is disclosed.

A linker-payload conjugate according to one or more embodiments for use as a medicament is disclosed.

A targeting unit-linker-payload conjugate according to one or more embodiments or the pharmaceutical composition according to one or more embodiments for use in the treatment of cancer is disclosed.

A linker-payload conjugate according to one or more embodiments for use in the treatment of cancer is disclosed.

In an embodiment, the cancer is selected from the group consisting of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma and testicular cancer.

A method of treating and/or modulating the growth of and/or prophylaxis of tumor cells in humans or animals is disclosed, wherein the linker-payload conjugate according to one or more embodiments, targeting unit-linker-payload conjugate according to one or more embodiments or the pharmaceutical composition according to one or more embodiments is administered to a human or animal in an effective amount.

In an embodiment, the tumor cells are selected from the group consisting of leukemia cells, lymphoma cells, breast cancer cells, prostate cancer cells, ovarian cancer cells, colorectal cancer cells, gastric cancer cells, squamous cancer cells, small-cell lung cancer cells, head-and-neck cancer cells, multidrug resistant cancer cells, and testicular cancer cells.

A method of treating cancer in humans is disclosed, wherein the linker-payload conjugate according to one or more embodiments, the targeting unit-linker-payload conjugate according to one or more embodiments or the pharmaceutical composition according to one or more embodiments is administered to a human in an effective amount.

In an embodiment, the effective amount is a therapeutically effective amount.

In an embodiment, the linker-payload conjugate according to one or more embodiments, the targeting unit-linker-payload conjugate according to one or more embodiments or the pharmaceutical composition according to one or more embodiments is administered intravenously to a human in a therapeutically effective amount.

In an embodiment, the linker-payload conjugate according to one or more embodiments, the targeting unit-linker-payload conjugate according to one or more embodiments or the pharmaceutical composition according to one or more embodiments is administered intratumorally to a human in a therapeutically effective amount.

In an embodiment, the cancer is selected from the group consisting of head-and-neck cancer, leukemia, lymphoma, breast cancer, prostate cancer, ovarian cancer, colorectal cancer, gastric cancer, squamous cancer, small-cell lung cancer, multidrug resistant cancer and testicular cancer.

The embodiments described hereinbefore may be used in any combination with each other. Several of the embodiments may be combined together to form a further embodiment. A product or a method to which the present disclosure is related may comprise at least one of the embodiments described hereinbefore.

The linker-payload conjugate according to one or more embodiments and the targeting unit-linker-payload conjugate according to one or more embodiments may have a number of beneficial properties.

The presence of the cleavable hydrophilic group renders the otherwise relatively poorly water-soluble linker more soluble in aqueous and physiological solutions. The improved solubility also improves the retention of the targeting unit-linker-payload conjugate in serum. It may also have high uptake in cells to which it is targeted and low uptake in cells and organs to which it is not targeted.

The targeting unit-linker-payload conjugate according to one or more embodiments is less toxic in the absence or low activity of lysosomal and intracellular enzymes. Since cancer cells typically display high activity of lysosomal and/or intracellular enzymes, the toxic payload moiety is preferentially released in cancer cells as compared to non-cancer cells.

The conjugate has low antigenicity.

The targeting unit-linker-payload conjugate according to one or more embodiments also exhibits good pharmacokinetics. It has suitable retention in blood, high uptake in cells to which it is targeted and low uptake in cells and organs to which it is not targeted.

The targeting unit-linker-payload conjugate according to one or more embodiments is sufficiently stable towards chemical or biochemical degradation during manufacturing or in physiological conditions, e.g. in blood, serum, plasma or tissues.

As shown in Example 9, free exatecan payload (according to Formula E1) is generally more cytotoxic against cancer cells than free DXd payload (according to Formula E2), and an ADC with the exatecan payload is generally more cytotoxic against cancer cells than an ADC with the DXd payload. Therefore, the targeting unit-linker-payload conjugates according to one or more embodiments, which comprise the exatecan payload instead of the DXd payload, may have increased anti-cancer activity potential.

Even relatively minor differences in the structure of targeting unit-linker-payload conjugates may have unexpected technical effects. For example, targeting unit-linker-payload conjugates represented by Formula IB may show a higher activity as compared to targeting unit-linker-payload conjugates represented by Formula I.

### EXAMPLES

In the following, the present invention will be described in more detail. Reference will now be made in detail to the embodiments, examples of which are illustrated in the accompanying drawings. The description below discloses some embodiments in such detail that a person skilled in the art is able to utilize the invention based on the disclosure. Not all steps of the embodiments are discussed in detail, as many of the steps will be obvious for the person skilled in the art based on this specification.

General equipment and reagents include:
HPLC: Äkta Purifier 100 and 10 (GE Healthcare)
HiTrap MabSelect SuRe protein A column 1 ml (GE Healthcare)
HiTrap Desalting column 5 ml (GE Healthcare)
Loading buffer: 20 mM Na-phosphate pH 7.2, 150 mM NaCl, or PBS (Lonza)
Elution buffer: 0.1 M citrate pH 3.0
Desalting buffer: PBS, Gibco
Spectrophotometer: Nanodrop one (Thermo Fisher Scientific)
TCEP: Tris-2-carboxyethyl phosphine hydrochloride
DMSO: Dimethyl sulfoxide
TFA: Trifluoroacetic acid
DHAP: 2′, 5′, dihydroxyacetophenone
BSA: Bovine serum albumin
MALDI-TOF mass spectrometer: Bruker Ultraflex III
Superdex 200 column (10×300 mm, GE Healthcare)
TSKgel ButylNPR column (4.6 mm×3.5 cm, Tosoh Biosciences)
PLRP-S column (1000 Å, 8 μM, 150×2.1 mm, Agilent)
SDS-PAGE: 4-15% Mini-Protean TGX gel (Bio-Rad), Laemmli running buffer, Spectra multicolor broad range protein ladder (Thermo Scientific), Imperial protein stain (Pierce Thermo Scientific).

### Example 1

### Generation of Flanvotumab-Exatecan ADCs

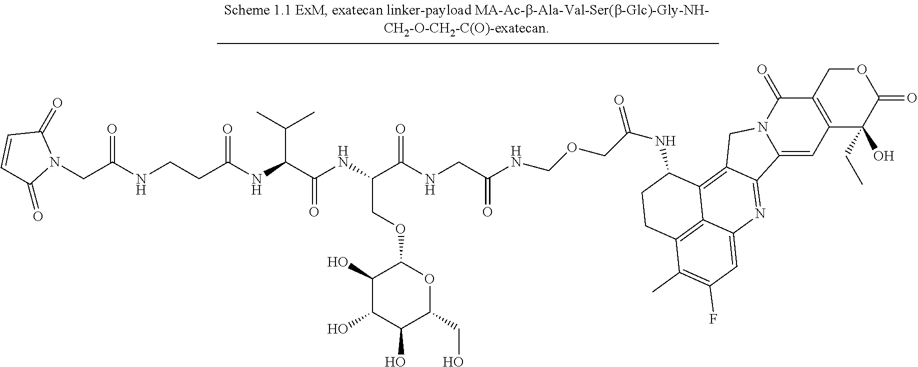 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](CO[C@@H]1O[C@H](CO)[C@@H](O)[C@H](O)[C@H]1O)NC(=O)[C@@H](NC(=O)CCNC(=O)CN1C(=O)C=CC1=O)C(C)C)CC3]

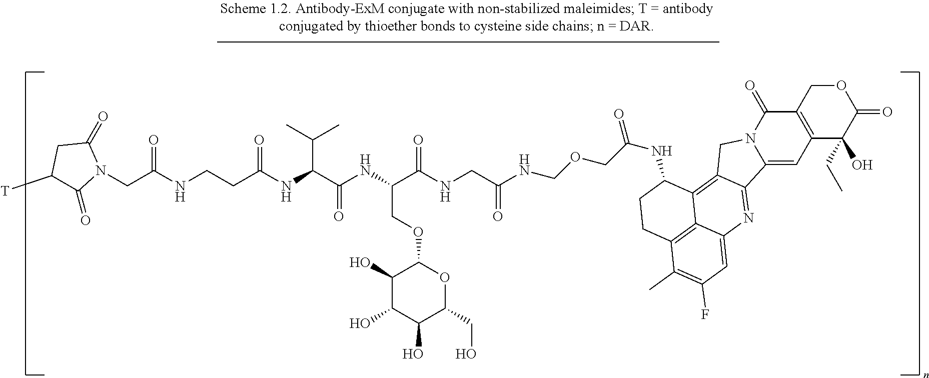

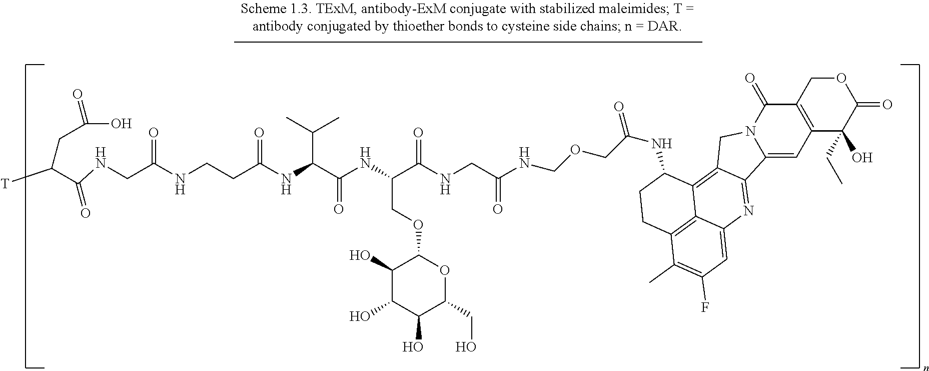

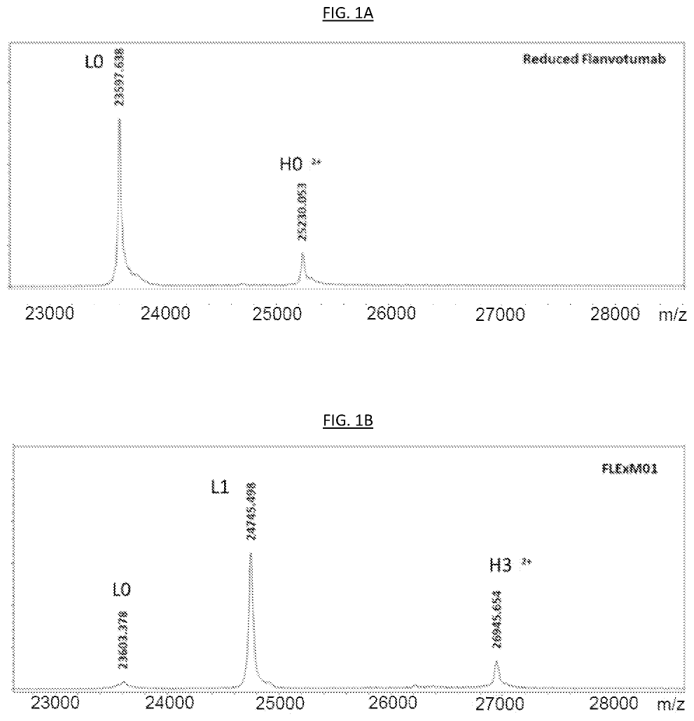

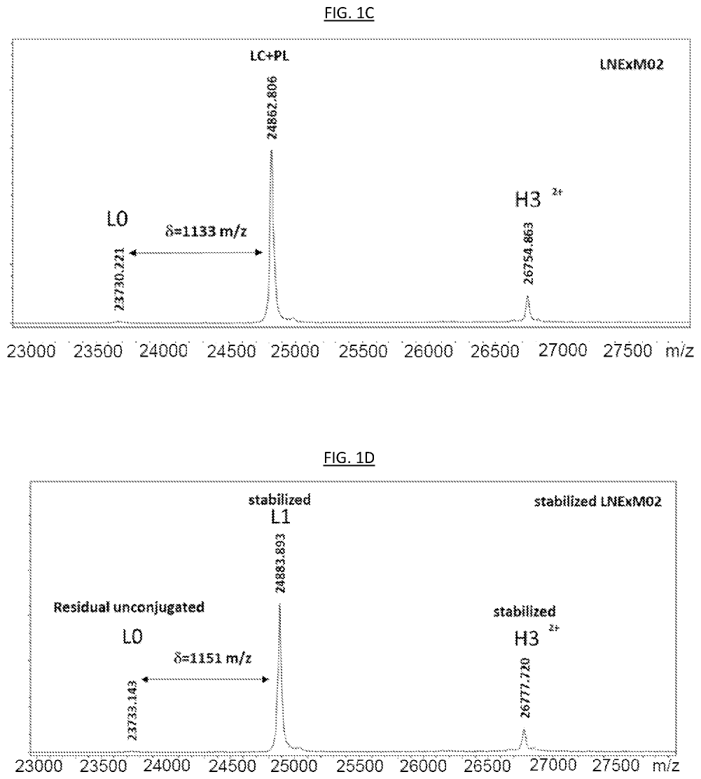

FLExM01: 9 mg of Flanvotumab (FL) was diluted to 2.0 mg/ml with PBS and was then reduced in the presence of 25× molar excess of TCEP at +37° C. for one hour. 30× molar excess of ExM: MA-Ac-β-Ala-Val-Ser (β-Glc)-Gly-NH—CH₂—O—CH₂—C(O)— exatecan (Scheme 1.1) was added and reaction allowed to proceed at +37° C. for 1.5 hours. Extent of conjugation was checked using MALDI-TOF MS essentially as in Satomaa et al. (2018) Antibodies 7(2): 15, and the diluted reaction mixture was mixed with 2% TFA and DHAP matrix and applied on MALDI plate into the mass spectrometer. The analysis suggested Drug-to-Antibody Ratio (DAR) close to 8, or DAR≈8 (Scheme 1.2, FIG. 1A-B).

The ADC was purified with HiTrap MabSelect Sure column (1 ml, GE Healthcare) in Äkta HPLC purifier system. Sample was loaded to column and washed with 12-14 column volumes of loading buffer. 5 column volumes of 0.1 M citrate pH 3.0 was used for elution. After elution, buffer of antibody sample was changed for PBS using HiTrap Desalting column (5 ml, GE Healthcare). Concentrations were determined by spectrophotometer (Nanodrop one, Thermo Fisher Scientific). The purified ADC was sterile filtered, and the yield was 5.3 mg.

The purified ADC self-stabilized by maleimide hydrolysis in the storage formulation of PBS pH 7.4 (Scheme 1.3), yielding a more stable ADC than with deruxtecan ADCs that do not similarly self-stabilize.

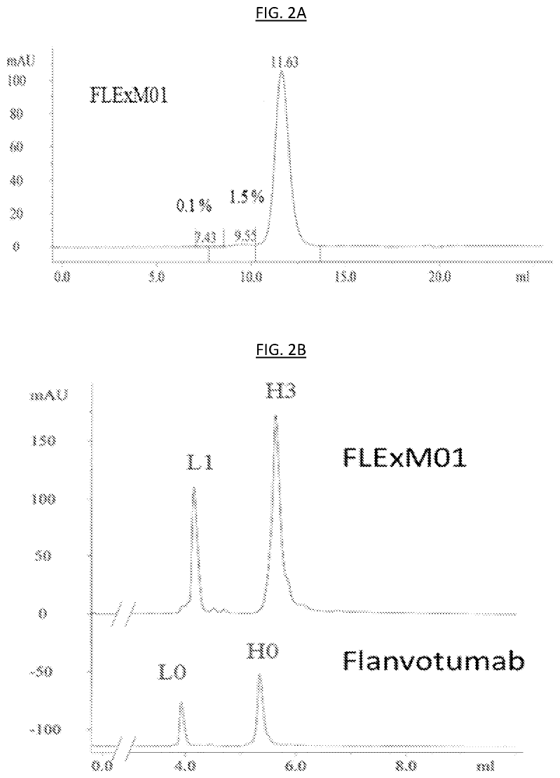

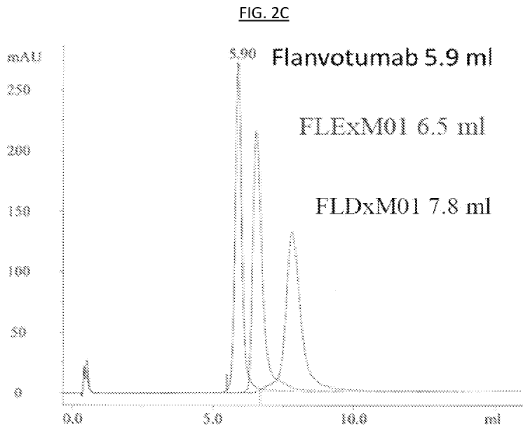

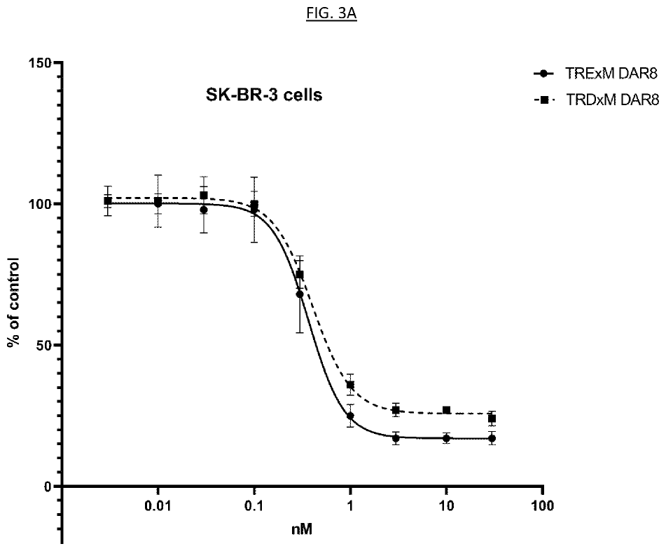

Aggregation status of FLExM01 was evaluated by size-exclusion chromatography (SEC-HPLC; FIG. 2A), showing very low aggregation with high-molecular weight aggregate level of 1.6%. Drug-to-Antibody Ratio (DAR) was determined by reversed-phase chromatography (RP-HPLC; FIG. 2B), showing homogeneous conjugation to DAR=8.

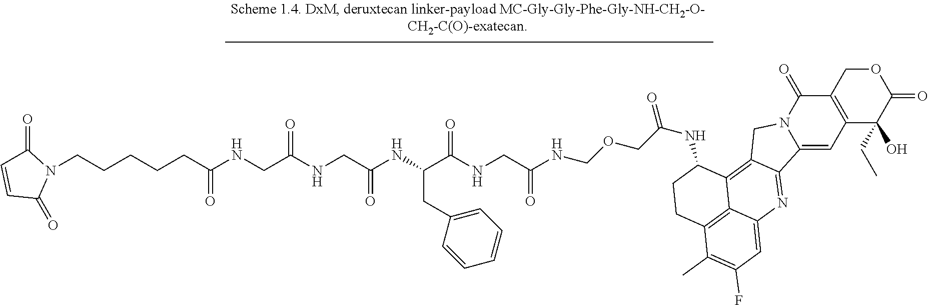 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

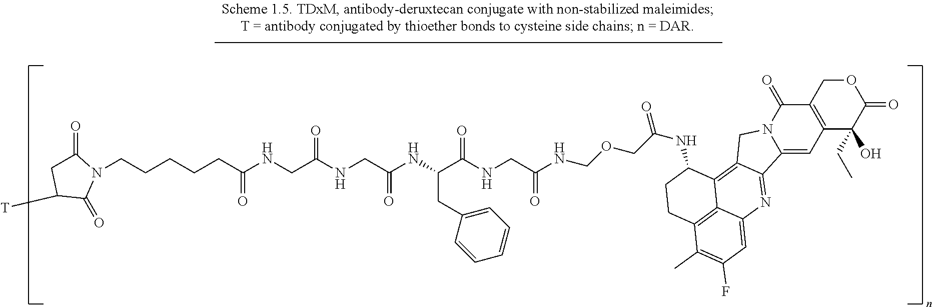

Flanvotumab-deruxtecan ADC was generated essentially similarly as FLExM above using deruxtecan linker-payload (DxM, MedChemExpress, Sweden; Scheme 1.4). The FLDxM01ADC had DAR≈8 (Scheme 1.5) and low aggregates according to MALDI-TOF MS, SEC-HPLC and RP-HPLC similarly as above for FLDxM01. However, HIC-HPLC analysis showed that FLExM was more hydrophilic than FLDxM, in other words FLExM eluted markedly closer to Flanvotumab than FLDxM in HIC (FIG. 2C). Higher hydrophilicity is beneficial to the in vivo pharmacokinetics, efficacy and tolerability of ADCs indicating that ADC with the ExM linker-payload has better in vivo properties than ADC with the deruxtecan linker-payload.

### Example 2

### Generation of Lintuzumab-Exatecan ADCs

20 mg of humanized recombinant antibody Lintuzumab was produced in CHO cells by transient expression, purified essentially as above and used in 3.38 mg/ml concentration in PBS. Lintuzumab was reduced in the presence of 20× molar excess of TCEP at +37° C. for 1.5 hours. The reaction was followed by MALDI-TOF MS as above. For LNExM02 synthesis, 28× molar excess of ExM linker-payload was added, and reaction allowed to proceed at +37° C. for 1.5 hours. Extent of conjugation was checked using MALDI-TOF MS (FIG. 1C; Scheme 1.2). The analysis suggested high DAR close to 8. The conjugated maleimide rings were directly stabilized by incubating the ADC at 37° C. for 24 hours. Stabilization was confirmed using MALDI-TOF MS (FIG. 1D; Scheme 1.3). Stabilization was performed with purified sample prior to sterile filtration. The payload-conjugated light chain fragment L1 was used to track the hydrolysis reaction (the molecular mass of the payload increased by 18 Da indicating that the maleimide ring was hydrolyzed during incubation). The purified LNExM02 ADC was sterile filtered.

The corresponding lintuzumab-deruxtecan ADC LNDxM DAR=8 was generated essentially similarly as above, however the maleimides are not stabilized (Scheme 1.5).

### Example 3

### Generation of Trastuzumab-Exatecan ADCs

Trastuzumab-exatecan ADC DAR=8 (TRExM; Scheme 1.3) and the corresponding trastuzumab-deruxtecan ADC DAR=8 (TRDxM; Scheme 1.5) were generated essentially similarly as above and characterized by MALDI-TOF MS, SEC-HPLC and HIC-HPLC essentially as above as homogeneous ADC products.

### Example 4

### In Vitro Cytotoxicity Assays

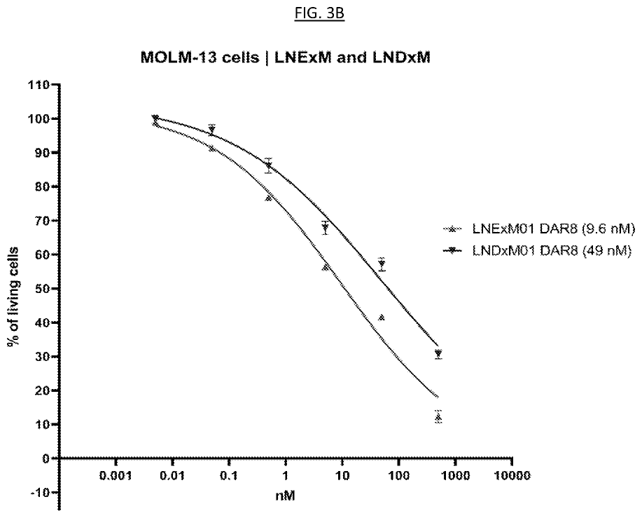

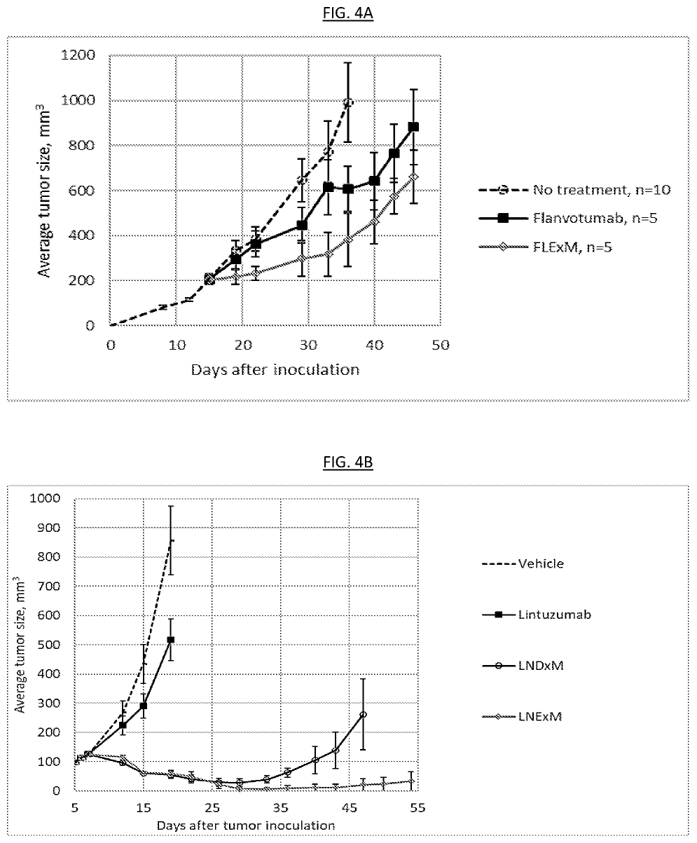

HER2+ SK-BR-3 breast cancer cells (ATCC) and CD33+ MOLM-13 leukemia cells (DSMZ, German Collection of Microorganisms and Cell Cultures) were cultured essentially according to the providers' instructions. FIG. 3 shows in vitro cytotoxicity assays of the different exatecan ADCs and free exatecan. FIG. 3A: HER2+ SK-BR-3 breast cancer cells were incubated with trastuzumab-exatecan ADC TRExM01, trastuzumab-deruxtecan ADC TRDxM01 and free exatecan payload (provided as mesylate salt). IC50 values are shown in the figure and they showed that TRExM01 with the ExM linker-payload DAR=8 was more cytotoxic to the HER2+ cells than TRDxM01 with the deruxtecan linker-payload DAR=8. FIG. 3B: CD33+ MOLM-13 leukemia cells were incubated with lintuzumab-exatecan ADC LNExM01 and lintuzumab-deruxtecan ADC TRDxM01. IC50 values are shown in the figure and they showed that LNExM01 with the ExM linker-payload DAR=8 was more cytotoxic to the CD33+ leukemia cells than LNDxM01 with the deruxtecan linker-payload DAR=8.

### Example 5

### Tumor Xenograft Models

SK-MEL-30 human melanoma xenograft model (FIG. 4A): The animal model were performed at the University of Helsinki, Finland, according to the appropriate ethical committee approval. SK-MEL-30 melanoma cells, 2 million cells/mouse, were inoculated subcutaneously (s.c.) to athymic mice. Tumor take was high, and the mice were divided to groups with similar average tumor sizes when they reached about 200 mm³. FIG. 4A shows tumor growth in the SK-MEL-30 xenograft model with single i.v. dose of either 10 mg/kg Flanvotumab antibody (n=5) or 10 mg/kg FLExM ADC (n=5) compared to control mice that received no treatment (n=10). Tumor growth was inhibited markedly more in the ADC-treated mice than either the antibody-treated or non-treated mice, showing that the anti-TYRP-1 ADC had clear therapeutic effect. No toxicities were observed.

MOLM-13 human melanoma xenograft model (FIG. 4B): In vivo anti-leukemia xenograft efficacy was evaluated for Lintuzumab antibody, LNExM ADC (DAR=8) and LNDxM ADC (DAR=8). The study was performed at the TCDM, University of Turku, Finland, according to the appropriate ethical committee approval. Cells for inoculation to mice were prepared in vigorous exponential growth phase. 2 million MOLM-13 cells in 50% Matrigel were inoculated s.c. to the flank of each mouse (female athymic nude mice between 8-10 weeks of age). Clinical signs and general behavior of the animals were observed regularly. No signs of toxicity were recorded. At the end of the study, the mice were examined for potential macroscopic changes in major organs, but none were detected. Tumor growth was followed by palpation. After caliper measurement, tumor volume was calculated according to 0.5×length×width². Tumor take was excellent, and the mice were divided to groups with similar average tumor sizes when they reached about 120 mm³, and the dosings were administered. Mice were evenly divided into study groups, 6 mice in each group, so that each group received similar distribution of different-sized tumors and the average tumor volumes were similar in each group. Intravenous (i.v.) treatment in PBS was given once on day 1 of treatment as a single dose regimen (on day 8 after tumor inoculation). The experiment lasted to day 46 after treatment (day 54 after tumor inoculation).

FIG. 4B shows the results of the MOLM-13 xenograft study. While Lintuzumab antibody had only slight inhibitory activity to tumor growth, LNExM was even more effective than LNDxM in reducing the average tumor volumes. One mice died due to tumor growth in the LNDxM treatment group on day 47 after tumor inoculation (⅙, 17%), whereas no deaths occurred in LNExM treatment group. In addition, with LNExM treatment the tumors disappeared and did not regrow during the course of the whole study in 5/6 mice (83% tumor-free mice at the end of the study), while only 2/6 mice were tumor-free mice at the end of the study with LNDxM treatment (33%). In conclusion, LNExM with the ExM linker-payload was markedly more effective than LNDxM with the deruxtecan linker-payload in this in vivo model.

### Example 6

### Generation of MA-Ac-β-Ala-Val-Ser(β-GlcA)-PABC-Exatecan ADC

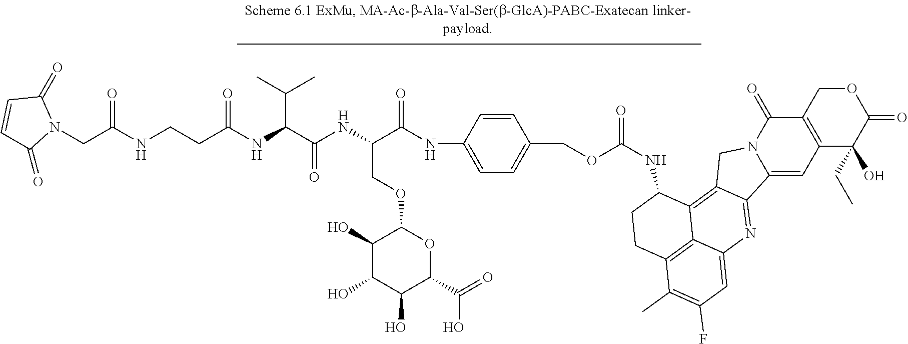 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CO[C@@H]2O[C@H](C(=O)O)[C@@H](O)[C@H](O)[C@H]2O)NC(=O)[C@@H](NC(=O)CCNC(=O)CN2C(=O)C=CC2=O)C(C)C)cc1)CC3]

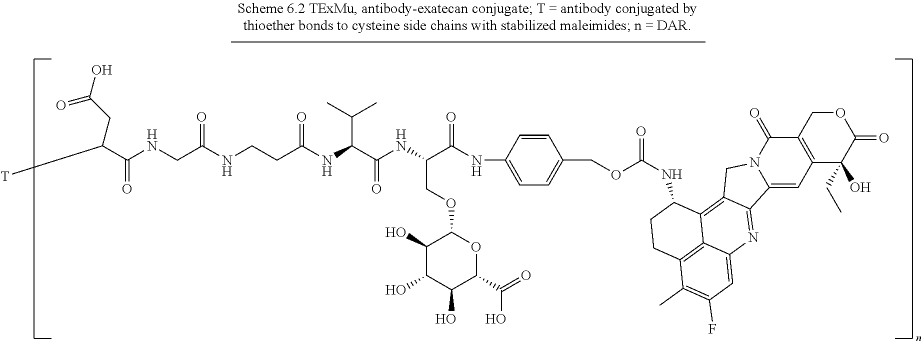

To generate LNExMu ADC (Scheme 6.2) from lintuzumab antibody and ExMu linker-payload that comprises the PABC self-immolative group directly C-terminal to the glycoserine residue (Scheme 6.1), 1 mg of lintuzumab at concentration of 3.4 mg/ml in PBS was reduced in the presence of 20× molar excess of TCEP at +37°° C. for 1.5 hours and combined with 25× molar excess of ExMu in DMSO, and reaction allowed to proceed at +37° C. for 2 hours. Extent of conjugation was checked using MALDI-TOF MS as above, and the DAR was between 6-7. Additional 4.4× molar excess of ExMu was added and incubated as above, but no obvious improvement in DAR was observed in MS. The ADC was purified as described above, but the resulting ADC preparate had a very low concentration of 0.17 mg/ml and yield showing that the majority of the ADC had been lost during the purification. Thus LNExMu could not be efficiently generated with the same process than LNExM.

Next it was studied whether Tris buffer would be better in conjugation. 2 mg of lintuzumab in 50 mM Tris-HCl pH 7.5 was reduced in the presence of 20× molar excess of TCEP at +37° C. for 1.5 hours. This was followed by 25× molar excess of ExMu, and the reaction was allowed to proceed at +37° C. for 2 hours. Extent of conjugation was checked using MALDI-TOF MS and the DAR was still less than 8 and both non-conjugated light chain as well as heavy chain with only two payloads were abundant. In addition, the HPLC purification of the ADC again led to loss of the majority of the ADC and low concentration of the eluate. Concentration was attempted, but it led to precipitation when the volume was reduced. It was concluded that the aqueous solubility of the LNExMu ADC was markedly lower than with LNExM ADC, with which no precipitation had been observed at concentrations of over a magnitude higher in similar conditions.

A larger amount of LNExMu was produced from 3 mg of lintuzumab that was reduced in the presence of 20× molar excess of TCEP at +37° C. for 1.5 hours. The reaction was followed by MALDI-TOF MS as above. For LNExMu synthesis, 25× molar excess of ExMu in DMSO (Scheme 6.1) was added, and reaction allowed to proceed at +37° C. for 2 hours. Extent of conjugation was checked using MALDI-TOF MS and this time the DAR was close to 8. However, all purification and concentration attempts yielded a maximum concentration of 0.36 mg ADC/ml.

### Example 7

### Relative ADC Hydrophilicity Evaluation by HIC

HIC-HPLC analysis using TSKgel ButylNPR column was done with Äkta HPLC purifier system. ADC or antibody was injected to the column and separated by gradient elution: 100% buffer A (1.5 M ammoniumsulfate, 25 mM K-phosphate) to 100% buffer B (25% isopropanol, 25 mM K-phosphate) for 15 minutes (1 mL/min) continued with 100% B for 2 minutes. Table below shows relative elution times of different exatecan ADCs as % compared to the parent antibody. It was evident that LNExMu had a higher hydrophobicity than LNExM, correlating with its higher tendency to precipitate at moderate concentrations from aqueous solution as well as its low yield in HPLC purification. In conclusion, LNExM was more hydrophilic and more feasible to produce in larger quantities and higher concetration than LNExMu, while LNDxM most the most hydrophobic of the tested ADCs.

TABLE 1
Relative HIC-HPLC elution times of Lintuzumab,
LNExM, LNExMu and LNDxM.

| Antibody or | Elution time relative to | Relative hydrophilicity ranking |
|---|---|---|
| ADC batch | parent antibody, % | (1 = most hydrophilic) |
| Lintuzumab | 100% | 1 = Lintuzumab |
| LNExM01 | 109% | 2 = LNExM |
| LNExM02 | 110% | |
| LNExMu01 | 116% | 3 = LNExMu |
| LNDxM01 | 124% | 4 = LNDxM |
| LNDxM02 | 124% | |
| LNDxM03 | 124% | |

### Example 8

### Generation of MA-Ac-β-Ala-Val-Ser(β-Glc)-Exatecan, MA-Ac-β-Ala-Val-Ser(β-Glc)-Gly-Exatecan and MA-Ac-β-Ala-Val-Ser(β-Glc)-β-Ala-Exatecan ADCs.

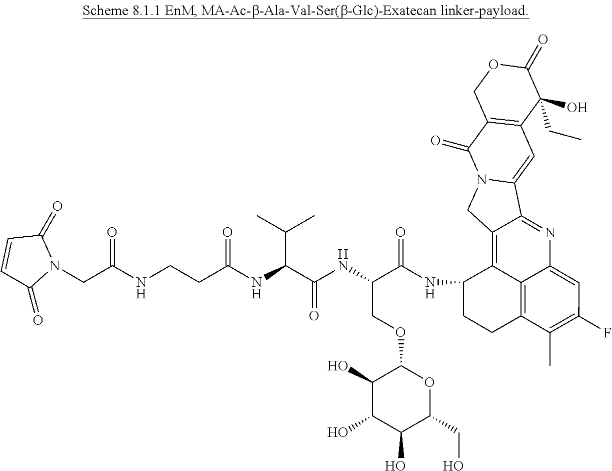 [SMILES: CC[C@@]1(O)C(=O)OCc2c1cc1n(c2=O)Cc2c-1nc1cc(F)c(C)c3c1c2[C@@H](NC(=O)[C@H](CO[C@@H]1O[C@H](CO)[C@@H](O)[C@H](O)[C@H]1O)NC(=O)[C@@H](NC(=O)CCNC(=O)CN1C(=O)C=CC1=O)C(C)C)CC3]

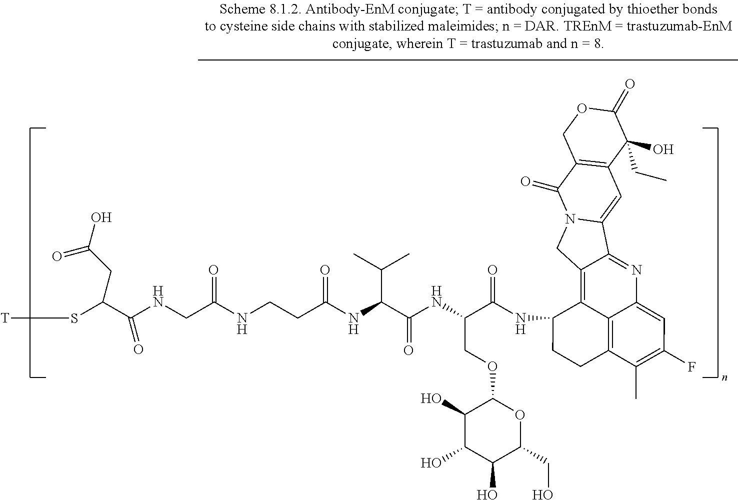

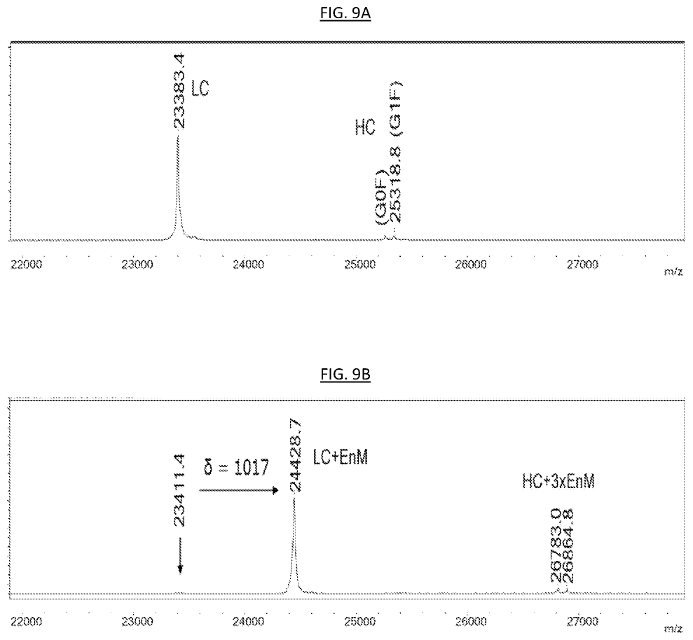

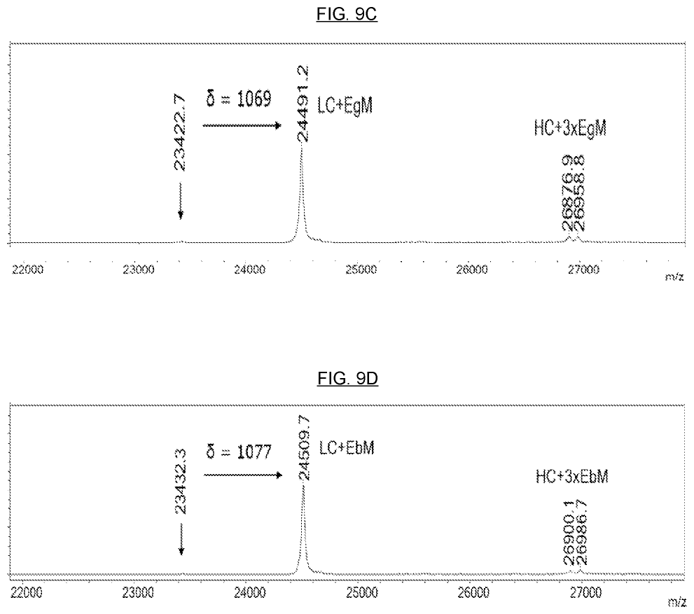

Generation of TREnM ADC (Scheme 8.1.2): The EnM linker-payload (Scheme 8.1.1) was synthesized otherwise similarly as the linker-payloads described above, but with the glucoserine carboxylic acid group of the linker moiety directly amidated to the primary amine group of the exatecan payload. The TREnM ADC was produced essentially as described above from trastuzumab that was reduced in the presence of a molar excess of TCEP at +37°° C. in PBS buffer and conjugated by adding a molar excess of EnM in DMSO. The extent of conjugation was checked using MALDI-TOF MS and the DAR was 8 (FIG. 9B). The purification yielded 5.4 mg of ADC, 4.7 mg/ml in PBS buffer.

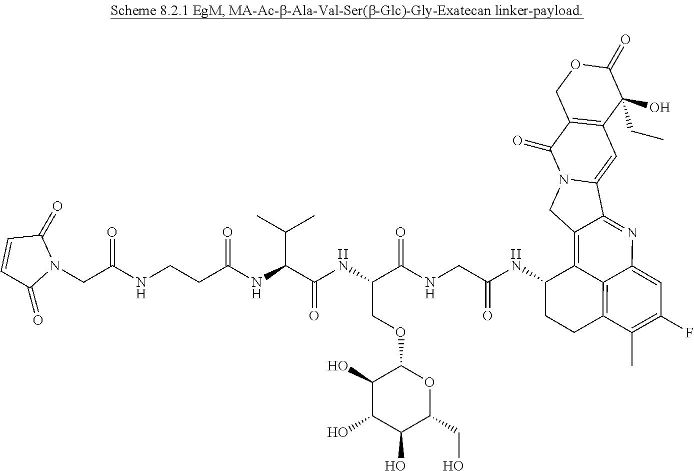 [SMILES: CC[C@@]1(O)C(=O)OCc2c1cc1n(c2=O)Cc2c-1nc1cc(F)c(C)c3c1c2[C@@H](NC(=O)CNC(=O)[C@H](CO[C@@H]1O[C@H](CO)[C@@H](O)[C@H](O)[C@H]1O)NC(=O)[C@@H](NC(=O)CCNC(=O)CN1C(=O)C=CC1=O)C(C)C)CC3]

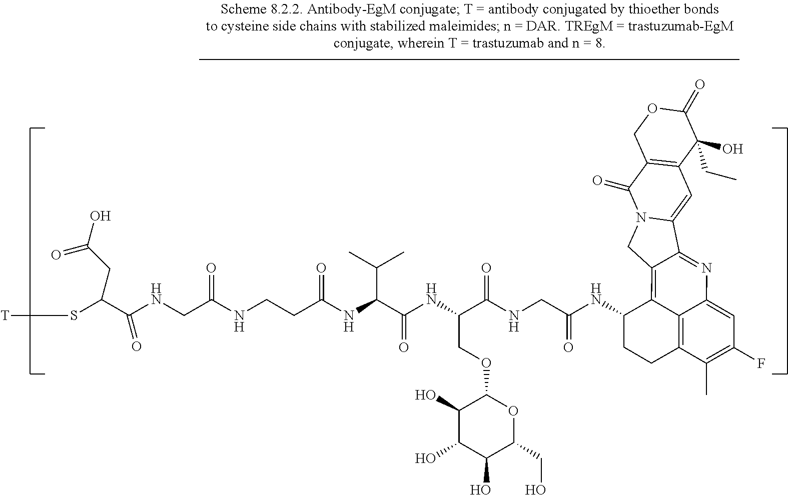

Generation of TREgM ADC (Scheme 8.2.2): The EgM linker-payload (Scheme 8.2.1) was synthesized otherwise similarly as the linker-payloads described above, but with the glycine carboxylic acid group of the linker moiety directly amidated to the primary amine group of the exatecan payload. The TREgM ADC was produced essentially as described above from trastuzumab that was reduced in the presence of a molar excess of TCEP at +37° C. in PBS buffer and conjugated by adding a molar excess of EgM in DMSO. The extent of conjugation was checked using MALDI-TOF MS and the DAR was 8 (FIG. 9C). The purification yielded 5.4 mg of ADC, 4.7 mg/ml in PBS buffer.

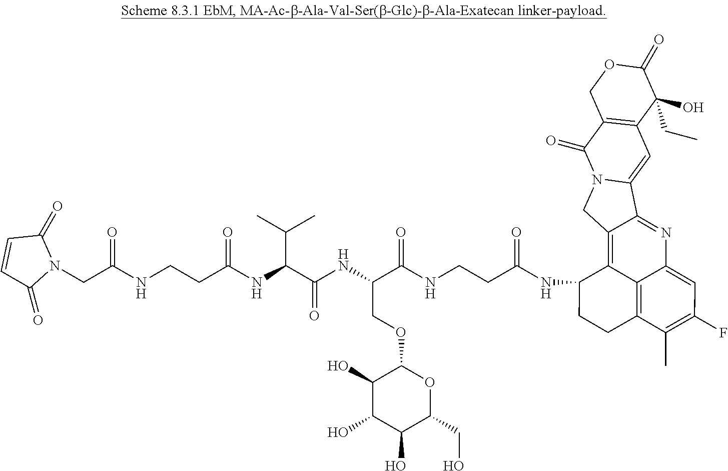 [SMILES: CC[C@@]1(O)C(=O)OCc2c1cc1n(c2=O)Cc2c-1nc1cc(F)c(C)c3c1c2[C@@H](NC(=O)CCNC(=O)[C@H](CO[C@@H]1O[C@H](CO)[C@@H](O)[C@H](O)[C@H]1O)NC(=O)[C@@H](NC(=O)CCNC(=O)CN1C(=O)C=CC1=O)C(C)C)CC3]

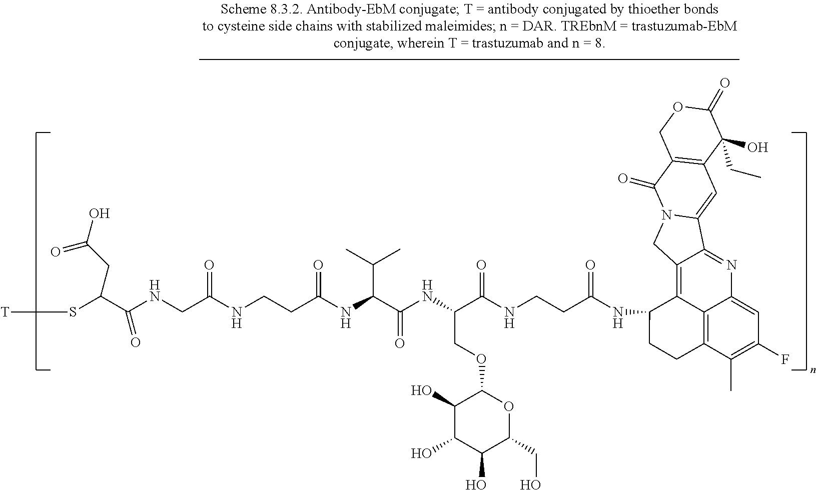

Generation of TREbM ADC (Scheme 8.3.2): The EbM linker-payload (Scheme 8.3.1) was synthesized otherwise similarly as the linker-payloads described above, but with the C-terminal β-alanine carboxylic acid group of the linker moiety directly amidated to the primary amine group of the exatecan payload. The TREbM ADC was produced essentially as described above from trastuzumab that was reduced in the presence of a molar excess of TCEP at +37° C. in PBS buffer and conjugated by adding a molar excess of EbM in DMSO. The extent of conjugation was checked using MALDI-TOF MS and the DAR was 8 (FIG. 9D). The purification yielded 5.3 mg of ADC, 4.6 mg/ml in PBS buffer.

### Example 9

### In Vitro Cytotoxicity Assays

HER2+ SK-BR-3 breast cancer cells (ATCC), HCC1954 breast cancer cells (CRL-2338, ATCC), JIMT-1 breast cancer cells (DSMZ) and SKOV-3 ovarian cancer cells (ATCC) were cultured essentially according to the providers' instructions. After incubation with ADCs, cell viability was determined using PrestoBlue reaction using manufacturer's instructions. Cell viability data data was transferred to GraphPad Prism to determine IC50 values. Exatecan (mesylate salt) and DXd (free deruxtecan payload) were purchased from MedChemExpress.

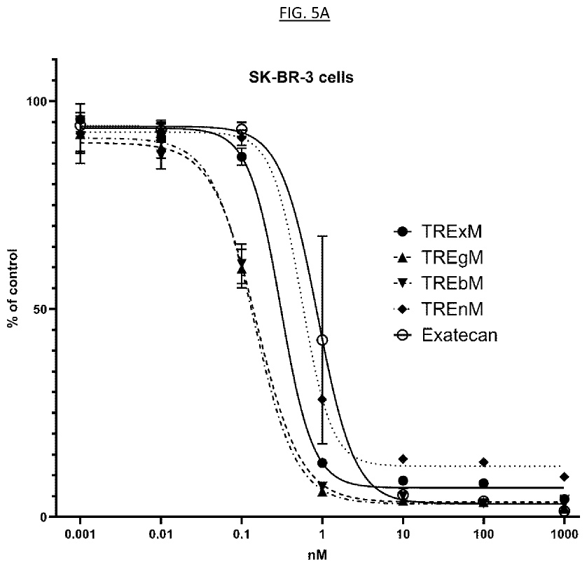

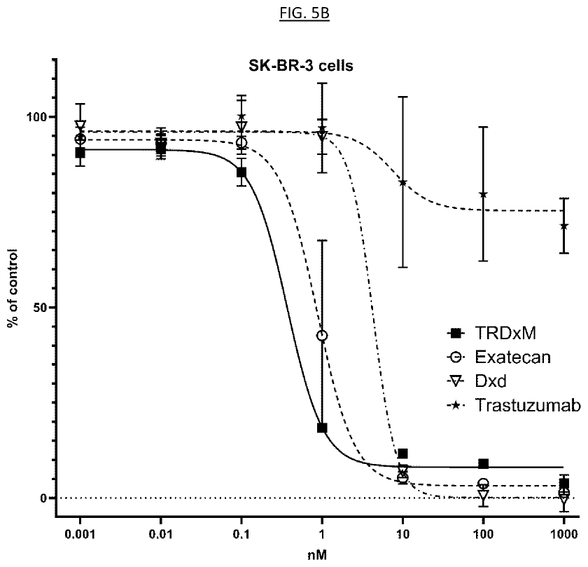

FIGS. 5-8 show in vitro cytotoxicity assays of the exatecan ADCs, free exatecan, free DXd and trastuzumab.

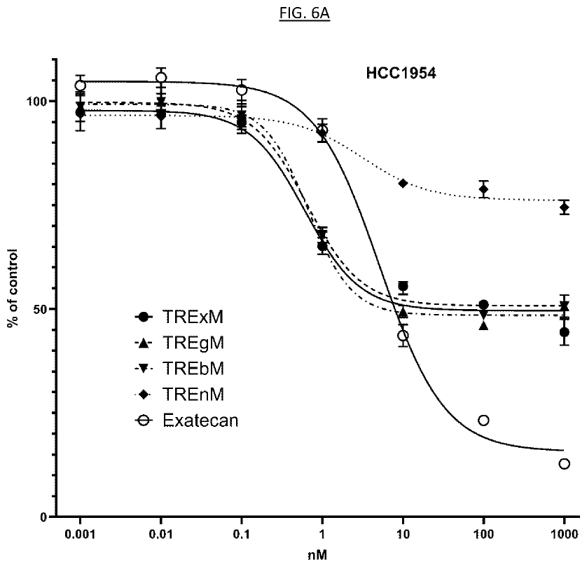

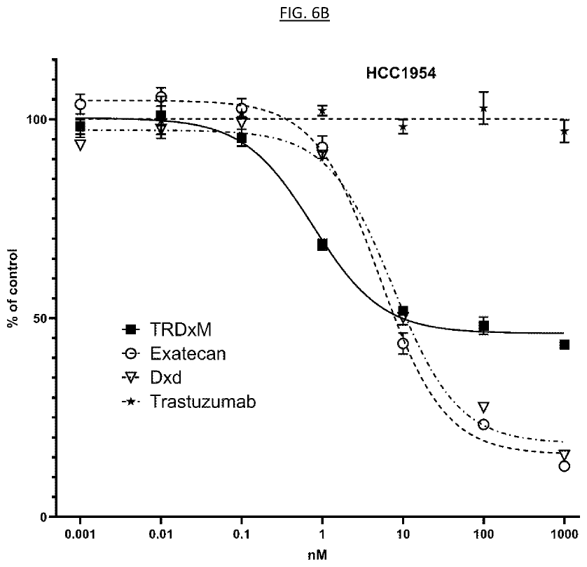

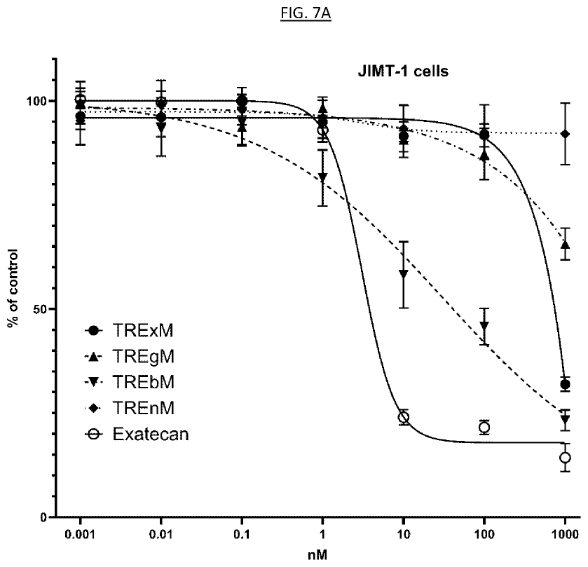

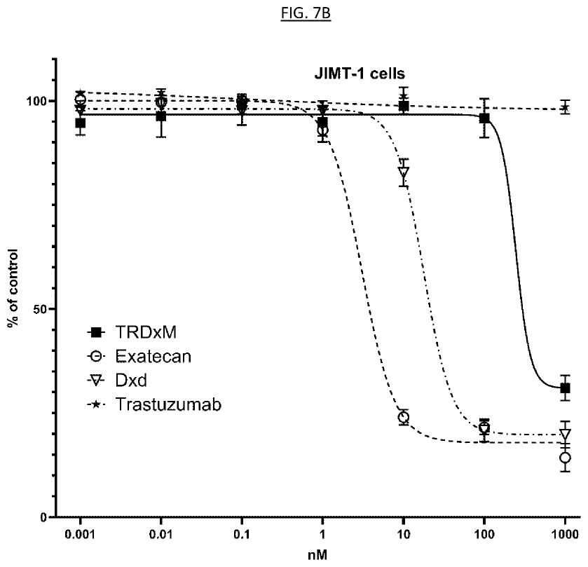

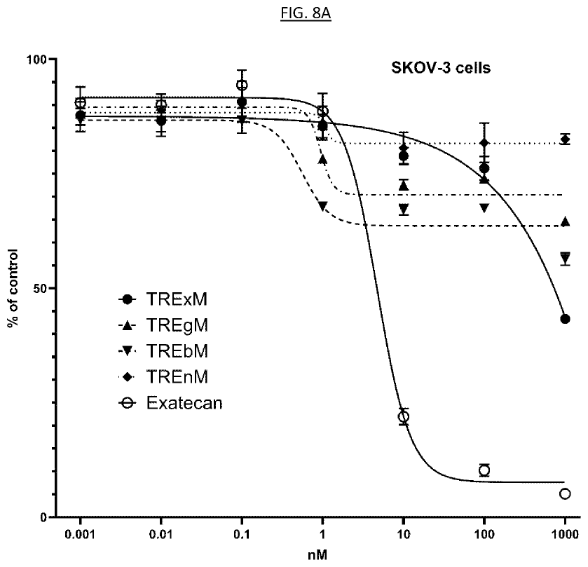

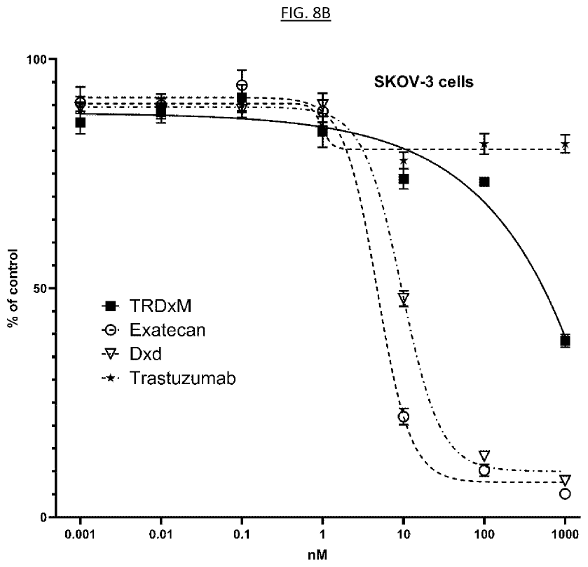

HER2+ SK-BR-3 cells (FIG. 5), HCC1954 cells (FIG. 6), JIMT-1 cells (FIG. 7), and SKOV-3 cells (FIG. 8) were incubated with trastuzumab-DXd ADC TRExM, trastuzumab-exatecan ADC TREgM, trastuzumab-exatecan ADC TREbM, trastuzumab-exatecan ADC TREnM, trastuzumab-deruxtecan ADC TRDxM, free exatecan payload (provided as mesylate salt, free deruxtecan payload (DXd) and trastuzumab.For comparison IC50 values and maximum efficacies (as lowest viability % at), as shown in the legends of FIGS. 5-8 and as discussed below.

TREnM had the lowest cytotoxic activity of the tested ADCs against all the four HER2+ cell lines.

Depending on the cell line, the anti-HER2 cytotoxic activity of TREbM was either comparable with or higher than TREgM. TREbM and TREgM had comparable cytotoxic activity against SK-BR-3, HCC1954 and SKOV-3 cells, while TREbM had higher cytotoxic activity than TREgM against JIMT-1 cells.

Depending on the cell line, the anti-HER2 cytotoxic activities of the exatecan ADCs TREbM and TREgM were either comparable with or higher than the DXd ADCs TRExM and TRDxM. The exatecan ADCs TREbM and TREgM and the DXd ADCs TRExM and TRDxM had comparable cytotoxic activities against HCC1954, while the exatecan ADCs TREbM and TREgM had higher cytotoxic activities than the DXd ADCs TRExM and TRDxM against SK-BR-3 and SKOV-3 cells. TREbM had higher anti-HER2 cytotoxic activity against JIMT-1 cells than either of TREgM, TRExM and TRDxM, which had comparable activity.

Depending on the cell line, the cytotoxic activity free exatecan payload was either comparable with or higher than free DXd payload. Free exatecan and DXd had comparable cytotoxic activity against HCC1954 cells, while free exatecan had higher cytotoxic activity than DXd against SK-BR-3, JIMT-1 and SKOV-3 cells.

Depending on the cell line, trastuzumab either had no activity or very low anti-HER2 cytotoxic activity. Trastuzumab had no cytotoxic activity against HCC1954 and JIMT-1 cells, while trastuzumab had very low cytotoxic activity against SK-BR-3 and SKOV-3 cells.

1. A linker-payload conjugate represented by Formula I or Formula IB 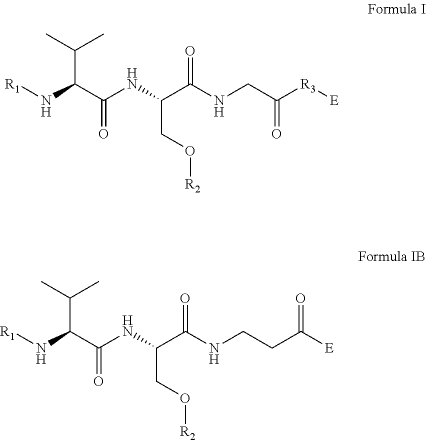
wherein R₁ is a spacer group selected from the group consisting of a maleimidoacetyl, maleimidoacetyl-β-alanyl, and a C₂-C₈ acyl group comprising a bioorthogonal conjugation group; R₂ is selected from the group consisting of a saccharide, a phosphate ester, a sulfate ester, a phosphodiester and a phosphonate; R₃ is a spacer group or absent, wherein the spacer group is selected from the group consisting of a prodrug group and —NH—CH₂—O—CH₂—C(O)—; and E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group.

2. The linker-payload conjugate according to claim 1, wherein the bioorthogonal conjugation group is selected from the group consisting of an azidyl, alkynyl, cycloalkynyl, triazolyl, maleimidyl, thiol, succinimidyl, alkenyl, ether, thioether, —CHO, ketone, hydroxylaminyl, hemiacetal, acetal, phosphinyl, tetrazinyl, cycloalkenyl such as cyclooctenyl, nitronyl, isoxazolinyl, nitrile oxide, tetrazolyl, pyrazolinyl and quadricyclanyl.

3. The linker-payload conjugate according to claim 1, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

4. The linker-payload conjugate according to claim 1, wherein the linked-payload conjugate is represented by any one of Formulas Ia to IBf 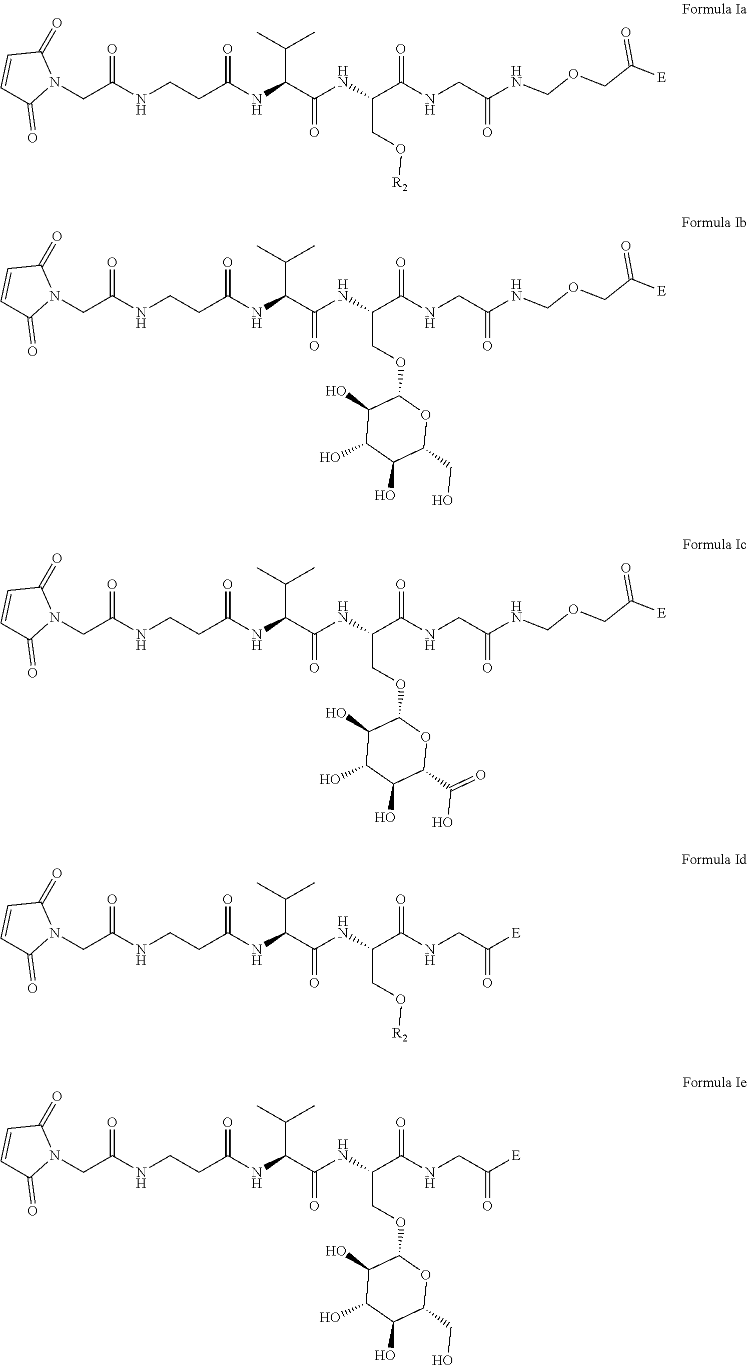 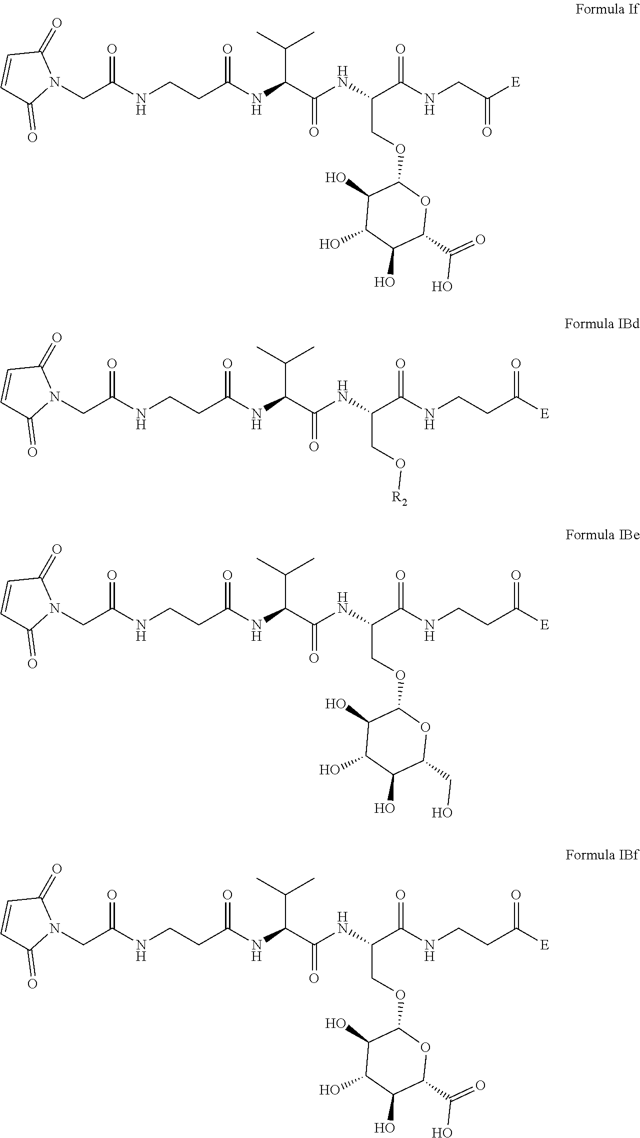
wherein R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification; and E is selected from the group consisting of exatecan and a camptothecin analogue comprising a primary or a secondary amino group, wherein the amino group of E together with the carbonyl group to which E is bonded forms an amide group.

5. The linker-payload conjugate according to claim 1, wherein the linker-payload conjugate is represented by any one of Formulas IIa to IIBf 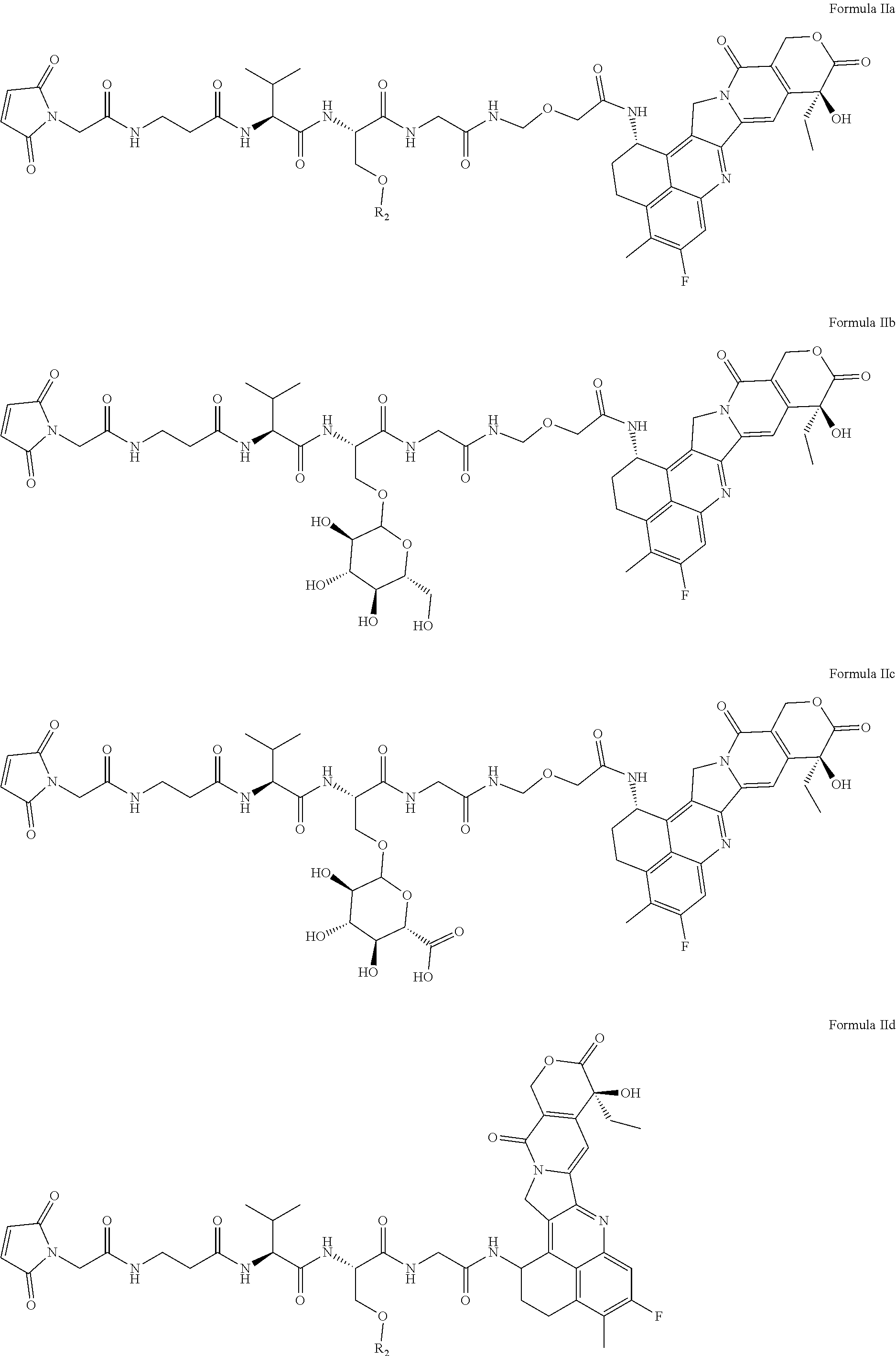 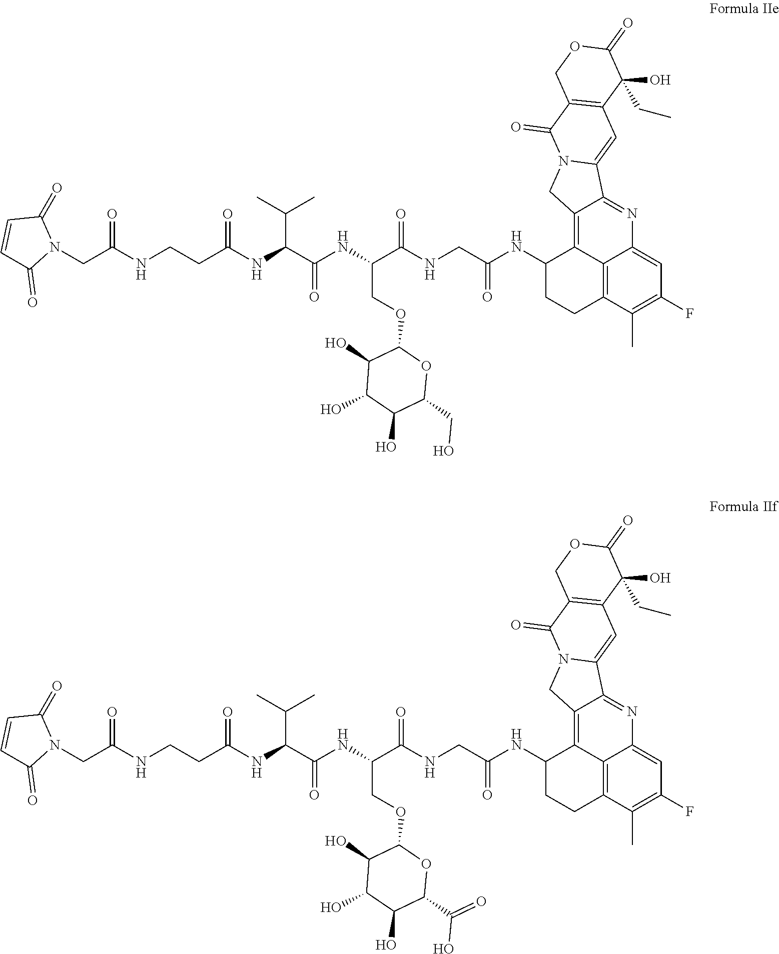 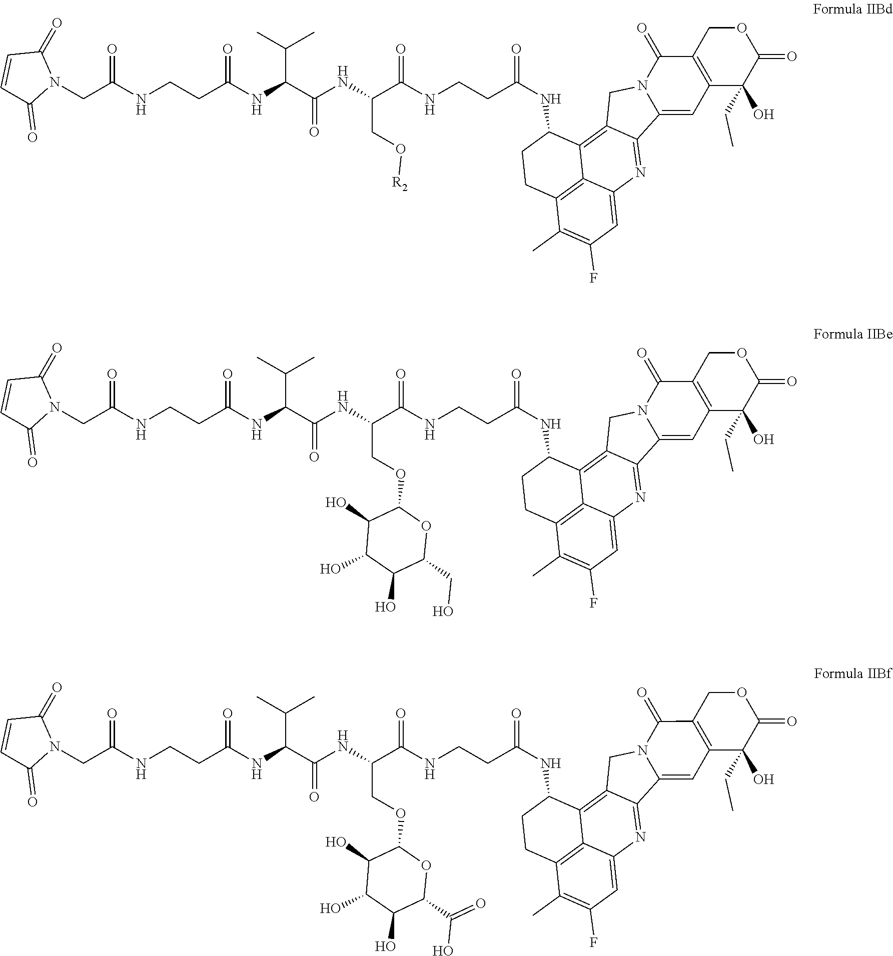
wherein R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

6. The linker-payload conjugate according to claim 1, wherein the linker-payload conjugate is represented by Formula IIb

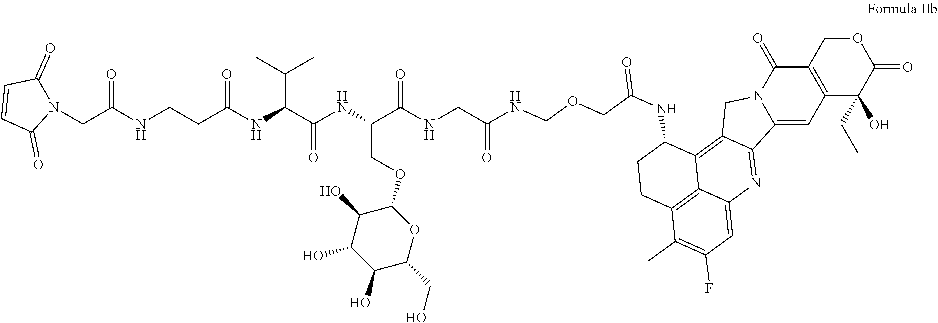 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](CO[C@@H]1O[C@H](CO)[C@@H](O)[C@H](O)[C@H]1O)NC(=O)[C@@H](NC(=O)CCNC(=O)CN1C(=O)C=CC1=O)C(C)C)CC3]

7. The linker-payload conjugate according to claim 1, wherein the linker-payload conjugate is represented by Formula IIe

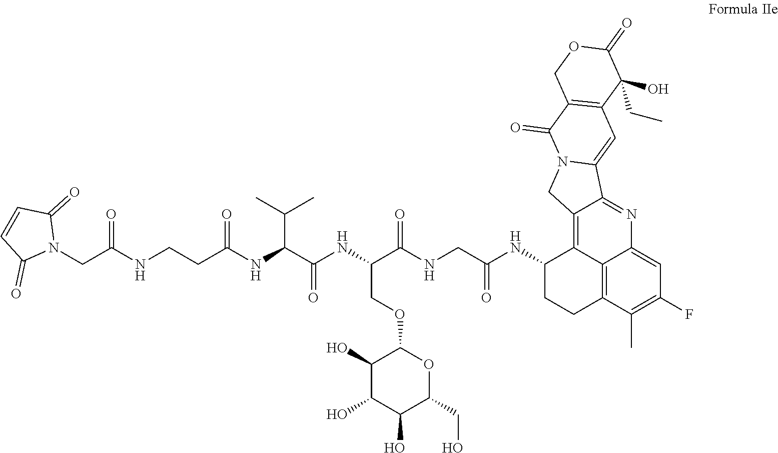 [SMILES: CC[C@@]1(O)C(=O)OCc2c1cc1n(c2=O)Cc2c-1nc1cc(F)c(C)c3c1c2[C@@H](NC(=O)CNC(=O)[C@H](CO[C@@H]1O[C@H](CO)[C@@H](O)[C@H](O)[C@H]1O)NC(=O)[C@@H](NC(=O)CCNC(=O)CN1C(=O)C=CC1=O)C(C)C)CC3]

8. The linker-payload conjugate according to claim 1, wherein the linker-payload conjugate is represented by Formula IIBe

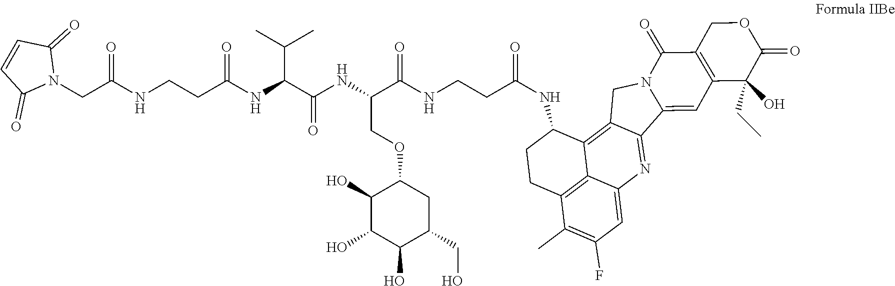 [SMILES: CC[C@@]1(O)C(=O)OCc2c1cc1n(c2=O)Cc2c-1nc1cc(F)c(C)c3c1c2[C@@H](NC(=O)CCNC(=O)[C@H](CO[C@@H]1C[C@H](CO)[C@@H](O)[C@H](O)[C@H]1O)NC(=O)[C@@H](NC(=O)CCNC(=O)CN1C(=O)C=CC1=O)C(C)C)CC3]

9. The linker-payload conjugate according to claim 1, wherein the amine-modified camptothecin analogue is 9-aminocamptothecin.

10. The linker-payload conjugate according to claim 1, wherein the prodrug group is selected from the group consisting of para-aminobenzyloxycarbonyl (PABC), orto-aminobenzyloxycarbonyl, —OCH₂NH—, an amino acid residue, and a peptide.

11. A targeting unit-linker-payload conjugate represented by Formula III or Formula IIIB 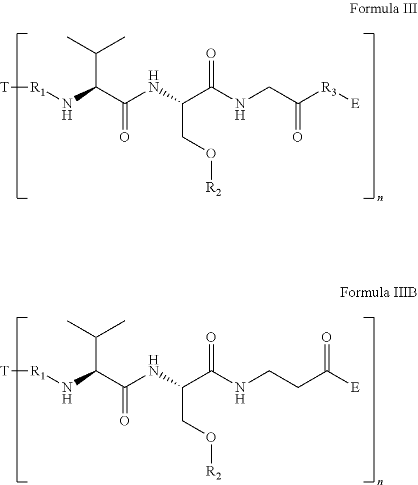
wherein T is a targeting unit; R₁ is a spacer group selected from the group consisting of 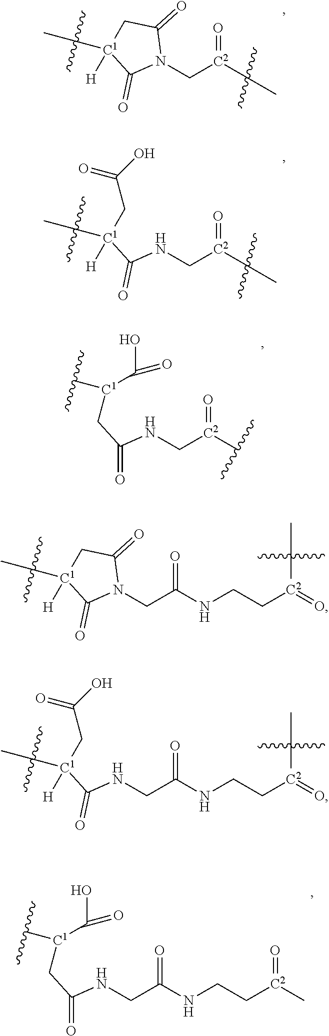
and a C₂-C₈ acyl group comprising a radical of a bioorthogonal conjugation group, wherein C¹ is covalently attached to the targeting unit, optionally covalently attached to to a sulphur of the targeting unit, and C² is covalently attached to the nitrogen of NH; R₂ is selected from the group consisting of a saccharide, a phosphate ester, a sulfate ester, a phosphodiester and a phosphonate; R₃ is a spacer group or absent, wherein the spacer group is selected from the group consisting of a prodrug group and —NH—CH₂—O—CH₂—C(O)—; E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group; and n≥1.

12. The targeting unit-linker-payload conjugate according to claim 11, wherein the radical of a bioorthogonal conjugation group is derived from a group selected from the group consisting of an azidyl, alkynyl, triazolyl, maleimidyl, thiol, succinimidyl, alkenyl, ether, thioether, —CHO, ketone, hydroxylaminyl, hemiacetal, acetal, phosphinyl, tetrazinyl, cyclooctenyl, nitronyl, isoxazolinyl, nitrile oxide, tetrazolyl, pyrazolinyl and quadricyclanyl.

13. The targeting unit-linker-payload conjugate according to claim 11, wherein the radical of the bioorthogonal conjugation group is selected from the group consisting of 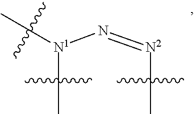
wherein N¹ and N², together with two carbons of the targeting unit to which they are attached to, form a 1,2,3-triazole, and wherein N¹ is also covalently attached to a carbon of the C₂-C₈ acyl group; 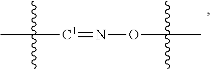
wherein one of C¹ and O is covalently attached to a carbon of the C₂-C₈ acyl group and the other one of C¹ and O is covalently attached to the targeting unit; 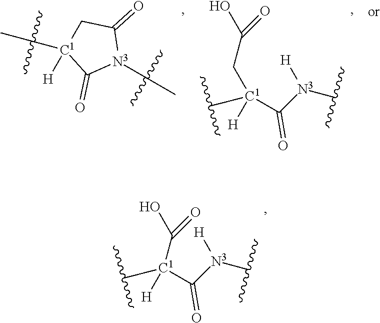
wherein N³ is covalently attached to a carbon of the C₂-C₈ acyl group and C¹ is covalently attached to the targeting unit; 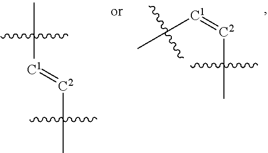
wherein one of C¹ and C² is covalently attached to a carbon of the C₂-C₈ acyl group and the other one of C¹ and C² is covalently attached to the targeting unit; 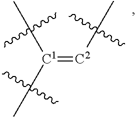
wherein C¹ and C², together with two nitrogens of the targeting unit they are attached to, form a 1,2,3-triazole, and C¹ is also covalently attached to a carbon of the C₂-C₈ acyl group; 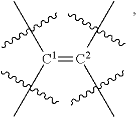
wherein C¹ and C², together with two nitrogens of the targeting unit they are attached to, form a 1,2,3-triazole, and C¹ and C² being part of a cycloalkenyl such as cyclooctenyl or (10Z)-tricyclo [10.4.0.04,9] hexadeca-1(16),4,6,8, 10,12, 14-heptaen-2-yl, which is covalently attached to a carbon of the C₂-C₈ acyl group or to a —O—, —S—, —O₂C—, —O₂CNH—, or —NHCO₂— linker, which is covalently attached to the C₂-C₈ acyl group, or
wherein C¹ and C², together with an oxygen and a carbon of the targeting unit they are attached to, form a N-alkyl isoxazoline, and C¹ and C² being part of a cycloalkenyl such as cyclooctenyl or (10Z)-tricyclo [10.4.0.04,9] hexadeca-1(16),4,6,8,10,12,14-heptaen-2-yl, which is covalently attached to a carbon of the C₂-C₈ acyl group or to a —O—, —S—, —O₂C—, —O₂CNH—, or —NHCO₂— linker, which is covalently attached to the C₂-C₈ acyl group; 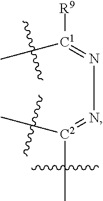
wherein C¹ and C² are covalently attached to two carbons of the targeting unit, and wherein C² is also covalently attached to a carbon of the C₂-C₈ acyl group or to a —O— or —S-linker, which is covalently attached to the C₂-C₈ acyl group, wherein R⁹ is H, C₁₋₅-alkyl, or 2-pyridyl; 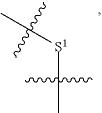
wherein S¹ is covalently attached to the targeting unit and a carbon of the C₂-C₈ acyl group; 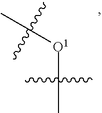
wherein O¹ is covalently attached to the targeting unit and a carbon of the C₂-C₈ acyl group; 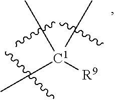
wherein C¹ is covalently attached to a carbon of the C₂-C₈ acyl group and to two oxygens of the targeting unit, and R⁹ is H, or a C₁₋₅-alkyl; 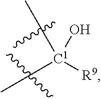
wherein C¹ is covalently attached to a carbon of the C₂-C₈ acyl group and to an oxygen of the targeting unit, and R⁹ is H, or a C₁₋₅-alkyl; 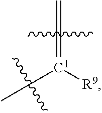
wherein C¹ is covalently attached to a carbon of the C₂-C₈ acyl group and C¹ is also covalently attached, with the double bond, to a nitrogen of the targeting unit, and R⁹ is H, or a C₁₋₅-alkyl; 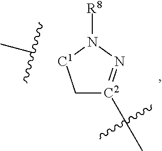
wherein C¹ is covalently attached to a carbon of the C₂-C₈ acyl group or to to a —O—, —CH₂O— or —CH₂OPhCH₂-linker, which is covalently attached to the C₂-C₈ acyl group, and C² is covalently attached to the targeting unit; or wherein C¹ is covalently attached to the targeting unit and C² is covalently attached to a carbon of the C₂-C₈ acyl group or to to a —PhCO₂— linker, which is covalently attached to the C₂-C₈ acyl group, wherein R⁸ is C₁₋₅-alkyl or an optionally substituted phenyl; 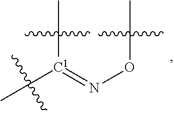
wherein C¹ and O, together with two carbons of the targeting unit they are attached to, form an isoxazoline such as a 2-isoxazoline, 3-isoxazoline, or 4-isoxazoline, and wherein C¹ is also covalently attached to a carbon of the C₂-C₈ acyl group, or wherein C¹ and O, together with two carbons of a cycloalkenyl, such as 2,5-norbornadien-2-yl, form an isoxazoline such as a 2-isoxazoline, 3-isoxazoline, or 4-isoxazoline, wherein the isooxazoline being covalently attached to a carbon of the C₂-C₈ acyl group or to a —O— or —OCH₂— linker, which is covalently attached to the C₂-C₈ acyl group; and 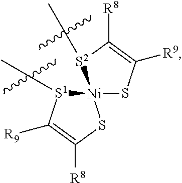
wherein S¹ and S² are covalently attached to the targeting unit, R⁸ is phenyl, and R⁹ is a linker group, such as —PhCONH—, which is covalently attached to the C₂-C₈ acyl group, or one of R⁹ is phenyl and the other one of R⁹ is a linker group, such as —PhCONH—, which is covalently attached to the C₂-C₈ acyl group.

14. The targeting unit-linker-payload conjugate according to claim 11, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

15. The targeting unit-linker-payload conjugate according to claim 11, wherein the targeting unit-linker-payload conjugate is represented by Formula IV or Formula IVB 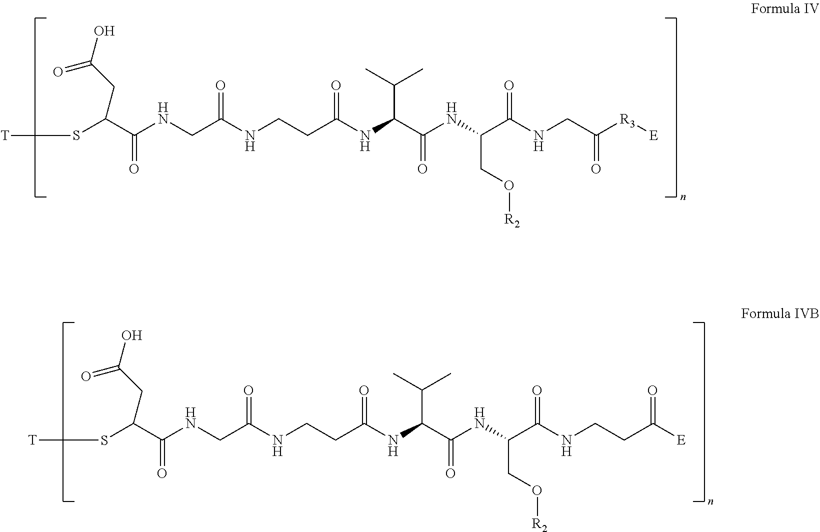
wherein T is an antibody; n is in the range of 1 to about 20; R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification; R₃ is a spacer group or absent, wherein the spacer group is selected from the group consisting of a prodrug group and —NH—CH₂—O—CH₂—C(O)—; E is selected from the group consisting of exatecan and a camptothecin analogue comprising a primary or a secondary amino group, wherein the amino group of E together with a carbonyl group of R₃ or, when R₃ is absent, the amino group of E together with the carbonyl group to which E is bonded forms an amide group.

16. The targeting unit-linker-payload conjugate according to claim 11, wherein the targeting unit-linker-payload conjugate is represented by any one of Formulas IVa to IVBf 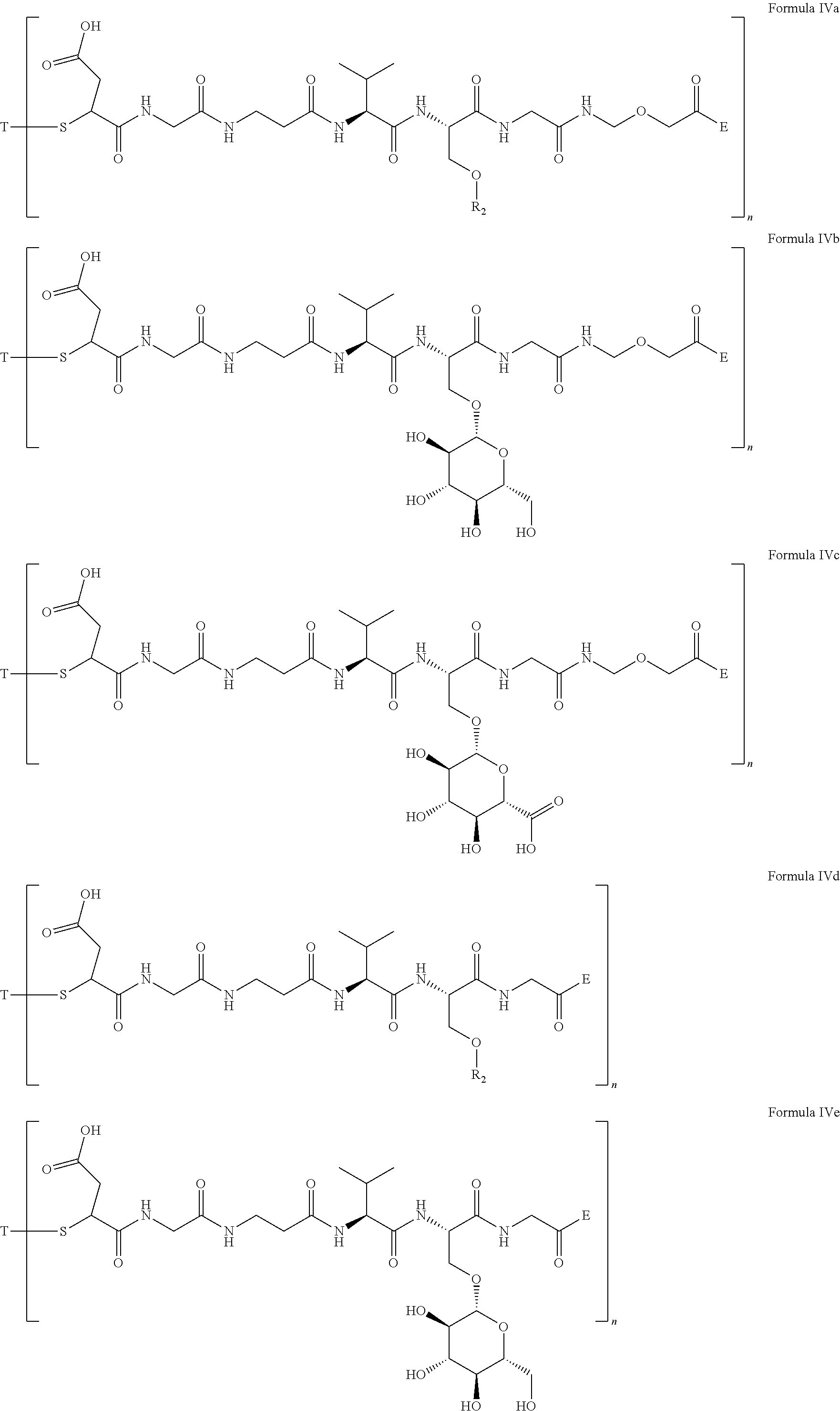 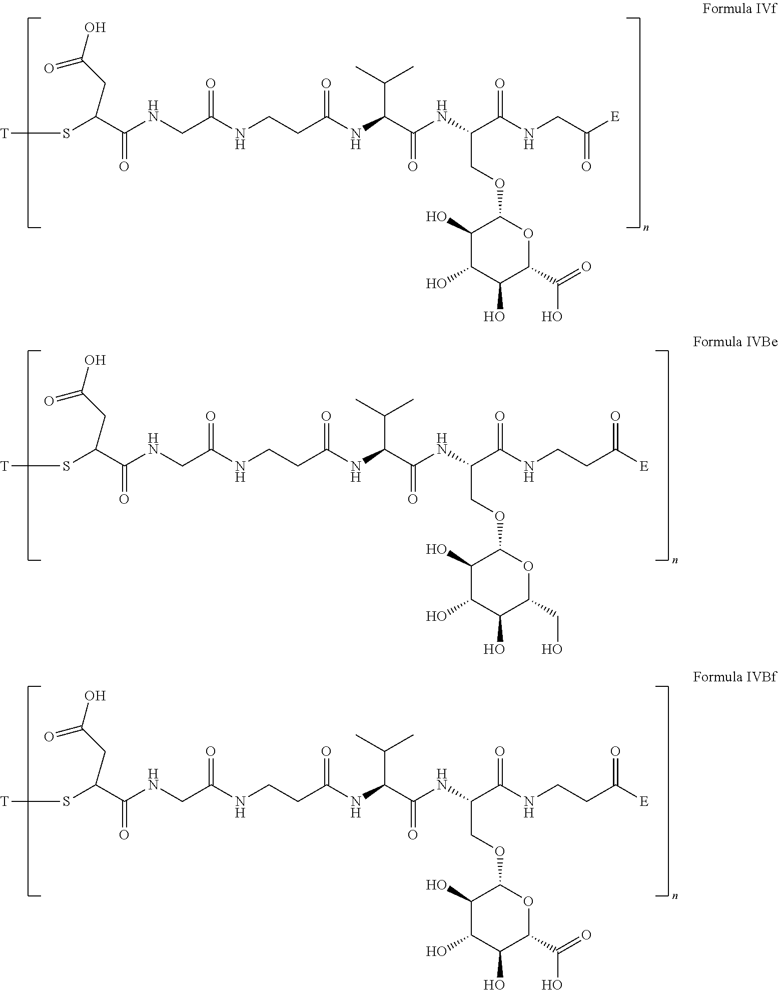
wherein T is an antibody; n is in the range of 1 and about 20; R₂ is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification; E is selected from the group consisting of exatecan and a camptothecin analogue comprising a primary or a secondary amino group, wherein the amino group of E together with the carbonyl group to which E is bonded forms an amide group.

17. The targeting unit-linker-payload conjugate according to claim 11 represented by any one of Formulas Va to VBf 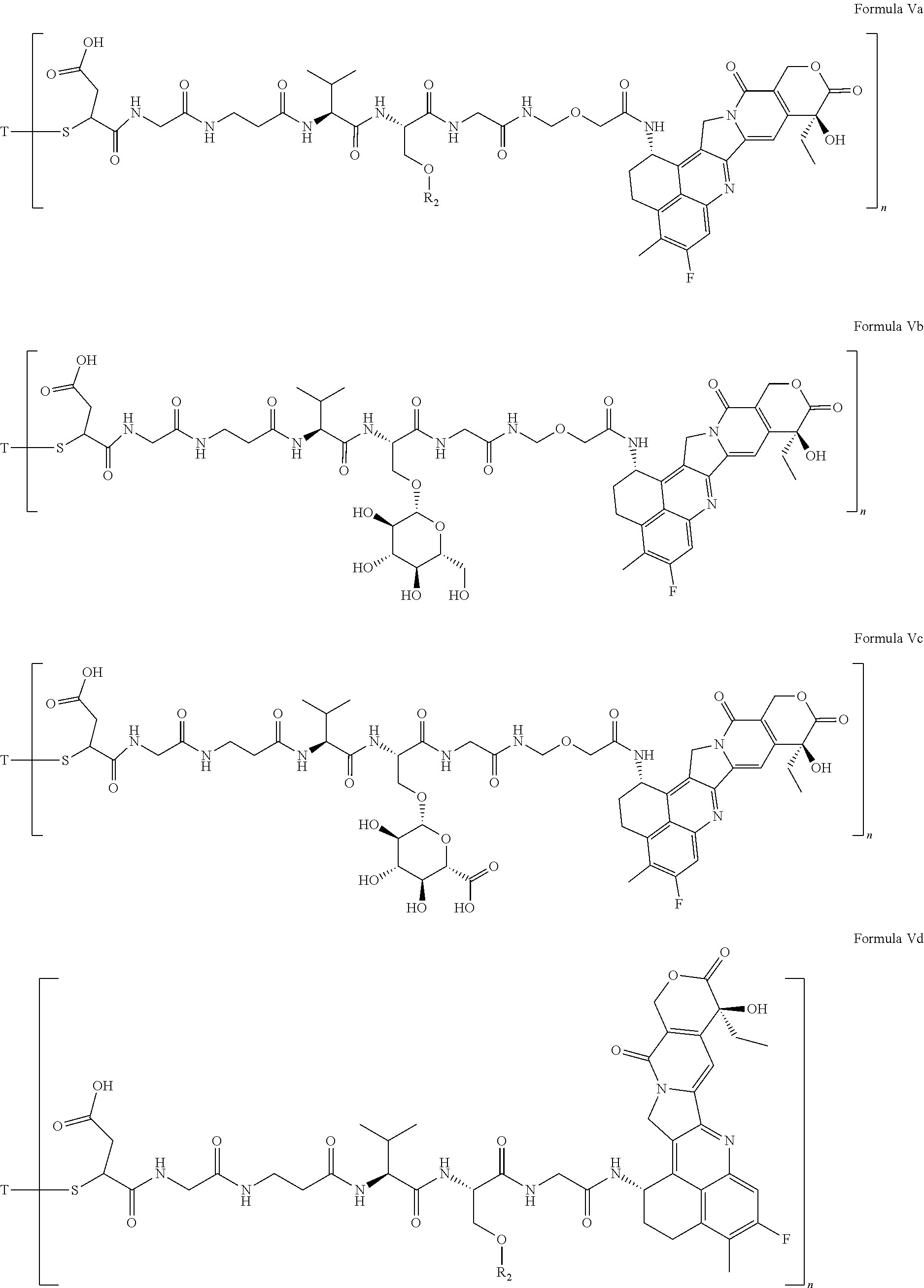 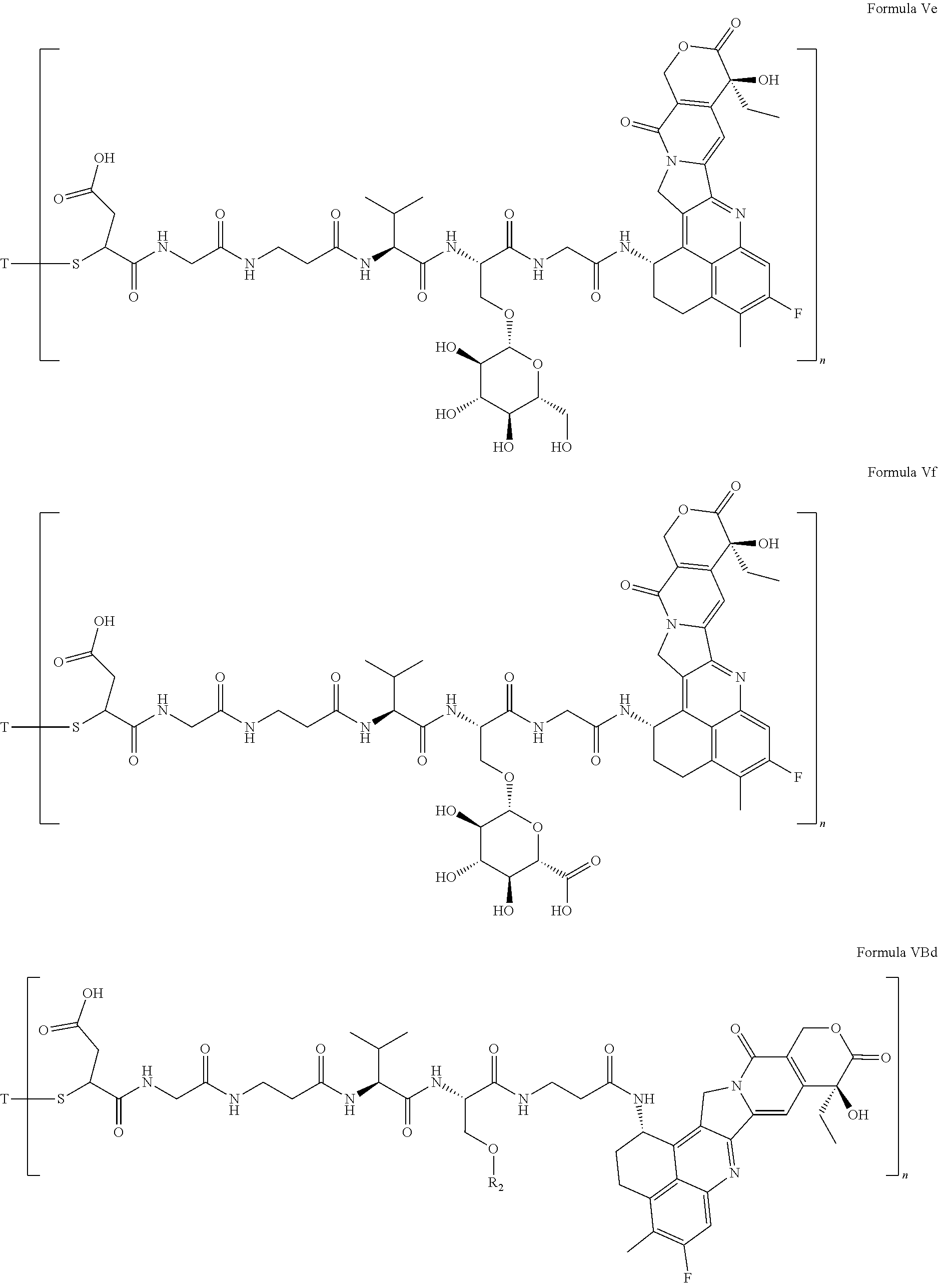 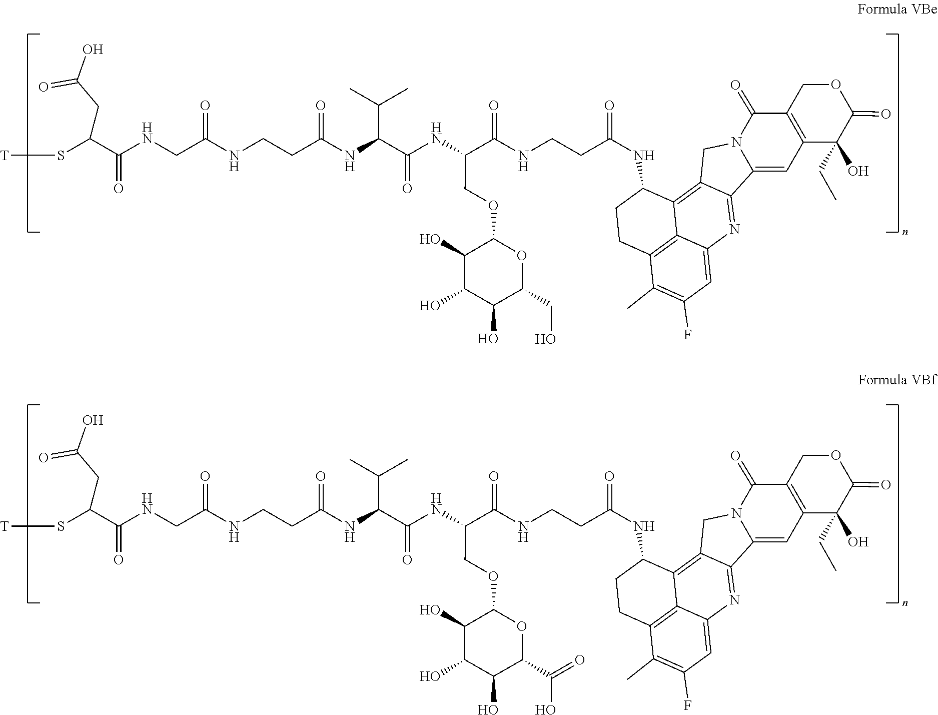
wherein T is an antibody; n is in the range of 1 and about 20; R² is selected from the group consisting of a saccharide, a phosphate ester, a sulfate ester, a phosphodiester and a phosphonate, wherein the saccharide comprises or consists of β-D-galactose, N-acetyl-β-D-galactosamine, N-acetyl-α-D-galactosamine, N-acetyl-β-D-glucosamine, β-D-glucuronic acid, α-L-iduronic acid, α-D-galactose, α-D-glucose, β-D-glucose, α-D-mannose, β-D-mannose, α-L-fucose, β-D-xylose, a neuraminic acid or any analogue or modification thereof; wherein the modification of the saccharide is optionally a sulfate, phosphate, carboxyl, amino, or O-acetyl modification.

18. The targeting unit-linker-payload conjugate according to claim 11, wherein the amine-modified camptothecin analogue is 9-aminocamptothecin.

19. The targeting unit-linker-payload conjugate according to claim 11, wherein the prodrug group is selected from the group consisting of para-aminobenzyloxycarbonyl (PABC), orto-aminobenzyloxycarbonyl, —OCH₂NH—, an amino acid residue, and a peptide.

20. The targeting unit-linker-payload conjugate according to claim 11, wherein the targeting unit is an antibody.

21. The targeting unit-linker-payload conjugate according to claim 20, wherein the antibody is selected from the group consisting of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan, or the antibody is selected from the group consisting of an anti-EGFR1 antibody, an epidermal growth factor receptor 2 (HER2/neu) antibody, an anti-CD22 antibody, an anti-CD30 antibody, an anti-CD33 antibody, an anti-Lewis y antibody and an anti-CD20 antibody.

22. The targeting unit-linker-payload conjugate according to claim 11, wherein n is in the range of 1 to about 20, or in the range of 1 to about 15, or in the range of 1 to about 10, or in the range of 2 to 10, or in the range of 2 to 6, or in the range of 2 to 5, or in the range of 2 to 4; or in the range of 3 to about 20, or in the range of 3 to about 15, or in the range of 3 to about 10, or in the range of 3 to about 9, or in the range of 3 to about 8, or in the range of 3 to about 7, or in the range of 3 to about 6, or in the range of 3 to 5, or in the range of 3 to 4; or in the range of 4 to about 20, or in the range of 4 to about 15, or in the range of 4 to about 10, or in the range of 4 to about 9, or in the range of 4 to about 8, or in the range of 4 to about 7, or in the range of 4 to about 6, or in the range of 4 to 5; or in the range of about 7-9; or about 8; or n is 1, 2, 3, 4, 5, 6, 7, 8, 9, 10, 11, 12, 13, 14, 15, 16, 17, 18, 19, or 20.

23. The targeting unit-linker-payload conjugate according to claim 11, wherein
T is an antibody; n is in the range of 1 to about 20; R₁ is 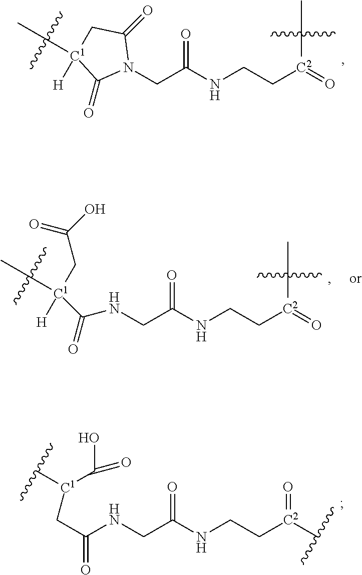
R₂is β-D-glucose; R₃ is —NH—CH₂—O—CH₂—C(O)—; and E is exatecan;
T is an antibody; n is in the range of 1 to about 20; R₁ is 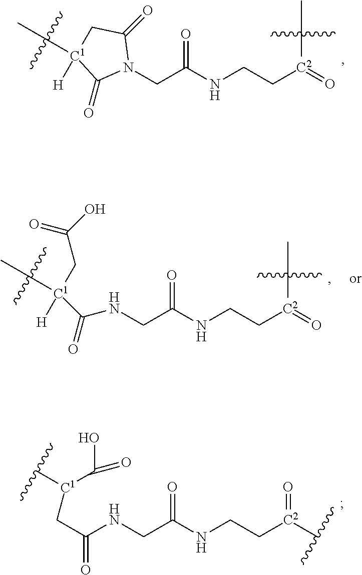
R₂ is β-D-glucuronic acid; R₃ is —NH—CH₂—O—CH₂—C(O)—; and E is exatecan;
T is an antibody; n is in the range of 1 to about 20; R₁ is 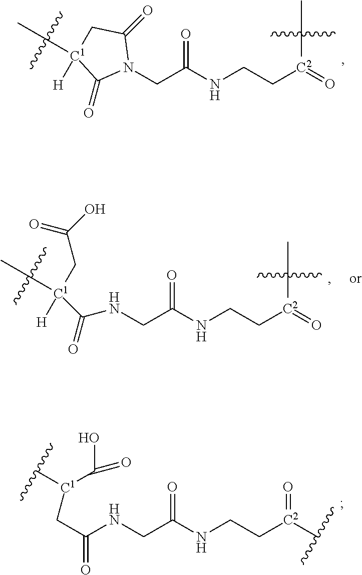
R₂ is β-D-glucose; R₃ is absent; and E is exatecan;
T is an antibody; n is in the range of 1 to about 20; R₁ is 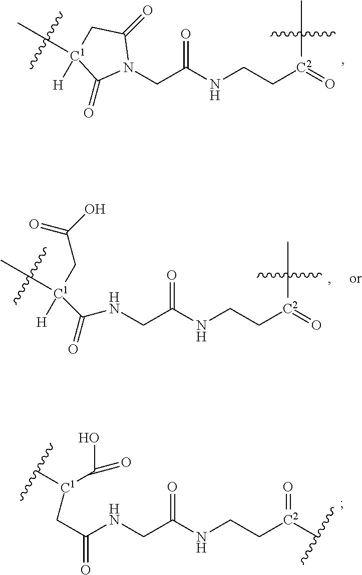
R₂ is β-D-glucuronic acid; R₃ is absent; and E is exatecan;
T is an antibody; n is about 8; R₁ is 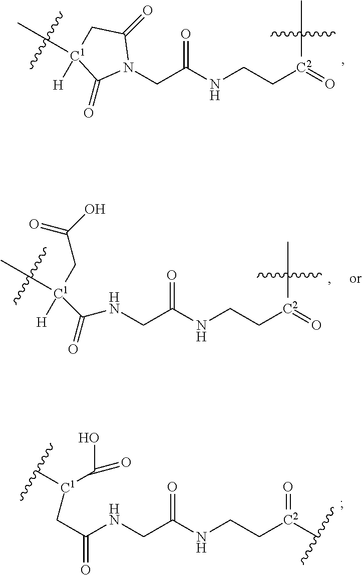
R₂ is β-D-glucose; R₃ is —NH—CH₂—O—CH₂—C(O)—; and E is exatecan;
T is an antibody; n is about 8; R₁ is 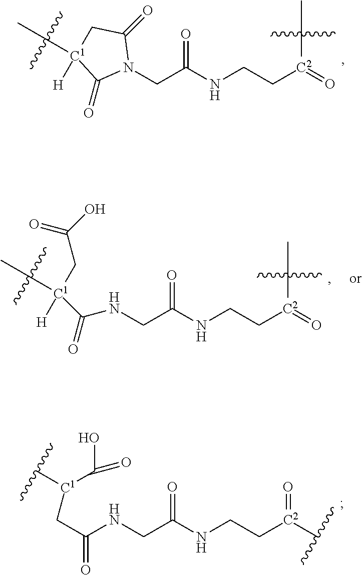
R₂ is β-D-glucuronic acid; R₃ is —NH—CH₂—O—CH₂—C(O)—; and E is exatecan;
T is an antibody; n is about 8; R₁ is 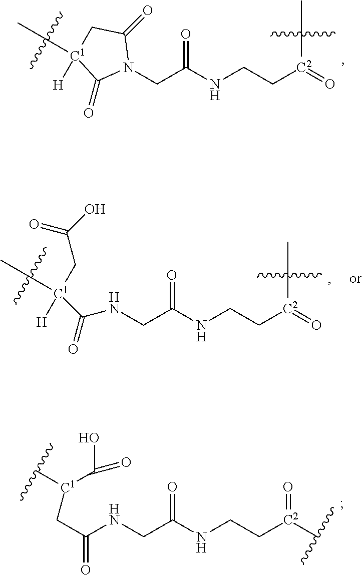
R₂ is β-D-glucose; R₃ is absent; and E is exatecan;
T is an antibody; n is about 8; R₁ is 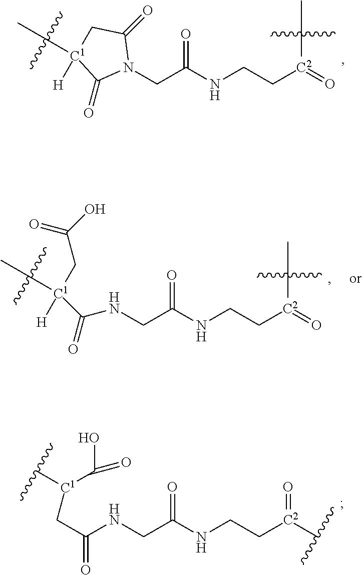
R₂ is β-D-glucuronic acid; R₃ is absent; and E is exatecan;
T is trastuzumab; n is about 8; R₁ is 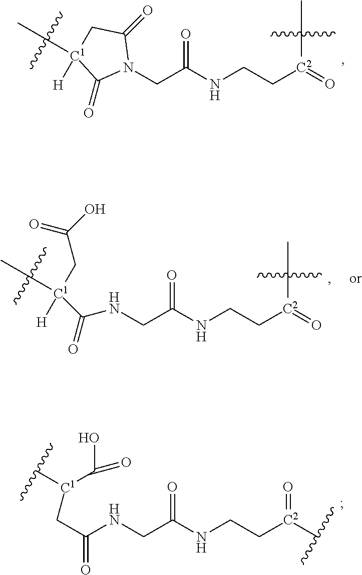
R₂ is β-D-glucose; R₃ is —NH—CH₂—O—CH₂—C(O)—; and E is exatecan;
T is trastuzumab; n is about 8; R₁ is 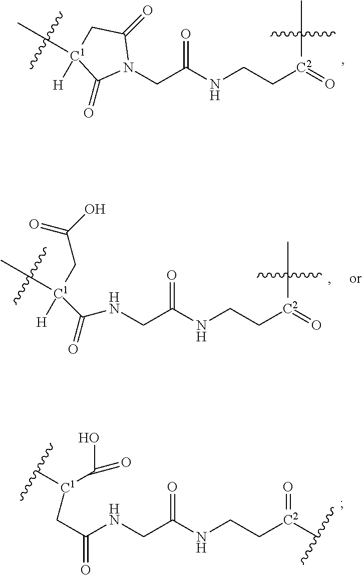
R₂ is β-D-glucuronic acid; R₃ is —NH—CH₂—O—CH₂—C(O)—; and E is exatecan;
T is trastuzumab; n is about 8; R₁ is 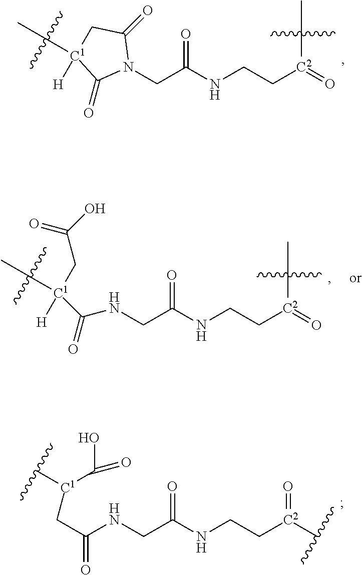
R₂ is β-D-glucose; R₃ is absent; and E is exatecan;
T is trastuzumab; n is about 8; R₁ is 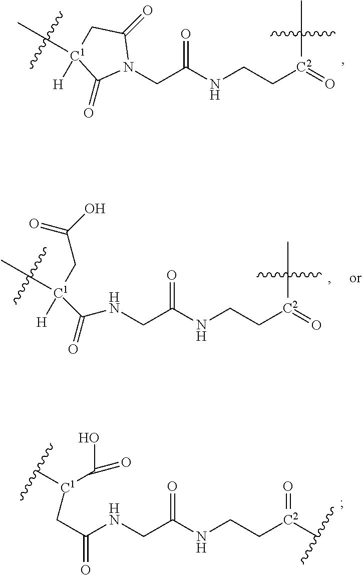
R₂ is β-D-glucuronic acid; R₃ is absent; and E is exatecan;
T is flanvotumab; n is about 8; R₁ is 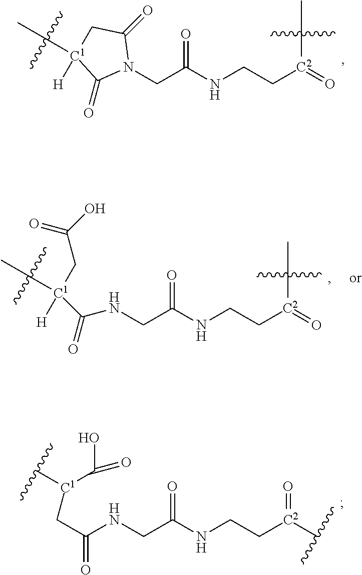
R₂ is β-D-glucose; R₃ is —NH—CH₂—O—CH₂—C(O)—; and E is exatecan;
T is flanvotumab; n is about 8; R₁ is 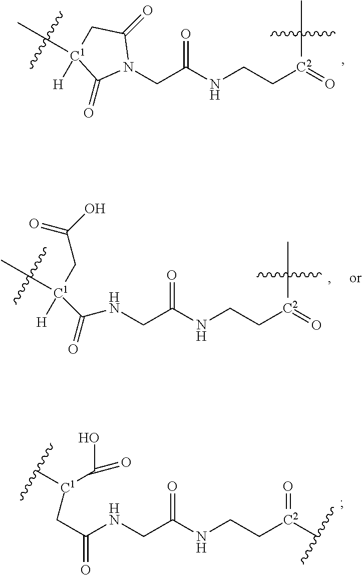
R₂ is β-D-glucuronic acid; R₃ is —NH—CH₂—O—CH₂—C(O)—; and E is exatecan;
T is flanvotumab; n is about 8; R₁ is 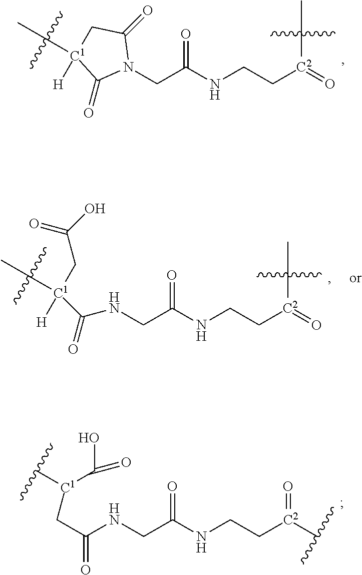
R₂ is β-D-glucose; R₃ is absent; and E is exatecan;
T is flanvotumab; n is about 8; R₁ is 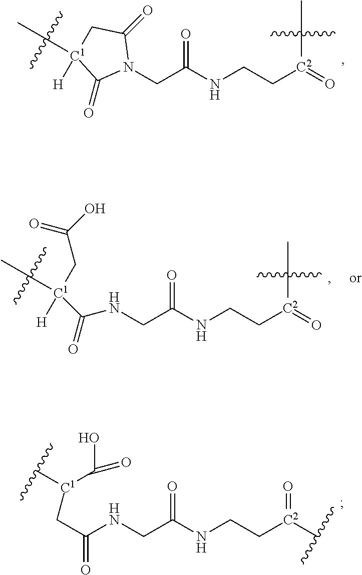
R₂ is β-D-glucuronic acid; R₃ is absent; and E is exatecan;
T is lintuzumab; n is about 8; R₁ is 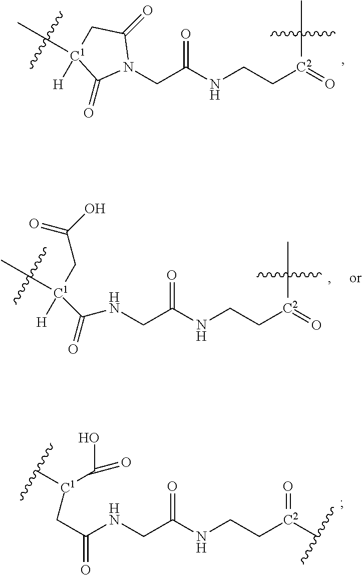
R₂ is β-D-glucose; R₃ is —NH—CH₂—O—CH₂—C(O)—; and E is exatecan;
T is lintuzumab; n is about 8; R₁ is 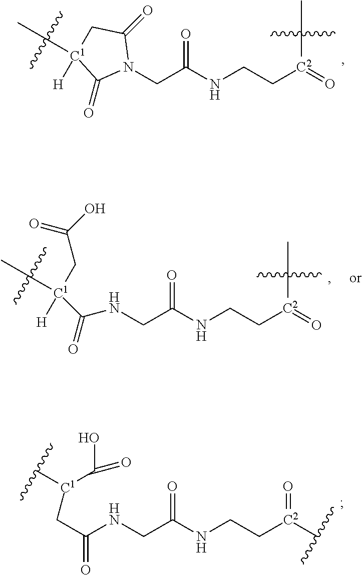
R₂ is β-D-glucuronic acid; R₃ is —NH—CH₂—O—CH₂—C(O)—; and E is exatecan;
T is lintuzumab; n is about 8; R₁ is 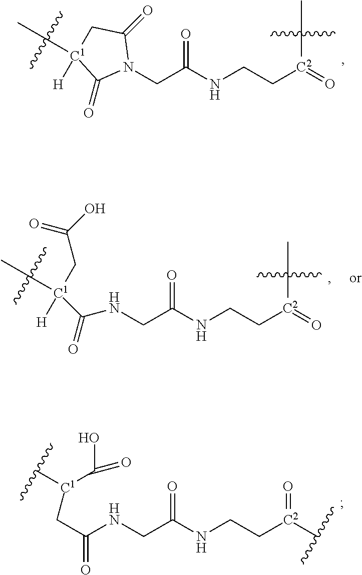
R₂ is β-D-glucose; R₃ is absent; and E is exatecan;
T is lintuzumab; n is about 8; R₁ is 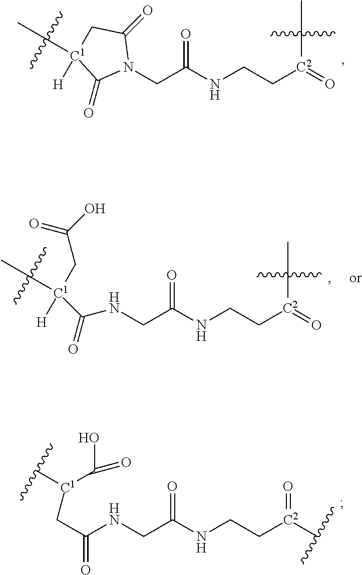
R₂ is β-D-glucuronic acid; R₃ is absent; and E is exatecan;
T is an antibody; n is in the range of 1 to about 20; R₁ is 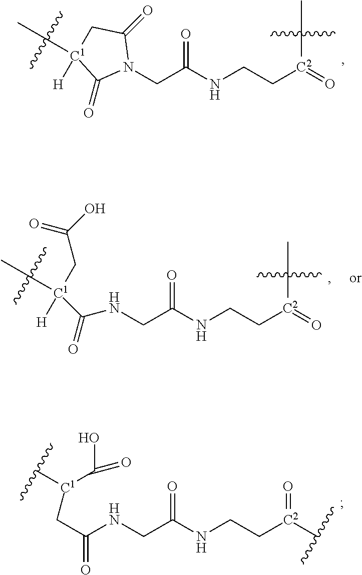
R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is an antibody; n is in the range of 1 to about 20; R₁ is 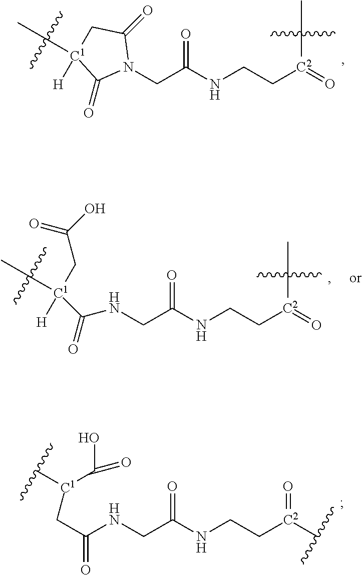
R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is an antibody: n is in the range of 1 to about 20; R₁ is 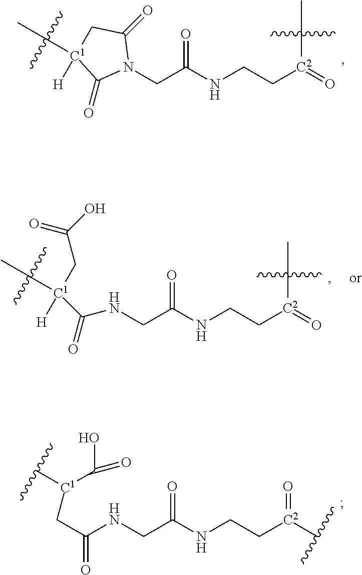
R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is an antibody; n is in the range of 1 to about 20; R₁ is 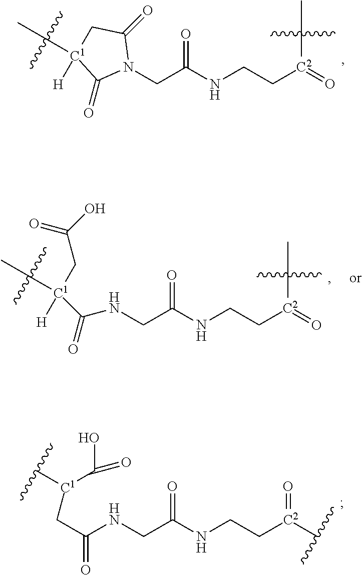
R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is an antibody: n is about 8: R₁ is 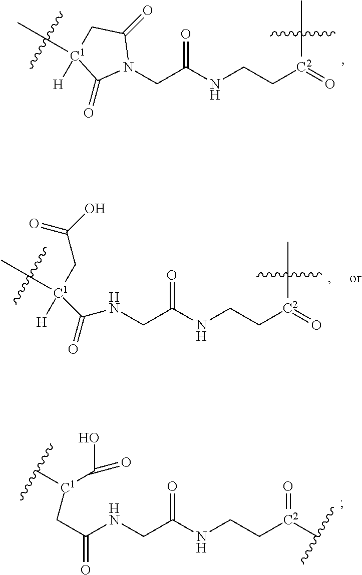
R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is an antibody; n is about 8; R₁ is 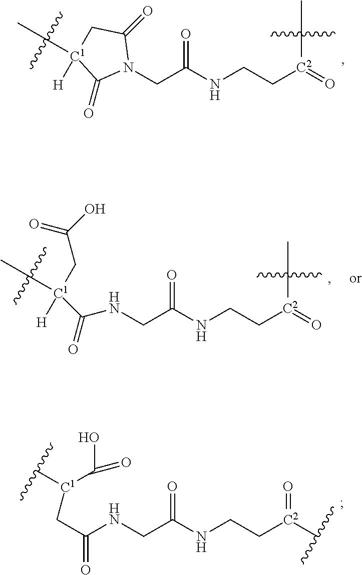
R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is an antibody; n is about 8; R₁ is 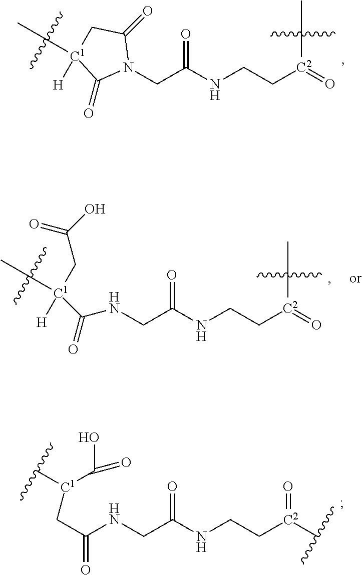
R₂ is β-D-glucose; R₃ is absent; and E is a camptothecin analogue comprising a primary or a secondary amine group, wherein the bond between E and the carbonyl group to which E is bonded is an amide bond to the amino group of the primary or the secondary amino group of the camptothecin analogue;
T is an antibody; n is about 8; R₁ is 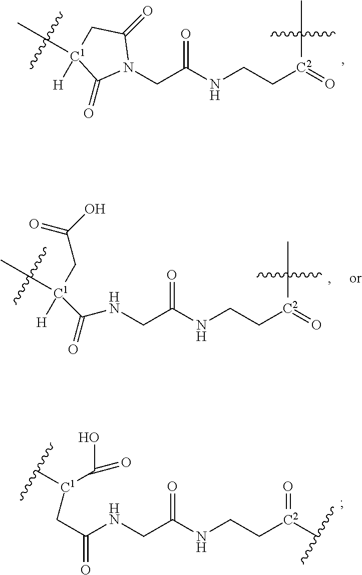
R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is trastuzumab; n is about 8; R₁ is 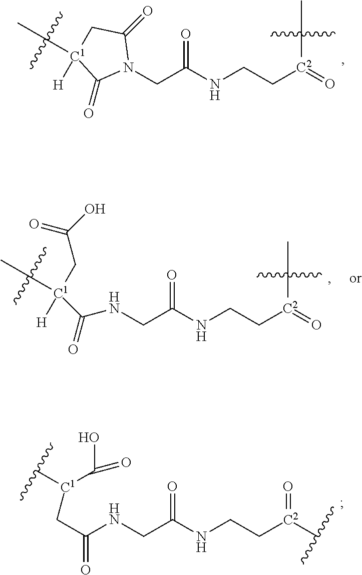
R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is trastuzumab; n is about 8; R₁ is 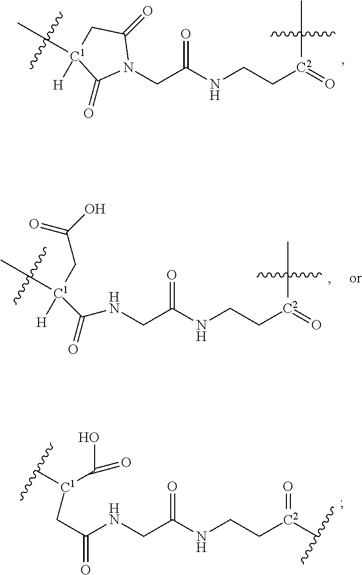
R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is trastuzumab; n is about 8; R₁ is 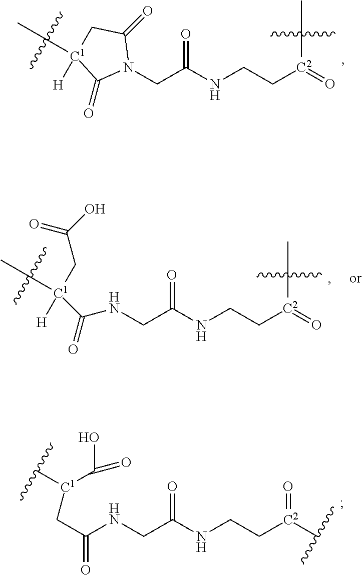
R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group
T is trastuzumab; n is about 8; R₁ is 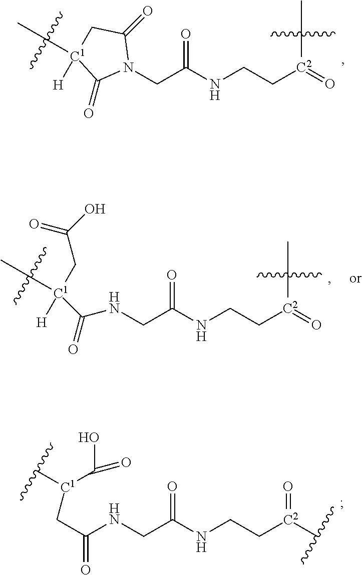
R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is flanvotumab; n is about 8; R₁ is 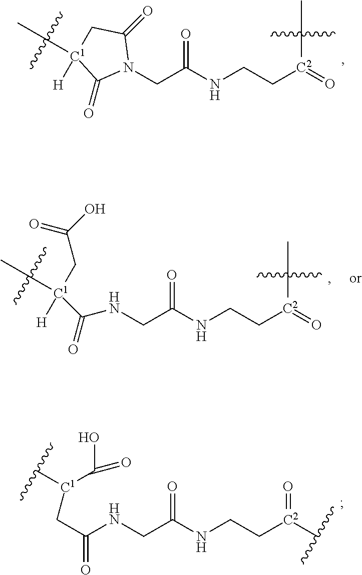
R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is flanvotumab; n is about 8; R₁ is 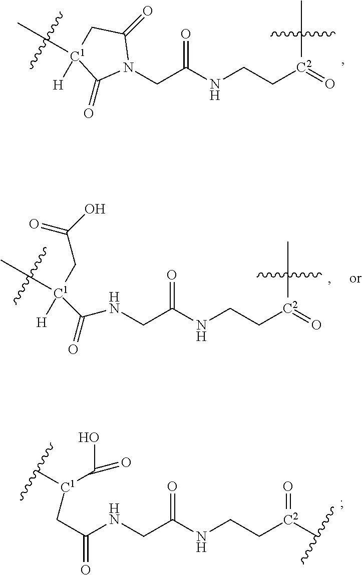
R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is flanvotumab; n is about 8; R₁ is 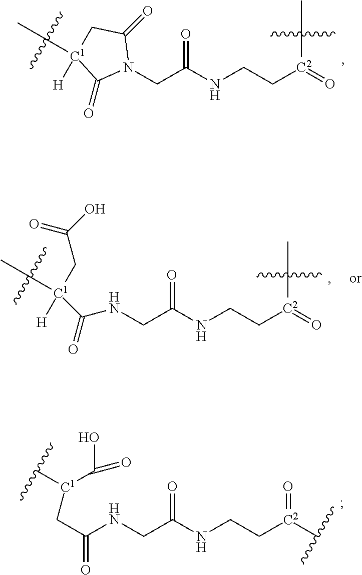
R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is flanvotumab; n is about 8; R₁ is 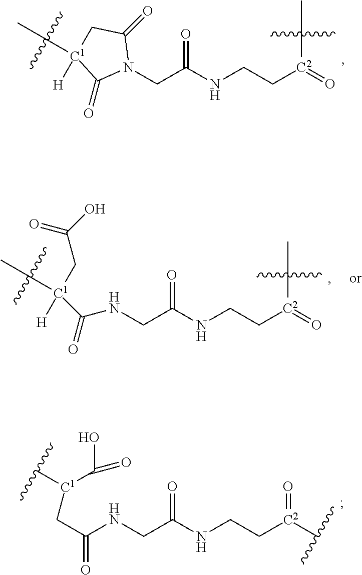
R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is lintuzumab; n is about 8; R₁ is 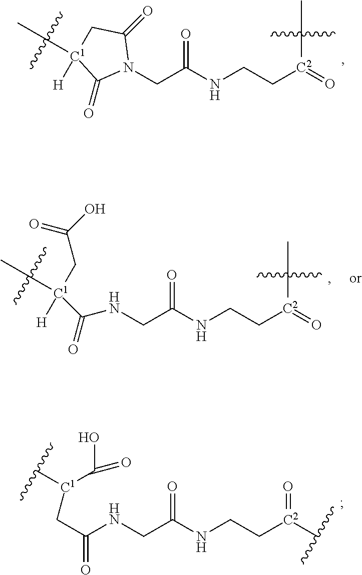
R₂ is β-D-glucose; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is lintuzumab; n is about 8; R₁ is 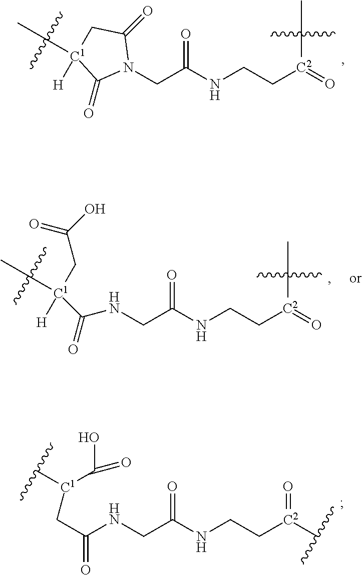
R₂ is β-D-glucuronic acid; R₃ is —NH—CH₂—O—CH₂—C(O)—; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is lintuzumab; n is about 8; R₁ is 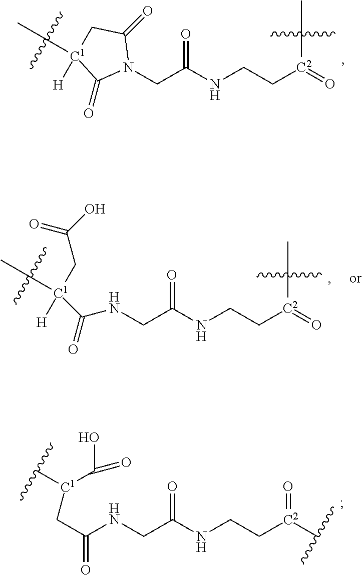
R₂ is β-D-glucose; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group;
T is lintuzumab; n is about 8; R₁ is 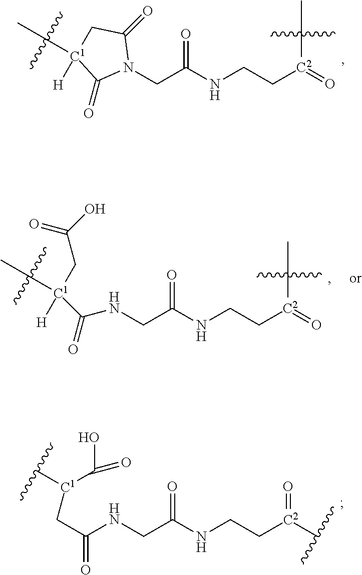
R₂ is β-D-glucuronic acid; R₃ is absent; and E is a camptothecin analogue comprising a primary or a secondary amino group, wherein the primary or secondary amino group of the camptothecin analogue together with the carbonyl group to which the primary or secondary amino group of the camptothecin analogue is bonded forms an amide group.

24. The targeting unit-linker-payload conjugate according to claim 11, wherein the targeting unit-linker-payload conjugate is represented by formula VBe,
wherein T is an antibody, and n is in the range of 1 to about 20; or
T is an antibody, and n is about 8; or
T is trastuzumab, and n is about 8.

25. A pharmaceutical composition comprising the targeting unit-linker-payload conjugate according to claim 11.

26. A method of treating and/or modulating the growth of and/or prophylaxis of tumor cells in a human or an animal, wherein the pharmaceutical composition according to claim 25 is administered to the human or animal in an effective amount.

27. The method according to claim 26, wherein the tumor cells are selected from the group consisting of leukemia cells, lymphoma cells, breast cancer cells, prostate cancer cells, ovarian cancer cells, colorectal cancer cells, gastric cancer cells, squamous cancer cells, small-cell lung cancer cells, head-and-neck cancer cells, multidrug resistant cancer cells, and testicular cancer cells.
